(19) United States
(12) Patent Application Publication
(10) Pub. No.: US 2020/0033351 A1
(43) Pub. Date: Jan. 30, 2020

(54) DIAGNOSTIC METHODS AND KITS FOR EARLY DETECTION OF OVARIAN CANCER

(71) Applicants: Tel Hashomer Medical Research Infrastructure and Service Ltd., Ramat Gan (IL); Ramot at Tel-Aviv University Ltd., Tel-Aviv (IL)

(72) Inventors: Keren LEVANON, Petach Tikva (IL); Tamar Geiger, Tel-Aviv (IL); Keren Bahar-Shany, Tel-Mond (IL); Georgina D. Barnabas, Tel-Aviv (IL); Limor Helpman, Ramat Hasharon (IL); Jacob Korach, Ramat-Gan (IL); Tamar Perri, Mazor (IL)

(21) Appl. No.: 16/588,208

(22) Filed: Sep. 30, 2019

Related U.S. Application Data
(63) Continuation-in-part of application No. PCT/IL2018/050377, filed on Mar. 29, 2018.
(60) Provisional application No. 62/478,778, filed on Mar. 30, 2017.

(51) Int. Cl. G01N 33/574; G01N 33/563; G01N 33/543

(52) U.S. Cl. CPC G01N 33/57449; G01N 33/54306; G01N 33/563

(57) ABSTRACT

The invention relates to novel biomarker signature, diagnostic methods, kits and compositions for early diagnosis of ovarian cancer, based on microvesicles prepared from body fluid sample, specifically, uterine lavage fluid (UtLF) sample.

### FIELD OF THE INVENTION

The invention relates to diagnosis of cancer. More specifically, the present invention provides novel biomarker signature, diagnostic methods, kits and compositions for early diagnosis of ovarian cancer.

### BACKGROUND ART

References considered to be relevant as background to the presently disclosed subject matter are listed below:

[1] Vaughan S, et al., Nat. Rev. Cancer 11: 719-725 (2011)

[2] Havrilesky L J et al., Gynecol Oncol 110:374-382 (2008).

[3] Kozak K R, et al., Proteomics 5:4589-4596 (2005)

[4] Bast Jr. R C, et al., Int J Gynecol Cancer 15 Suppl 3:274-281 (2005)

[5] Moore L E, et al., Cancer 118:91-100 (2012)

[6] Sarojini S, et al., J Oncol 2012:709049 (2012)

[7] Moore R G, et al., Gynecol Oncol 112:40-46 (2009)

[7] Freydanck M K, et al., Anticancer Res 32:2003-8 (2012)

[9] Lu K H, et al., Cancer 119:3454-61 (2013)

[10] Stukan M, et al., J Ultrasound Med 34:207-17 (2015)

[11] Jacobs I J, et al., Lancet 387 :945-956 (2015)

[12] Buys S S, et a., JAMA 305:2295-2303 (2011)

[13] Erickson B K, et al., Obstet Gynecol 124:881-5 (2014)

[14] Kinde I, et al., Sci Transl Med 5:167ra4 (2013)

[15] Maritschnegg E, et al., J Clin Oncol 33:4293-300 (2015)

[16] Krimmel J D, et al., Proc Natl Acad Sci U S A 113:6005-10 (2016)

[17] Harel M, et al., Mol Cell Proteomics 14:1127-1136 (2015)

[18] Levanon K, et al., Oncogene 29:1103-1113 (2009)

[19] Liu X, et al., Int J Oncol 46:2467-7 (2015)

[20] Tucker S L, et al., Clin Cancer Res 20:3280-3288 (2014)

[21] Harmsen M G, et al., BMC Cancer 15:593 (2015)

[22] Arts-de Jong M, et al., Gynecol Oncol 136:305-310 (2015)

[23] George S H, et al., Front Oncol 6:108 (2016)

[24] Levanon K, et al., J Clin Oncol 26:5284-5293 (2008)

[25] Bernardo M M, et al., J Cell Biochem 118(7):1639-47 (2017)

[26] Dean I, et al., Oncotarget 30;8(5):8043-56 (2017)

[27] Maines-Bandiera S, et al., Int J Gynecol Cancer 20(1):16-22 (2010)

[28] Uhlen M, et al., Science 347(6220):1260419-1260419 (2015)

Acknowledgement of the above references herein is not to be inferred as meaning that these are in any way relevant to the patentability of the presently disclosed subject matter.

### BACKGROUND OF THE INVENTION

Overall survival of high grade ovarian cancer (HGOC) patients correlates with disease stage at diagnosis: while patients with stage I disease have >90% 5-year overall survival, rates for stage IV disease are extremely low. Regrettably, HGOC is diagnosed at an advanced stage in ˜75% of the cases regardless of adherence to testing recommendations [1]. This grim reality stems primarily from the lack of effective screening programs and of early stage-specific biomarkers. A multitude of biomarkers have been proposed and tested over the years but none have shown to be effective in improving survival [2-5]. The FDA-approved serum-based biomarkers are CA125 (50-62% sensitivity and 94-98% specificity) and human epididymis protein (HE4) (73% sensitivity at 95% specificity) [6], either individually or in combination [7-8] or their combination with clinical and imaging parameters [9-10]. The recently published UKCTOCS study showed a modest effect on survival with implementation of a specific blood CA125-based monitoring algorithm, which was significant only during years 7-14 of the follow-up [11]. Additionally, the large-scale prostate, lung, colon and ovarian cancer (PLCO) screening study failed to show reduced ovarian cancer-related mortality in 39,105 intermediate risk women who were screened using semiannual plasma CA125 levels and transvaginal ultrasound [12]. Low predictive value stems from the correlation of blood-borne proteins with tumor volume, and hence failure to diagnose the earliest, potentially curable lesions before they have disseminated beyond the ovary. Despite these limitations, plasma-based biomarkers remain the mainstay of all screening approaches, due to the high compliance and accessibility.

Early-detection of HGOC among high-risk population, such as germline BRCA1/2 mutation carriers, is of exceptional importance, as these women are currently counselled to undergo risk reducing bilateral salpingo-oophorectomy (RRBSO) at age ˜40. The dramatic benefit from RRBSO often contrasts with reproductive plans and morbidity of early menopause, thus appealing for a personalized risk assignment method [21, 22]. As a result of these considerations, a novel approach was sought towards development of biomarker for early-detection of HGOC among high-risk populations.

The most common histological subtype of HGOC, high grade serous papillary carcinoma, arises from precursor lesions that develop in the epithelium of the fallopian tube fimbriae (FTE) rather than from the ovarian surface epithelium [23, 24]. It is, therefore, conceivable that detection of early premalignant lesions can be made possible by approaching and sampling the cells of the fimbriae and their secreted biological products (i.e. proteins, cell-free RNA and DNA) through the lower reproductive tract. In contrast to serum biomarkers, locally secreted substances may be detectable at an early-enough stage, thus potentially leading to improved survival. Recently, several groups introduced new methods for the collection of “liquid biopsies” of HGOC tumor in proximity to its origin. Three proof-of-principle studies showed that somatically mutated TP53 from HGOC cells can be isolated from Papanicolaou cytology smears, from vaginal tampons and from uterine washings [13-15], with sensitivity of 41%, 60%, and 60%, respectively. Given that these studies recruited mostly advanced-stage HGOC patients, these sensitivity rates are considered too low. In another study, ultra-deep duplex sequencing detected low frequency mutant TP53 alleles in cells from peritoneal lavage of 94% (16/17 cases) of HGOC patients, but also in 95% of healthy controls (19/20 cases), with a similar frequency and characteristics [16]. This high resolution sequencing technique was also applied to circulating DNA in the blood of patients and controls and detected at least one low frequency TP53 mutation in all cases (15/15) precluding the utility of this method for early detection [16]. There is therefore need for reliable, sensitive and rapid diagnostic methods for early diagnosis of ovarian cancer.

Proteomics is one of the most potent methods in biomedical research, which enables large-scale characterization of proteins in a biological system. Identification of serum/plasma protein biomarkers remains the ‘Holy Grail’ of proteomics. However, deep proteomic profiling of any body-fluid is challenged by the vast dynamic range of their proteomes and the masking of low abundance biomarkers by core plasma proteins, such as albumin, IgG, hemoglobin etc. Recently, the inventors developed a method that overcomes this hurdle, based on isolation of microvesicles from body fluids, followed by high resolution mass spectrometric (MS) analysis [17]. Microvesicles (100 nm-1 μm) are derived from the outward budding of plasma membrane, and they are released into body fluids from all cell types. They consist of proteins, lipids and nucleic acids and their functions depend on the cells of origin. Thus microvesicles can serve as a reservoir of predictive biomarkers, which forms the basis for diagnostic test development, monitoring disease recurrence and treatment response.

The above need of reliable, sensitive and rapid diagnostic methods for early diagnosis of ovarian cancer is addressed by the methods and kits of the invention that provide diagnostic screening test performed on a body fluid sample obtained from the gynecologic tract by a minimally-invasive procedure.

### SUMMARY OF THE INVENTION

In a first aspect, the invention provides a diagnostic method for detecting ovarian cancer in a subject. More specifically, the method of the invention may comprise the steps of: In a first step (a) determining the expression level of at least one biomarker protein in at least one biological sample of said subject, to obtain an expression value for each of said at least one biomarker protein/s. More specifically, the proteins may be selected from Calcium-activated chloride channel regulator 4 (CLCA4), Oviduct-specific glycoprotein (OVGP1), S100 calcium binding protein A14 (S100A14), Small proline-rich protein 3 (SPRR3), Eosinophil cationic protein (RNASE3), Serpin Family B Member 5 (SERPINB5), Clusterin-associated protein 1 (CLUAP1), Carcinoembryonic antigen-related cell adhesion molecule 5 (CEACAM5) and Ectonucleotide pyrophosphatase/phosphodiesterase family member 3 (ENPP3), or any combination thereof. In the next step (b), the method of the invention involves determining if the expression value obtained in step (a) for each of the at least one biomarker protein/s is positive or negative with respect to a predetermined standard expression value or alternatively or additionally, to the expression value of said biomarker protein/s in at least one control sample. In some specific embodiments, a result of at least one of (i) a positive expression value of at least one of the SPRR3, SERPINB5, CEACAM5, S100A14, CLCA4 and biomarker protein/s in the tested sample, indicates that the subject belongs to a predetermined population suffering from ovarian cancer; and (ii) a negative expression value of at least one of the OVGP1, CLUAP1, ENPP3 and RNASE3 biomarker protein/s in said sample, indicates that the subject may be diagnosed as a subject that develops or suffers from an ovarian cancer.

In yet a further aspect, the invention relates to a diagnostic composition comprising at least one detecting molecule or any combination or mixture of plurality of detecting molecules specific for determining the level of expression of at least one of CLCA4, OVGP1, S100A14, SPRR3, RNASE3, SERPINB5, CLUAP1, CEACAM5 and ENPP3 protein/s or any combination thereof, wherein each of said detecting molecules may be specific for one of said biomarker protein/s. In yet a further aspect, the invention relates to a kit comprising: (a) at least one detecting molecule specific for determining the level of expression of at least one of CLCA4, OVGP1, S100A14, SPRR3, RNASE3, SERPINB5, CLUAP1, CEACAM5 and ENPP3 protein/s or any combination thereof in a biological sample. It should be noted that each of the detecting molecule/s may be specific for one of said biomarker proteins. It should be noted that the kit optionally further comprises at least one of: (b) pre-determined calibration curve/s or predetermined standard/s providing standard expression values of said at least one biomarker/s; and (c) at least one control sample.

These and further aspects of the invention will become apparent by the hand of the following drawing.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1E. UtL microvesicle proteomics

FIG. 1A. Workflow from Uterine lavage (UtL) sample collection, microvesicle isolation, peptide purification to Mass spectrometry (MS) analysis.

FIG. 1B. Dynamic range of selected proteins in UtL samples ranging from high abundant known ovarian markers to low abundant cytokines and growth factors.

FIG. 1C. Microvesicle proteomics of the discovery set. Bar plot showing the number of proteins identified in each UtL sample included in the discovery cohort.

FIG. 1D. Label-free quantification (LFQ) intensities of known markers CA125 (MUC16) and HE4 (WFDC2) in log2 normalized intensities.

FIG. 1E. Lack of batch effect of UtL samples. Correlations between protein levels, between patients and controls, and between medical centers.

FIG. 2A-2D. Development of a proteomic classifier for diagnosis of HGOC

FIG. 2A. Comparison of sensitivity, specificity and AUC for the top ranked 5, 9, 14 and 19 overlapping features from different feature ranking methods.

FIG. 2B. Venn diagram showing the selected 9 overlapping features in the top 15 ranks from three different methods.

FIG. 2C. Heatmap showing the expression of 9-protein signature across the discovery set of UtL samples.

FIG. 2D. Confusion matrix and ROC curve show the performance of the 9-protein classifier.

FIG. 3. Expression of the proteomic signature in the UtL discovery set

The individual and average expression as measured by MS is plotted for each of the protein across the HGOC patients and control cohorts. * for p-value<0.05, ** for p-value<0.01.

FIG. 4A-4C. Performance of the proteomic signature on a validation set

FIG. 4A. Schematic workflow of biomarker signature discovery and prediction of the validation set of samples.

FIG. 4B. Confusion matrix.

FIG. 4C. ROC curve of the independent validation cohort, with AUC=0.72.

FIG. 5. Protein expression patterns of 9-protein signature in the early stage HGOC samples

The MS expression of each of the 9-protein signature is more similar to the averaged patients cohort than the averaged control cohort. * for p-value<0.05, ** for p-value<0.01.

FIG. 6. Principal component analysis (PCA) plot of proteomic profile of HGOC patients UtL samples of patients that were previously treated with NACT are not distinguished from those of untreated patients.

FIG. 7A-7B. Characteristics of the NACT-treated HGOC patients' samples in the validation set

FIG. 7A. Clinico-pathological response quality, disease stage and the classifier prediction results for all HGOC patients' samples in the validation set are plotted. Samples collected from patients who had complete or moderate response are highlighted within black box. Abbreviations: TP (true positive), FN (false negative).

FIG. 7B. ROC curve of the validation set after exclusion of the 8 UtL from patients who had moderate/complete response to NACT.

FIG. 8A-8I. Expression of the signature proteins in normal FTE and HGOC

The mRNA levels of the 9-protein signature, specifically, OVGP1 (FIG. 8A), ENPP3 (FIG. 8B), CLUAP1 (FIG. 8C), S100A14 (FIG. 8D), SERPINB5 (FIG. 8E), SPRR3 (FIG. 8F), CEACAM5 (FIG. 8G), RNASE3 (FIG. 8H), CLCA4 (FIG. 8I), from fresh independent normal FTE (n=10) and unmatched HGOC (n=10) specimens, were measured using RT-PCR. Statistical significance of DE marked by * for p-value<0.05 and ** for p-value<0.005.

FIG. 9A-9C. Intensity of IHC staining of SERPINB5, S100A14 and OVGP1 in HGOC tumors and normal FTE

FIG. 9A. shows Tissue Microarrays (TMAs) of HGOC tumors (n=45), and cores of normal fimbriae from patients who underwent salpingectomy due to HGOC, tubal ectopic pregnancy (EP), leiomyomatous uterus (LM), or RRBSO (n=60 each), immunostained for SERPINB5scored on a 0-3 intensity scale and analyzed.

FIG. 9B. shows TMAs of HGOC tumors (n=45), and cores of normal fimbriae from patients operated for HGOC, EP, LM, or RRBSO (n=60 each), immunostained for S100A14 scored on a 0-3 intensity scale and analyzed.

FIG. 9C. shows TMAs of HGOC tumors (n=45), and cores of normal fimbriae from patients operated for HGOC, EP, LM, or RRBSO (n=60 each), immunostained for OVGP1 scored on a 0-3 intensity scale and analyzed.

Score scale was as follows: 0—no staining or faint staining in <10% of cells, 1—faint staining in >10% of cells, 2—intermediate staining of >10% of cells, or strong staining of 10-50% of cells, and 3—strong staining of >50% of cells.

FIG. 10A-10B. Expression of SERPINB5 in HGOC tumors and benign FTE

FIG. 10A. Representative HGOC tumor sections depicting SERPINB5 staining intensities (in brown, 0-3, left to right).

FIG. 10B. Representative sections of fimbriae from the control TMAs (HGOC, EP, LM, RRBSO, left to right) showing minimal or negative immunoreactivity. Scale bar=50 μm, ×400 magnification.

FIG. 11A-11B. Expression of S100A14 in HGOC tumors and benign FTE

FIG. 11A. Representative HGOC tumor sections depicting S100A14 staining intensities (0-3, left to right).

FIG. 11B. Representative sections of fimbriae from the control TMAs (HGOC, EP, LM, RRBSO, left to right). Ciliated cells stain strongly positive while staining of secretory FTE is generally low. Scale bar=50 μm.

FIG. 12A-12B. Expression of OVGP1 in HGOC tumors and benign FTE

FIG. 12A. Representative normal FTE sections of HGOC patients depicting OVGP1 staining intensities (in brown, 0-3, left to right).

FIG. 12B. Representative sections of HGOC tumor and fimbriae from the control TMAs (EP, LM, RRBSO, left to right). Normal fimbriae demonstrate strong confluent membranous staining. Scale bar=50 μm, X400 magnification.

FIG. 13A-13B. Expression of the 9-protein signature across the BRCA mutation carriers cohort

FIG. 13A. Heatmap representing the relative expression of each protein in each sample of BRCA carrier cohort, including HGOC patients, controls and healthy women at high-risk.

FIG. 13B. Averaged expression of the 9-protein signature in the 3 sub-groups of BRCA carriers. * for p-value<0.05, ** for p-value<0.01.

### DETAILED DESCRIPTION OF THE INVENTION

Currently there are no effective screening programs for early detection of ovarian cancer. Blood-based biomarkers fail to detect the disease early enough to have an impact on the survival figures. For this reason, women at high-risk are unanimously advised to undergo prophylactic surgery before the age of 40, since their individual risk cannot be calculated. The inventors describe herein use of a sample obtained from within the gynecologic system i.e. uterine lavage (UtL) fluid, thus tremendously increasing the chance of detecting analytes at minimal concentrations. This assay may be also applicable to plasma/serum samples as well.

The early detection assay provided by the invention, can be implemented to clinical use in the following settings:

General population—women at average risk for ovarian cancer will be offered the screening test after the age of 50. High risk population—women at genetically high risk for developing ovarian cancer will be offered to do the biomarker testing on UtL sample obtained at every routine gynecologic follow-up examination starting at the age of 30. The test will yield either a reassuring result, requiring continuation of the regular follow-up program, or an alarming result indicating further diagnostic tests (pelvic Doppler sonography or exploratory laparoscopy). Alternatively, women at average risk would be referred by a primary care physician to the screening test, which would be performed on plasma, and those women with alarming results would be further referred to a gynecologic consult.

By using machine learning algorithms developed recently by the inventors, a 9-protein diagnostic signature were defined which were used to predict HGOC with 83% sensitivity and 91.6% specificity in an independent validation set of 152 samples. This relatively high specificity was achieved despite significant differences in the clinical characteristics of the discovery and validation cohorts, which result from fluctuate availability of eligible study populations. These properties are far superior to previously reported 40-60% detection rate in previous similar works [12-14]. Of special note, the sample set included four early-stage lesions—three cases of stage IA HGOC and one case of serous tubal intraepithelial carcinoma (STIC), and all were predicted as ‘tumors’. The proteomic signature may be integrated with a genomic test to further increase the predictive power. The selection of proteins to be included in the signature was unbiased. The Differential Expression of these proteins was further validated by comparing mRNA and IHC stains in normal FTE vs HGOC tumors.

As shown in Example 3 herein, the inventors identified the following specific set of signature proteins CLCA4, OVGP1, S100A14, SPRR3, RNASE3, SERPINB5, CLUAP1, CEACAM5 and ENPP3 that are differently expressed in patients diagnosed as suffering from ovarian cancer compared to a control group. The inventors have therefore suggested that the identified signature proteins described herein are suitable as a powerful tool for early diagnosis of ovarian cancer. More specifically, the nine-protein classifier of the invention, based upon proteomic profiling of microvesicles from UtL samples, display 73% sensitivity,64% specificity and NPV=90% which outperform previous results of genomic biomarkers based on gynecological liquid biopsy. Unlike mutation analysis in UtL samples which looks at a negligible percent of cancer cells, proteomics reflects the complexity of a cancer-associated program that captures expressional changes in multiple cell types within the tumor microenvironment, thus can potentially provide a wider array of early-detection biomarkers. Further improvement of the proteomic signature and its predictive power, requires analysis of more early-stage HGOC UtL samples or STICs, however, these samples are inherently exceedingly rare.

The UtL sampling technique that is proposed hereby is a simplified version of the originally reported method (15) which is based on the use of a specialized proprietary catheter. The present technique has the advantage of use of widely-available and inexpensive insemination catheter, making it suitable for routine testing of healthy young women at high risk for HGOC, including nulliparous women. Fundamental parameters for clinical feasibility, such as patient-reported outcomes and physicians-reported workload are favorable, with high compliance of the target population to undergo routine UtL sampling. The relative low cost, simple handing and processing protocols and rapid dissemination of MS platforms in clinical labs, make proteomic testing of UtL liquid biopsies appealing. Specifically, semi-annual monitoring with proteomic assay may be implemented as a measure of reassurance for high risk populations willing to delay RRBSO until after menopause, and thus become practice-changing.

Analysis of the microvesicle fraction of liquid biopsies is a novel proteomic approach, presenting immense opportunities for biomarker discovery in other accessible body fluids for the purpose of early cancer detection.

To consolidate the specificity of the signature proteins to HGOC tissues, the inventors examined their expression in independent tissue specimens, comparing FTE and HGOC, using complementary techniques: RT-PCR and IHC. Confirmatory results were obtained for the tested biomarkers, clearly establishing the aberrant expression of these proteins in HGOC tissues. Ultimately, the genomic and the proteomic approaches, as well as other possible methodologies of liquid biopsy analysis, should be integrated to yield a multi-modality classifier with an adequate sensitivity and specificity to guarantee early detection of HGOC in high-risk populations, and potentially enable personalized risk stratification and delay of RRBSO in women without increased HGOC incidence.

Therefore, in a first aspect, the invention provides a diagnostic method for detecting ovarian cancer in a subject. More specifically, the method of the invention may comprise the steps of: In a first step (a) determining the expression level of at least one biomarker protein in at least one biological sample of said subject, to obtain an expression value for each of said at least one biomarker protein/s. More specifically, the at least one biomarker proteins may be selected from Calcium-activated chloride channel regulator 4 (CLCA4), Oviduct-specific glycoprotein (OVGP1), 5100 calcium binding protein A14 (S100A14), Small proline-rich protein 3 (SPRR3), Eosinophil cationic protein (RNASE3), Serpin Family B Member 5 (SERPINB5), Clusterin-associated protein 1 (CLUAP1), Carcinoembryonic antigen-related cell adhesion molecule 5 (CEACAM5) and Ectonucleotide pyrophosphatase/phosphodiesterase family member 3 (ENPP3) or any combination thereof. In the next step (b), the method of the invention involves determining if the expression value obtained in step (a) for each of the at least one biomarker protein/s is positive or negative with respect to a predetermined standard expression value or alternatively or additionally, to the expression value of said biomarker protein/s in at least one control sample. In some specific embodiments, a result of at least one of (i) a positive expression value of at least one of the SPRR3, SERPINB5, CEACAM5, S100A14 and CLCA4 biomarker protein/s in the tested sample, indicates that the subject belongs to a predetermined population suffering from ovarian cancer; and (ii) a negative expression value of at least one of the OVGP1, CLUAP1, ENPP3 and RNASE3 biomarker protein/s in said sample, indicates that the subject belongs to a predetermined population suffering from ovarian cancer. In other words, the subject may suffers and therefore diagnosed as suffering from ovarian cancer.

It should be understood that determination of a “positive” or alternatively “negative” expression value with respect to a standard value or a control value may involve in some embodiments comparison of the expression value of the examined sample as obtained in step (a), with the expression value obtained for a control sample, or from any established or predetermined expression value (e.g., a standard value) obtained from a known control (either healthy controls or of subjects suffering from ovarian cancer). Thus, in some embodiments, “positive” is meant an expression value that is higher, increased, elevated, overexpressed in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the expression value of a healthy control, any other suitable control or any other predetermined standard. Still further, a “negative” expression value in some embodiments may be a reduced, low, non-existing or lack of expression of a biomarker in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the expression value of a healthy control, any other suitable control or any other predetermined standard.

Thus, in some embodiments, step (b) of the methods of the invention may involves comparing the expression value obtained in step (a) with the expression value of an appropriate control or standard. Wherein the expression value obtained in the examined sample for at least one of SPRR3, SERPINB5, CEACAM5, S100A14 and CLCA4, is “positive”, specifically, higher, overexpressed, elevated when compared to a healthy control, the subject is classified as a subject that carry or has an ovarian cancer. It should be noted that in case of biomarkers that are overexpressed in ovarian cancer, for example, any one of SPRR3, SERPINB5, CEACAM5, S100A14 and CLCA4, a “positive” expression value should be in the range of the expression value of a control patient diagnosed with ovarian cancer, or any other cut off value obtained for a population of ovarian cancer patients. Still further, when the expression value obtained in the examined sample for at least one of OVGP1, CLUAP1, ENPP3 and RNASE3, is determined as “negative”, specifically, higher, overexpressed, elevated when compared to a healthy control, the subject is classified as a subject that carry or has an ovarian cancer. It should be noted that in case of biomarkers that display reduced, low or non-existing expression in ovarian cancer, for example, any one of OVGP1, CLUAP1, ENPP3 and RNASE3, a “negative” expression value should be in the range of the expression value of a control patient diagnosed with ovarian cancer, or any other cut off value obtained for a population of ovarian cancer patients.

It should be noted that the detecting molecules may be provided in a diagnostic composition or in a kit either attached to a solid support or alternatively, in a mixture. Thus, the method of the invention encompasses in certain embodiments also the provision of a composition, kit, solid support or mixture comprising at least one detecting molecule specific for at least one of said biomarker proteins of the invention.

More particularly, the method of the invention may use as diagnostic tool, the expression values of each and any one of the marker proteins described herein below or of any combinations thereof. Specifically, determining the expression values of at least one of CLCA4, OVGP1, S100A14, SPRR3, RNASE3, SERPINB5, CLUAP1, CEACAM5 and ENPP3 proteins may indicate if a subject belongs to a predetermined population suffering from ovarian cancer, or in other words, if the subject should be diagnosed as a subject affected with ovarian cancer.

In some specific embodiments, the biomarker protein of the invention may be the Oviduct-specific glycoprotein (OVGP1) protein. Thus, in certain embodiments, the methods, compositions and kits of the invention may use as a diagnostic tool the expression value of this biomarker either alone or in any combination with any of the biomarker protein/s disclosed by the invention. OVGP1 (or MUC9) as described herein refers to the human OVGP1 (UNITPROT ID: Q12889, Accession number: NP_002548.3). This protein is a mullerian tract specific protein, expressed in the benign cell-of-origin of high grade ovarian cancer and also shown to be elevated in non-serous ovarian tumors [27]. In more specific embodiments, the OVGP1 protein as used herein may comprise the amino acid sequence as denoted by SEQ ID NO. 1 and may be encoded by the nucleic acid sequence as denoted by SEQ ID NO. 2.

In some specific embodiments, the biomarker protein of the invention may be the Small proline-rich protein 3 (SPRR3) protein. Thus, in certain embodiments, the methods, compositions and kits of the invention may use as a diagnostic tool the expression value of this biomarker either alone or in any combination with any of the biomarker protein/s disclosed by the invention. SPRR3 as described herein refers to the human SPRR3 (UNITPROT ID: Q9UBC9, Accession number: AK311823.1). This protein is a cross-linked envelope protein of keratinocytes, but also reported to be over-expressed and involved in the metastatic spread of several cancer types. In more specific embodiments, the SPRR3 protein as used herein may comprise the amino acid sequence as denoted by SEQ ID NO. 3 and may be encoded by the nucleic acid sequence as denoted by SEQ ID NO. 4.

In some specific embodiments, the biomarker protein of the invention may be the Calcium-activated chloride channel regulator 4 (CLCA4) protein. Thus, in certain embodiments, the methods, compositions and kits of the invention may use as a diagnostic tool the expression value of this biomarker either alone or in any combination with any of the biomarker protein/s disclosed by the invention. CLCA4 as described herein refers to the human CLCA4 (UNITPROT ID: Q14CN2, Accession number: NM_012128.3). This protein is involved in mediating calcium-activated chloride conductance, and associated with proliferation and epithelial-to-mesenchymal transformation in several tumor types. In more specific embodiments, the CLCA4 protein as used herein may comprise the amino acid sequence as denoted by SEQ ID NO. 5 and may be encoded by the nucleic acid sequence as denoted by SEQ ID NO. 6.

In some specific embodiments, the biomarker protein of the invention may be the S100 calcium binding protein A14 (S100A14) protein. Thus, in certain embodiments, the methods, compositions and kits of the invention may use as a diagnostic tool the expression value of this biomarker either alone or in any combination with any of the biomarker protein/s disclosed by the invention. S100A14 as described herein refers to the human S100A14 (UNITPROT ID: Q9HCY8, Accession number: NM_020672). This protein is involved in mediating calcium-activated chloride conductance. This protein is a member of the S100 protein family, which is aberrantly expressed in several epithelial cancers. In more specific embodiments, the S100A14 protein as used herein may comprise the amino acid sequence as denoted by SEQ ID NO. 7 and may be encoded by the nucleic acid sequence as denoted by SEQ ID NO. 8.

In some specific embodiments, the biomarker protein of the invention may be the Clusterin-associated protein 1 (CLUAP1) protein. Thus, in certain embodiments, the methods, compositions and kits of the invention may use as a diagnostic tool the expression value of this biomarker either alone or in any combination with any of the biomarker protein/s disclosed by the invention. CLUAP1 as described herein refers to the human CLUAP1 (UNITPROT ID: Q96AJ1, Accession number: NM_015041.2). This protein is required for cilia biogenesis, appears to be a key regulator of hedgehog signaling and up-regulated in several cancer types. In more specific embodiments, the CLUAP1 protein as used herein may comprise the amino acid sequence as denoted by SEQ ID NO. 9 and may be encoded by the nucleic acid sequence as denoted by SEQ ID NO. 10.

In certain embodiments, the biomarker protein of the invention may be the Serpin Family B Member 5 (SERPINB5) protein. Thus, in certain embodiments, the methods, compositions and kits of the invention may use as a diagnostic tool the expression value of this biomarker either alone or in any combination with any of the biomarker protein/s disclosed by the invention. SERPINB5, as used herein, refers to the human SERPINB5 (Accession number: NM_002639). This protein belongs to the serpin (serine protease inhibitor) superfamily. SERPINB5 was originally reported to function as a tumor suppressor gene in epithelial cells, suppressing the ability of cancer cells to invade and metastasize to other tissues. In more specific embodiments, the SERPINB5 protein as used herein may comprise the amino acid sequence as denoted by SEQ ID NO. 11 and may be encoded by the nucleic acid sequence as denoted by SEQ ID NO. 12.

In some specific embodiments, the biomarker protein of the invention may be the Eosinophil cationic protein (RNASE3) protein. Thus, in certain embodiments, the methods, compositions and kits of the invention may use as a diagnostic tool the expression value of this biomarker either alone or in any combination with any of the biomarker protein/s disclosed by the invention. RNASE3 as described herein refers to the human RNASE3 (UNITPROT ID: P12724, Accession number: NP_002926.2). This protein is a Cytotoxin and helminthotoxin with low-efficiency ribonuclease activity. It possesses a wide variety of biological activities, however its role in cancer is unknown. In more specific embodiments, the RNASE3 protein as used herein may comprise the amino acid sequence as denoted by SEQ ID NO. 13 and may be encoded by the nucleic acid sequence as denoted by SEQ ID NO. 14.

In some specific embodiments, the biomarker protein of the invention may be the Carcinoembryonic antigen-related cell adhesion molecule 5 (CEACAM5) protein. Thus, in certain embodiments, the methods, compositions and kits of the invention may use as a diagnostic tool the expression value of this biomarker either alone or in any combination with any of the biomarker protein/s disclosed by the invention. CEACAM5 as described herein refers to the human CEACAM5 (UNITPROT ID: P06731, Accession number: NP_001278413.1). This protein is a cell surface glycoprotein that plays a role in cell adhesion and in intracellular signaling. In more specific embodiments, the CEACAM5 protein as used herein may comprise the amino acid sequence as denoted by SEQ ID NO. 15 and may be encoded by the nucleic acid sequence as denoted by SEQ ID NO. 16.

In some specific embodiments, the biomarker protein of the invention may be the Ectonucleotide pyrophosphatase/phosphodiesterase family member 3 (ENPP3) protein. Thus, in certain embodiments, the methods, compositions and kits of the invention may use as a diagnostic tool the expression value of this biomarker either alone or in any combination with any of the biomarker protein/s disclosed by the invention. ENPP3 as described herein refers to the human ENPP3 (UNITPROT ID: 014638, Accession number: NP_005012.2). This protein cleaves a variety of phosphodiester and phosphosulfate bonds including deoxynucleotides, nucleotide sugars, and NAD. In more specific embodiments, the ENPP3 protein as used herein may comprise the amino acid sequence as denoted by SEQ ID NO. 17 and may be encoded by the nucleic acid sequence as denoted by SEQ ID NO. 18. In yet some further embodiments, any of the 9-signatory biomarkers of the invention specified above, may be combined in some embodiments with any additional biomarker. In some further specific embodiments, such at least one additional biomarker may be any one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3. In some particular embodiments, the method of the invention may use as biomarkers any one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3, either alone, or in combination with any one of at least one of the 9-signatory biomarkers of the invention, specifically, any one of CLCA4, OVGP1, S100A14, SPRR3, RNASE3, SERPINB5, CLUAP1, CEACAM5 and ENPP3. In some particular embodiments, any one of S100A14 and SERPINB5 of the 9-signatory biomarkers of the invention may be combined with at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3.

More particularly, in some specific embodiments, the biomarker protein of the invention may be the Carcinoembryonic Antigen-Related Cell Adhesion Molecule 6 (CEACAM6) protein. Thus, in certain embodiments, the methods, compositions and kits of the invention may use as a diagnostic tool the expression value of this biomarker either alone or in combination with any of the biomarker protein/s disclosed by the invention. CEACAM6 as described herein refers to the human CEACAM6 (Accession number: NM_002483). This protein belongs to the carcinoembryonic antigen (CEA) family whose members are glycosyl phosphatidyl inositol (GPI) anchored cell surface glycoproteins. In more specific embodiments, the CEACAM6 protein as used herein comprises the amino acid sequence as denoted by SEQ ID NO. 19 and may be encoded by the nucleic acid sequence as denoted by SEQ ID NO. 20.

In other specific embodiments the biomarker protein of the invention may be the Galectin-7 (LGALS7) protein. Thus, in certain embodiments, the methods, compositions and kits of the invention may use as a diagnostic tool the expression value of this biomarker either alone or in combination with any of the biomarker protein/s disclosed by the invention. LGALS7 as described herein, refers to the human LGALS7 (Accession number: NM_002307). This protein belongs to a family of beta-galactoside-binding proteins implicated in modulating cell-cell and cell-matrix interactions. In more specific embodiments, the LGALS7 protein as used herein comprises the amino acid sequence as denoted by SEQ ID NO. 21 and may be encoded by the nucleic acid sequence as denoted by SEQ ID NO. 22.

In certain embodiments, the biomarker protein of the invention may be the Branched Chain Amino Acid Transaminase 1 (BCAT1) protein. Thus, in certain embodiments, the methods, compositions and kits of the invention may use as a diagnostic tool the expression value of this biomarker either alone or in combination with any of the biomarker protein/s disclosed by the invention. BCAT1 as described herein, refers to the human BCAT1 (Accession number: NM_001178091). This protein is an enzyme that catalyzes the reversible transamination of branched-chain alpha-keto acids to branched-chain L-amino acids essential for cell growth. In more specific embodiments, the BCAT1 protein as used herein comprises the amino acid sequence as denoted by SEQ ID NO. 23 and may be encoded by the nucleic acid sequence as denoted by SEQ ID NO. 24.

In certain embodiments, the biomarker protein of the invention may be the Adipogenesis regulatory factor (ADIRF) protein. Therefore, in some embodiments, the methods, compositions and kits of the invention may use as a diagnostic tool the expression value of this biomarker either alone or in combination with any of the biomarker protein/s disclosed by the invention. ADIRF as described herein, refers to the human (Accession number: NM_006829). This protein plays a role in fat cell development; promotes adipogenic differentiation and stimulates transcription initiation of master adipogenesis factors. In more specific embodiments, the ADIRF protein as used herein comprises the amino acid sequence as denoted by SEQ ID NO. 25 and may be encoded by the nucleic acid sequence as denoted by SEQ ID NO. 26.

In other specific embodiments, the biomarker protein of the invention may be the Cornulin (CRNN) protein. According to some embodiments, the methods, compositions and kits of the invention may use as a diagnostic tool the expression value of this biomarker either alone or in combination with any of the biomarker protein/s disclosed by the invention. CRNN, as used herein, refers to the human CRNN (Accession number: NM_016190). This protein that is also known as squamous epithelial heat shock protein 53, may play a role in the mucosal/epithelial immune response and epidermal differentiation. In more specific embodiments, the CRNN protein as used herein comprises the amino acid sequence as denoted by SEQ ID NO. 27 and may be encoded by the nucleic acid sequence as denoted by SEQ ID NO.28.

In further embodiments, the biomarker protein of the invention may be the Agrin (AGRN). AGRN herein, refers to the human AGRN (Accession number: NM_198576). Thus, in certain embodiments, the methods, compositions and kits of the invention may use as a diagnostic tool the expression value of this biomarker either alone or in combination with any of the biomarker protein/s disclosed by the invention. The AGRN protein is critical in the development of the neuromuscular junction (NMJ), as identified in mouse knockout studies. In more specific embodiments, the AGRN protein as used herein comprises the amino acid sequence as denoted by SEQ ID NO. 29 and may be encoded by the nucleic acid sequence as denoted by SEQ ID NO.30.

In other embodiments, the biomarker protein of the invention may be the Alcohol dehydrogenase 1B (Class I), Beta Polypeptide (ADH1B) protein. Thus, in certain embodiments, the methods, compositions and kits of the invention may use as a diagnostic tool the expression value of this biomarker either alone or in combination with any of the biomarker protein/s disclosed by the invention. ADH1B, as used herein, refers to the human ADH1B (Accession number: NM_001286650). This protein is a member of an enzymatic family that metabolizes a wide variety of substrates, including ethanol, retinol, other aliphatic alcohols, hydroxysteroids, and lipid peroxidation products. This protein, consisting of several homo- and heterodimers of alpha, beta, and gamma subunits, exhibits high activity for ethanol oxidation and plays a major role in ethanol catabolism. In more specific embodiments, the ADH1B protein as used herein comprises the amino acid sequence as denoted by SEQ ID NO. 31 and may be encoded by the nucleic acid sequence as denoted by SEQ ID NO. 32.

In certain embodiments, the biomarker protein of the invention may be the Cadherin-1 (CDH1) protein. In some embodiments, the methods, compositions and kits of the invention may use as a diagnostic tool the expression value of this biomarker either alone or in combination with any of the biomarker protein/s disclosed by the invention. CDH1 as described herein, refers to the human CDH1 (Accession number: NM_004360). This protein is also known as CAM 120/80 or epithelial cadherin (E-cadherin) or uvomorulin and is a classical member of the cadherin superfamily. It is a calcium-dependent cell-cell adhesion glycoprotein composed of five extracellular cadherin repeats, a transmembrane region, and a highly conserved cytoplasmic tail. In more specific embodiments, the CDH1 protein as used herein comprises the amino acid sequence as denoted by SEQ ID NO. 33 and may be encoded by the nucleic acid sequence as denoted by SEQ ID NO. 34.

In further embodiments, the biomarker protein of the invention may be the Glutamate-ammonia ligase (GLUL) protein. In certain embodiments, the methods, compositions and kits of the invention may use as a diagnostic tool the expression value of this biomarker either alone or in combination with any of the biomarker protein/s disclosed by the invention. GLUL as described herein, refers to the human GLUL (Accession number: NM_002065). This protein belongs to the glutamine synthetase family. It catalyzes the synthesis of glutamine from glutamate and ammonia in an ATP-dependent reaction. This protein plays a role in ammonia and glutamate detoxification, acid-base homeostasis, cell signaling, and cell proliferation. In more specific embodiments, the GLUL protein as used herein comprises the amino acid sequence as denoted by SEQ ID NO. 35 and may be encoded by the nucleic acid sequence as denoted by SEQ ID NO. 36.

In further embodiments, the biomarker protein of the invention may be the Thymus cell surface antigen 1 (THY1) protein. It should be noted that the methods, compositions and kits of the invention may use as a diagnostic tool the expression value of this biomarker either alone or in combination with any of the biomarker protein/s disclosed by the invention. THY1 as described herein, refers to the human THY1 (Accession number: NM_006288). This protein is a heavily N-glycosylated, glycophosphatidylinositol (GPI) anchored conserved cell surface protein with a single V-like immunoglobulin domain, originally discovered as a thymocyte antigen. In more specific embodiments, the THY1 protein as used herein comprises the amino acid sequence as denoted by SEQ ID NO. 37 and may be encoded by the nucleic acid sequence as denoted by SEQ ID NO. 38.

In other embodiment, the biomarker protein of the invention may be the Glutaredoxin-3 (GLRX3) protein. Thus, in certain embodiments, the methods, compositions and kits of the invention may use as a diagnostic tool the expression value of this biomarker either alone or in combination with any of the biomarker protein/s disclosed by the invention. GLRX3, as used herein, refers to the human GLRX3 (Accession number: NM_001199868). This protein is a member of the glutaredoxin family. Glutaredoxins are oxidoreductase enzymes that reduce a variety of substrates using glutathione as a cofactor. The encoded protein binds to and modulates the function of protein kinase C theta. In more specific embodiments, the GLRX3 protein as used herein comprises the amino acid sequence as denoted by SEQ ID NO. 39 and may be encoded by the nucleic acid sequence as denoted by SEQ ID NO. 40.

In some embodiments, the biomarker protein of the invention may be the Versican (VCAN) protein. In some embodiments, the methods, compositions and kits of the invention may use as a diagnostic tool the expression value of this biomarker either alone or in combination with any of the biomarker protein/s disclosed by the invention. VCAN as described herein, refers to the human VCAN (Accession number: NM_001164097). This protein is a member of the aggrecan/versican proteoglycan family. The protein encoded is a large chondroitin sulfate proteoglycan and is a major component of the extracellular matrix. This protein is involved in cell adhesion, proliferation, migration and angiogenesis and plays a central role in tissue morphogenesis and maintenance. In more specific embodiments, the VCAN protein as used herein comprises the amino acid sequence as denoted by SEQ ID NO. 41 and may be encoded by the nucleic acid sequence as denoted by SEQ ID NO.42.

In some other embodiments, the biomarker protein of the invention may be the Carboxypeptidase M (CPM) protein. Thus, in certain embodiments, the methods, compositions and kits of the invention may use as a diagnostic tool the expression value of this biomarker either alone or in combination with any of the biomarker protein/s disclosed by the invention. CPM as used herein, refers to the human CPM (Accession number: NM_001874). This protein is a membrane-bound arginine/lysine carboxypeptidase. Its expression is associated with monocyte to macrophage differentiation. This encoded protein contains hydrophobic regions at the amino and carboxy termini and has 6 potential asparagine-linked glycosylation sites. In more specific embodiments, the CPM protein as used herein comprises the amino acid sequence as denoted by SEQ ID NO. 43 and may be encoded by the nucleic acid sequence as denoted by SEQ ID NO. 44.

In certain embodiments, the biomarker protein of the invention may be the Hematopoietic Progenitor Cell Antigen, also known as Cluster of Differentiation 34 (CD34) protein. In certain embodiments, the methods, compositions and kits of the invention may use as a diagnostic tool the expression value of this biomarker either alone or in combination with any of the biomarker protein/s disclosed by the invention. CD34 as herein, refers to the human CD34 (Accession number: NM_001773). This protein is an important adhesion molecule and is required for T cells to enter lymph nodes. It is expressed on lymph node endothelia, whereas the L-selectin to which it binds is on the T cell. In more specific embodiments, the CD34 protein as used herein comprises the amino acid sequence as denoted by SEQ ID NO. 45 and may be encoded by the nucleic acid sequence as denoted by SEQ ID NO. 46.

In some further embodiments, the biomarker protein of the invention may be the Cluster of Differentiation 109 (CD109) protein. Thus, in certain embodiments, the methods, compositions and kits of the invention may use as a diagnostic tool the expression value of this biomarker either alone or in combination with any of the biomarker protein/s disclosed by the invention. CD109 as described herein, refers to the human CD109 (Accession number: NM_133493). This protein is a GPI-linked cell surface antigen expressed by T-cell lines, activated T lymphoblasts, endothelial cells, and activated platelets. In addition, the platelet-specific Gov antigen system, implicated in refractoriness to platelet transfusion, neonatal alloimmune thrombocytopenia, and posttransfusion purpura, is carried by CD109. In more specific embodiments, the CD109 protein as used herein comprises the amino acid sequence as denoted by SEQ ID NO. 47 and may be encoded by the nucleic acid sequence as denoted by SEQ ID NO. 48.

In certain embodiments, the biomarker protein of the invention may be the Intelectin-1 (ITLN1) protein. In certain embodiments, the methods, compositions and kits of the invention may use as a diagnostic tool the expression value of this biomarker either alone or in combination with any of the biomarker protein/s disclosed by the invention. ITLN1, as used herein, refers to the human ITLN1 (Accession number: NM_017625). This protein functions both as a receptor for bacterial arabinogalactans and for lactoferrin. Having conserved ligand binding site residues, both human and mouse intelectin-1 bind the exocyclic vicinal diol of carbohydrate ligands such as galactofuranose. In more specific embodiments, the ITLN1 protein as used herein comprises the amino acid sequence as denoted by SEQ ID NO. 49 and may be encoded by the nucleic acid sequence as denoted by SEQ ID NO. 50.

In some other embodiments, the biomarker protein of the invention may be the Complement C1r Subcomponent Like (C1RL) protein. In some embodiments, the methods, compositions and kits of the invention may use as a diagnostic tool the expression value of this biomarker either alone or in combination with any of the biomarker protein/s disclosed by the invention. C1RL, as used herein, refers to the human C1RL (Accession number: NM_001297642). This protein mediates the proteolytic cleavage of HP/haptoglobin in the endoplasmic reticulum. In more specific embodiments, the C1RL protein as used herein comprises the amino acid sequence as denoted by SEQ ID NO. 51 and may be encoded by the nucleic acid sequence as denoted by SEQ ID NO. 52.

In further embodiments, the biomarker protein of the invention may be the Engulfment Adaptor PTB Domain Containing 1 (GULP1) protein. Thus, in certain embodiments, the methods, compositions and kits of the invention may use as a diagnostic tool the expression value of this biomarker either alone or in combination with any of the biomarker protein/s disclosed by the invention. GULP1 as described herein, refers to the human (Accession number: NM_001252668). This protein is an adapter protein necessary for the engulfment of apoptotic cells by phagocytes. In more specific embodiments, the GULP1 protein as used herein comprises the amino acid sequence as denoted by SEQ ID NO. 53 and may be encoded by the nucleic acid sequence as denoted by SEQ ID NO. 54.

In certain embodiments, the biomarker protein of the invention may be the N-Myc Downstream-Regulated Gene 3 (NDRG3) protein. In certain embodiments, the methods, compositions and kits of the invention may use as a diagnostic tool the expression value of this biomarker either alone or in combination with any of the biomarker protein/s disclosed by the invention. NDRG3, as used herein, refers to the human NDRG3 (Accession number: NM_032013. This protein is implicated in several pathways such as apoptosis, autophagy and angiogenesis. In more specific embodiments, the NDRG3 protein as used herein comprises the amino acid sequence as denoted by SEQ ID NO. 55 and may be encoded by the nucleic acid sequence as denoted by SEQ ID NO. 56.

In some embodiments, the expression value of at least one biomarker protein, at times at least two proteins, at times at least three proteins, at times at least four proteins, at times at least five proteins, at times at least six proteins, at times at least seven proteins, at times at least eight proteins, at times at least nine proteins, of any one of CLCA4, OVGP1, S100A14, SPRR3, RNASE3, SERPINB5, CLUAP1, CEACAM5 and ENPP3 may be determined.

In certain embodiments, the methods of the invention may involve determination of the expression level of all CLCA4, OVGP1, S100A14, SPRR3, RNASE3, SERPINB5, CLUAP1, CEACAM5 and ENPP3 biomarker proteins.

It should be noted that the biomarker proteins of the invention are disclosed in Table 4 herein after.

According to some embodiments, step (a) of the method of the invention may involve determining the expression level of at least two biomarker proteins in at least one biological sample of said subject, to obtain an expression value for each of said at least two biomarker protein/s. It should be noted that at least two biomarker proteins may be selected from CLCA4, OVGP1, S100A14, SPRR3, RNASE3, SERPINB5, CLUAP1, CEACAM5 and ENPP3. In some particular and non-limiting embodiments of the invention, such at least two of CLCA4, OVGP1, S100A14, SPRR3, RNASE3, SERPINB5, CLUAP1, CEACAM5 and ENPP3 biomarker protein/s may comprise CLCA4 and S100A14. It should be appreciated that in some embodiments, the three biomarker proteins may further comprise at least one, at least two, at least three, at least four, at least five, at least six, at least seven, of the OVGP1, SPRR3, RNASE3, SERPINB5, CLUAP1, CEACAM5 and ENPP3 biomarker proteins of the invention. According to some embodiments, step (a) of the method of the invention may involve determining the expression level of at least two biomarker proteins in at least one biological sample of said subject, to obtain an expression value for each of said at least two biomarker protein/s. It should be noted that at least two biomarker proteins may be selected from CLCA4, OVGP1, S100A14, SPRR3, RNASE3, SERPINB5, CLUAP1, CEACAM5 and ENPP3.

In some particular and non-limiting embodiments of the invention, such at least two of CLCA4, OVGP1, S100A14, SPRR3, RNASE3, SERPINB5, CLUAP1, CEACAM5 and ENPP3 biomarker protein/s may comprise S100A14 and SERPINB5. It should be appreciated that in some embodiments, the threat least two biomarker proteins may further comprise at least one, at least two, at least three, at least four, at least five, at least six, at least seven, of the CLCA4, OVGP1, SPRR3, RNASE3, CLUAP1, CEACAM5 and ENPP3 biomarker proteins of the invention. In yet some further specific and non-limiting embodiments, the method of the invention (as well as any compositions and kits thereof) may use said at least two biomarker protein and further, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3.According to some embodiments, step (a) of the method of the invention may involve determining the expression level of at least three biomarker proteins in at least one biological sample of said subject, to obtain an expression value for each of said at least three biomarker protein/s. It should be noted that at least three biomarker proteins may be selected from CLCA4, OVGP1, S100A14, SPRR3, RNASE3, SERPINB5, CLUAP1, CEACAM5 and ENPP3.

In some particular and non-limiting embodiments of the invention, such at least three of CLCA4, OVGP1, S100A14, SPRR3, RNASE3, SERPINB5, CLUAP1, CEACAM5 and ENPP3biomarker protein/s may comprise CLCA4, OVGP1 and S100A14. It should be appreciated that in yet some further embodiments, the at least three biomarker proteins may further comprise at least one, at least two, at least three, at least four, at least five, at least six, of the SPRR3, RNASE3, SERPINB5, CLUAP1, CEACAM5 and ENPP3 biomarker proteins of the invention. In yet some further specific and non-limiting embodiments, the method of the invention (as well as any compositions and kits thereof) may use said at least three biomarker protein and in addition, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3.

In certain embodiments, step (a) of the method of the invention may involve determining the expression level of at least four biomarker proteins in at least one biological sample of said subject, to obtain an expression value for each of said at least four biomarker protein/s. More specifically, these at least four biomarker proteins may be selected from CLCA4, OVGP1, S100A14, SPRR3, RNASE3, SERPINB5, CLUAP1, CEACAM5 and ENPP3.

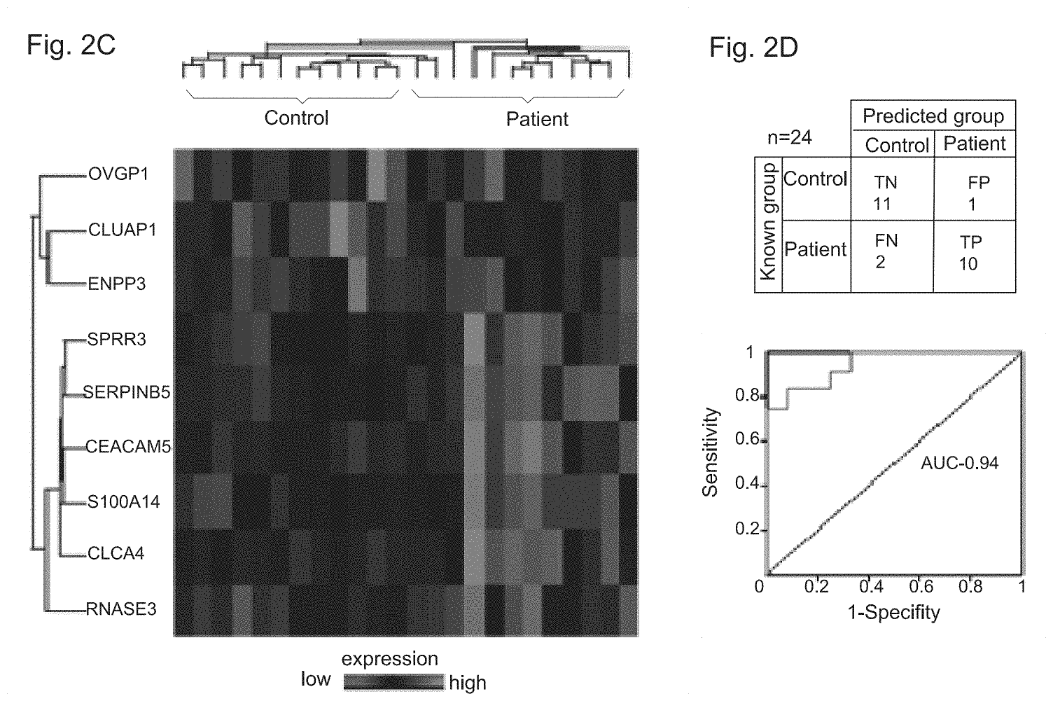

As shown in the Anova and RFE-SVM analysis presented in Example 2 (FIG. 2B), in some particular and non-limiting embodiments of the invention, such at least four of CLCA4, OVGP1, S100A14, SPRR3, RNASE3, SERPINB5, CLUAP1, CEACAM5 and ENPP3 biomarker protein/s may comprise S100A14, CLCA4, CLUAP1 and CEACAM5. It should be appreciated that in some embodiments, the four biomarker proteins may further comprise at least one, at least two, at least three, at least four, at least five of the OVGP1, SPRR3, RNASE3, SERPINB5, and ENPP3 biomarker proteins of the invention.

As shown in the Anova and SVM analysis presented in Example 2 (FIG. 2B), in some particular and non-limiting embodiments of the invention, such at least four of CLCA4, OVGP1, S100A14, SPRR3, RNASE3, SERPINB5, CLUAP1, CEACAM5 and ENPP3 biomarker protein/s may comprise S100A14, CLCA4, SPRR3, SERPINB5. It should be appreciated that in some embodiments, the four biomarker proteins may further comprise at least one, at least two, at least three, at least four, at least five, of the OVGP1, RNASE3, CLUAP1, CEACAM5 and ENPP3 biomarker proteins of the invention. In yet some further specific and non-limiting embodiments, the method of the invention (as well as any compositions and kits thereof) may use said at least four biomarker protein and in addition, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3.

As shown in the RFE-SVM and SVM analysis presented in Example 2 (FIG. 2B), in some particular and non-limiting embodiments of the invention, such at least five of CLCA4, OVGP1, S100A14, SPRR3, RNASE3, SERPINB5, CLUAP1, CEACAM5 and ENPP3 biomarker protein/s may comprise S100A14, CLCA4, OVGP1, ENPP3 and RNASE3. It should be appreciated that in some embodiments, the at least five biomarker proteins may further comprise at least one, at least two, at least three, at least four of the SPRR3, SERPINB5, CLUAP1 and CEACAM5 biomarker proteins of the invention. In yet some further specific and non-limiting embodiments, the method of the invention (as well as any compositions and kits thereof) may use said at least five biomarker protein and in addition, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3.

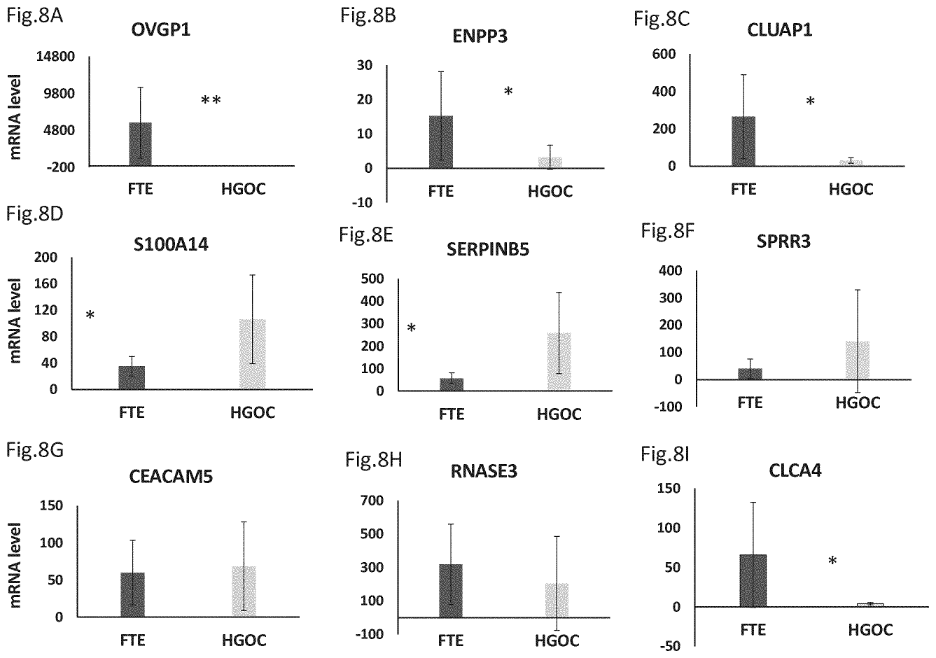

In yet some further alternative embodiments, the method of the invention may involve in step (a) determination of the expression level of at least six biomarker proteins. In some particular and non-limiting embodiments of the invention, such at least six of CLCA4, OVGP1, S100A14, SPRR3, RNASE3, SERPINB5, CLUAP1, CEACAM5 and ENPP3 biomarker protein/s may comprise OVGP1, CLCA4, S100A14, CLUAP1, SERPINB5 and ENPP3, as shown by FIG. 8. It should be appreciated that in some embodiments, the six biomarker proteins may further comprise at least one, at least two, at least three, of the SPRR3, RNASE3 and CEACAM5 biomarker proteins of the invention.

Still in yet some further embodiments, the method of the invention may involve in step (a) determination of the expression level of at least six biomarker proteins. In some particular and non-limiting embodiments of the invention, such at least six of CLCA4, OVGP1, S100A14, SPRR3, RNASE3, SERPINB5, CLUAP1, CEACAM5 and ENPP3 biomarker protein/s may comprise SERPINB5, S100A14, OVGP1, CLCA4, CLUAP1 and CEACAM5. It should be appreciated that in some embodiments, the six biomarker proteins may further comprise at least one, at least two, at least three, of the SPRR3, RNASE3, and ENPP3 biomarker proteins of the invention. In yet some further specific and non-limiting embodiments, the method of the invention (as well as any compositions and kits thereof) may use said at least six biomarker protein and in addition, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3.

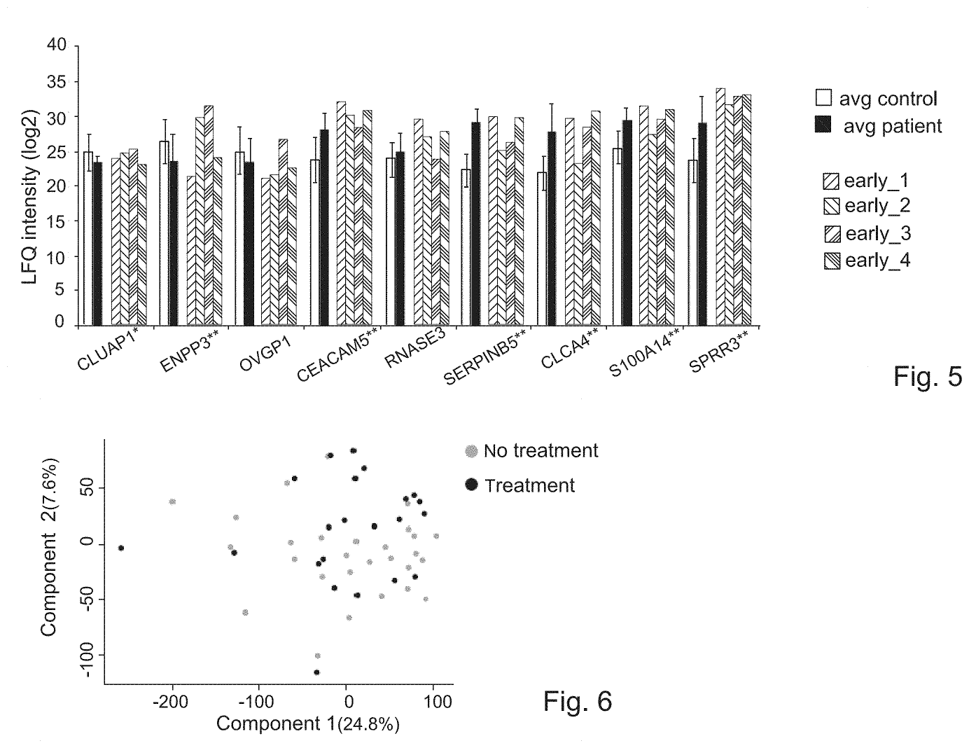

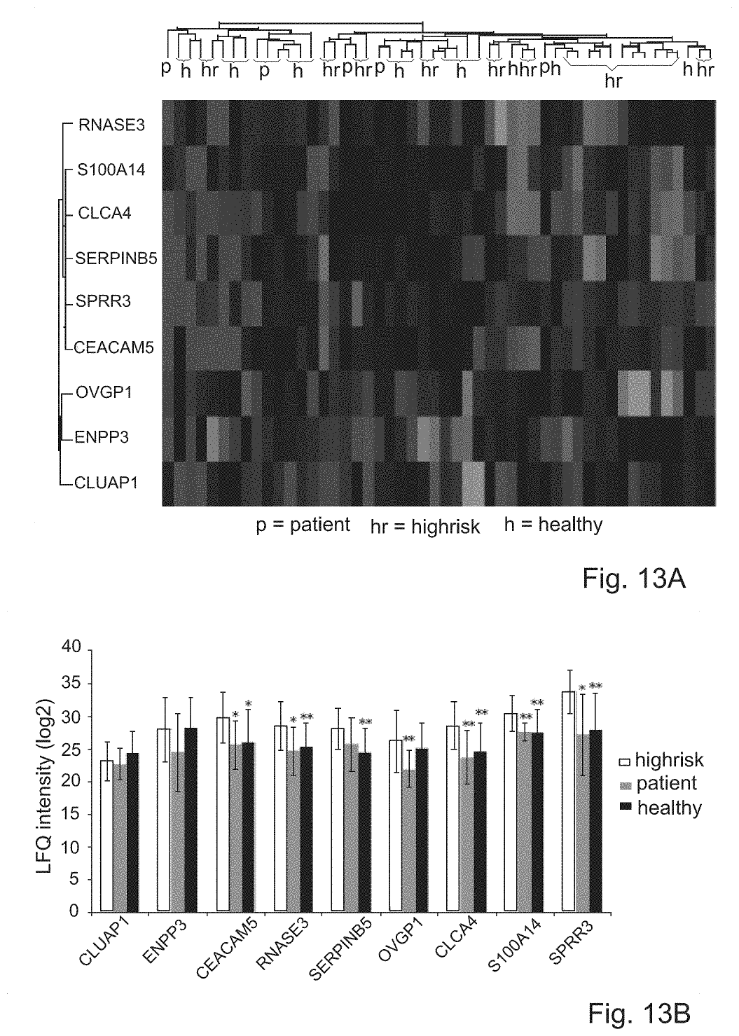

In some particular and non-limiting embodiments of the invention, the method of the invention may involve in step (a) determination of the expression level of at least seven biomarker proteins. In some particular and non-limiting embodiments of the invention, such at least seven of CLCA4, OVGP1, S100A14, SPRR3, RNASE3, SERPINB5, CLUAP1, CEACAM5 and ENPP3 biomarker protein/s may comprise CEACAM5, RNASE3, SERPINB5, OVGP1, CLCA4, S100A14, SPRR3, as also demonstrated by FIG. 13 of Example 7. It should be appreciated that in some embodiments, the seven biomarker proteins may further comprise at least one, at least two of the ENPP3 and CLUAP1. Still further, as shown by FIG. 5, the at least seven biomarker proteins may comprise CLCA4, S100A14, SPRR3, SERPINB5, CLUAP1, CEACAM5 and ENPP3. In yet some further embodiments, the seven biomarker proteins may further comprise at least one or at least two of OVGP1 and RNASE3. In yet some further specific and non-limiting embodiments, the method of the invention (as well as any compositions and kits thereof) may use said at least seven biomarker protein and in addition, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3.

In some particular and non-limiting embodiments of the invention, the method of the invention may involve in step (a) determination of the expression level of at least eight biomarker proteins. In some particular and non-limiting embodiments of the invention, such at least eight of CLCA4, OVGP1, S100A14, SPRR3, RNASE3, SERPINB5, CLUAP1, CEACAM5 and ENPP3 biomarker protein/s may comprise CLCA4, OVGP1, S100A14, SPRR3, RNASE3, SERPINB5, CLUAP1 and CEACAM5. In yet some further specific and non-limiting embodiments, the method of the invention (as well as any compositions and kits thereof) may use said at least eight biomarker protein and in addition, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3.

In certain embodiments, the method as well as the composition and kit of the invention may provide and use detecting molecules specific for at least one, at least two, at least three, at least four, at least five, at least six, at least seven, at least eight or all nine biomarkers of Table 4 and further, detecting molecule/s specific for at least one additional biomarker protein. It should be noted that each detecting molecule is specific for one biomarker. In some embodiments, the method as well as the kits of the invention described herein after may provide and use further detecting molecules specific for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200, 250, 300, 350, 400, 450 and 500 at the most, additional biomarker proteins. In some specific and non-limiting embodiments, the methods, compositions and kits of the invention may provide and use in addition to detecting molecules specific for at least one of the biomarkers disclosed in Table 4, also at least one detecting molecule specific for at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1, GLRX3, PAFAH1B2, GPC4, CKB, BPI, GSTT1, SET, ENPP1, MPDZ, ALDH1L1, IGFBP4, SFRP1. In some specific embodiments platelet activating factor acetylhydrolase 1b catalytic subunit 2 (PAFAH1B2), as used herein is disclosed by GenBank accession no. NM_002572. In yet some further embodiment glypican 4 (GPC4) as used herein is disclosed by GenBank accession no. NM_001448. Still further, in some embodiments, creatine kinase B (CKB) as used herein is disclosed by GenBank accession no. NM_001823. In certain embodiments bactericidal/permeability-increasing protein (BPI), as used herein is disclosed by GenBank accession no. NM_001725. In some embodiments, glutathione S-transferase theta 1 (GSTT1), as used herein is disclosed by GenBank accession no. NM_000853. In yet some further embodiments, SET nuclear proto-oncogene (SET) as used herein is disclosed by GenBank accession no. NM_003011. Still further, ectonucleotide pyrophosphatase/phosphodiesterase 1 (ENPP1), as used herein is disclosed by GenBank accession no. NM_006208. In further embodiments multiple PDZ domain crumbs cell polarity complex component (MPDZ), as used herein is disclosed by GenBank accession no. NM_003829. It should be noted that in some embodiments aldehyde dehydrogenase 1 family member L1 (ALDH1L1), as used herein is disclosed by GenBank accession no. NM_012190. Still further, in some embodiments, insulin like growth factor binding protein 4 (IGFBP4), as used herein is disclosed by GenBank accession no. NM_001552. In yet some further embodiments, secreted frizzled related protein 1 (SFRP1), as used herein is disclosed by GenBank accession no. NM_003012.

In some embodiments, the methods, as well as the compositions and kits of the invention may provide and use detecting molecules specific for at least one additional biomarker protein and at most, 499 additional marker protein/s. In some specific embodiments, the methods and kit/s of the invention may provide and use detecting molecules specific for at least one of the biomarker proteins of Table 4, and detecting molecules specific for at least one additional biomarkers, provided that detecting molecules specific for 100, 150, 200, 250, 300, 350, 384, 400, 450 and 500 at the most biomarker proteins are used.

In yet some further embodiments, it should be understood that the methods of the invention as well as the compositions and kits described herein after, may involve the determination of the expression levels of the biomarker proteins of the invention and/or the use of detecting molecules specific for said biomarker proteins. Specifically, at least one, at least two, at least three, at least four, at least five, at least six, at least seven, at least eight, at least nine, of the biomarker protein/s of the invention that may further comprise any additional biomarker proteins or control reference protein provided that 500 at the most biomarker proteins and control reference proteins are used. In yet some further specific and non-limiting embodiments, the method of the invention (as well as any compositions and kits thereof) may use said at least one biomarker protein of the 9-signatory biomarkers of the invention and in addition, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3. In some embodiments, the at least one, at least two, at least three, at least four, at least five, at least six, at least seven, at least eight, at least nine, of the biomarker protein/s of the invention may form at least about 10%, 15%, 20%, 25%, 30%, 35%, 40%, 45%, 50%, 55%, 60%, 65%, 70%, 75%, 80%, 85%, 90%, 95% or 100% of the biomarker proteins determined by the methods of the invention. In yet some further embodiments, the detecting molecules specific for at least one, at least two, at least three, at least four, at least five, at least six, at least seven, at least eight, at least nine of the biomarker protein/s of the invention, that are used by the methods of the invention and comprised within any of the compositions and kits of the invention may form at least 10%, 15%, 20%, 25%, 30%, 35%, 40%, 45%, 50%, 55%, 60%, 65%, 70%, 75%, 80%, 85%, 90%, 95% or 100% of detecting molecules used in accordance with the invention. It should be appreciated that for each of the selected biomarker proteins at least one detecting molecules may be used. In case more than one detecting molecule is used for a certain biomarker protein, such detecting molecules may be either identical or different.

As described herein below, MS analysis showed that 5 proteins were found to be up-regulated in HGOC patients, whereas 4 proteins were up-regulated in controls as detailed in Example 3. It is suggested by the inventors that this 9-protein signature described above, or any of the subgroup specified herein, specifically, at least two, at least three, at least four, at least five, at least six, at least seven, at least eight or at least nine biomarker proteins, may enable early detection of ovarian cancer. The inventors envision that this signature may be implemented into clinical applications as established herein, to determine presence of ovarian cancer already at an early stage thereby potentially increasing survival of HGOC patients but also limiting the need of risk-reducing bilateral salpingo oophorectomy (RRBSO) in high-risk population.

The term “cancer” is used herein interchangeably with the term “tumor” and denotes a mass of tissue found in or on the body that is made up of abnormal cells. As used herein, the term “ovarian cancer” is used herein interchangeably with the term “fallopian tube cancer” or “primary peritoneal cancer” referring to a cancer that develops from ovary tissue, fallopian tube tissue or from the peritoneal lining tissue.

Early symptoms can include bloating, abdominopelvic pain, and pain in the side. The most typical symptoms of ovarian cancer include bloating, abdominal or pelvic pain or discomfort, back pain, irregular menstruation or postmenopausal vaginal bleeding, pain or bleeding after or during sexual intercourse, difficulty eating, loss of appetite, fatigue, diarrhea, indigestion, heartburn, constipation, nausea, early satiety, and possibly urinary symptoms (including frequent urination and urgent urination); typically these symptoms are caused by a mass pressing on the other abdominopelvic organs or from metastases.

The most common type of ovarian cancer, comprising more than 95% of cases, is epithelial ovarian carcinoma. These tumors are believed to start in the cells covering the ovaries, and a large proportion may form at end of the fallopian tubes. Less common types of ovarian cancer include germ cell tumors and sex cord stromal tumors.

It must be appreciated that the methods, compositions and kits of the invention may be applicable for invasive as well as non-invasive ovarian carcinoma. When referring to “non-invasive” cancer it should be noted as a cancer that do not grow into or invade normal tissues within or beyond the primary location, for example the ovary or the fallopian tube.

When referring to “invasive cancers” it should be noted as cancer that invades and grows in normal, healthy tissues to form metastasis.

As used herein the term “metastatic cancer” or “metastatic status” refers to a cancer that has spread from the place where it first started to another place in the body. Such a tumor formed by metastatic cancer cells is called a metastatic tumor or a metastasis.

Metastasis in ovarian cancer is very common in the abdomen, and occurs via exfoliation, where cancer cells burst through the ovarian capsule and are able to move freely throughout the peritoneal cavity. Ovarian cancer metastases usually grow on the surface of organs rather than the inside; they are also common on the omentum and the peritoneal lining. Cancer cells can also travel through the lymphatic system and metastasize to lymph nodes connected to the ovaries via blood vessels; i.e. the lymph nodes along the infundibulo-pelvic ligament, the broad ligament, and the round ligament. The most commonly affected groups include the paraaortic, hypogastric, external iliac, obturator, and inguinal lymph nodes. In most cases, ovarian cancer does not metastasize to the liver, lung, brain, or kidneys at time of diagnosis; this differentiates ovarian cancer from many other forms of cancer.

Ovarian cancers are classified according to the microscopic appearance of their structures (histology or histopathology). It must be understood that the methods, compositions and kits of the invention may be applicable for the diagnosis of ovarian carcinoma of any of histological subtypes specified herein after.

Surface epithelial-stromal tumor, also known as ovarian epithelial carcinoma, is the most common type of ovarian cancer, representing approximately 90% of ovarian cancers. It includes serous tumor, endometrioid tumor, clear cell tumor, and mucinous cystadenocarcinoma. Less common tumors are malignant Brenner tumor and transitional cell carcinoma of the ovary. Low-grade serous carcinoma is less aggressive than high-grade serous carcinomas, though it does not typically respond well to chemotherapy or hormonal treatments.

About two-thirds of women with epithelial ovarian carcinoma, are diagnosed with serous

carcinoma. Small-cell ovarian carcinoma is rare and aggressive, with two main subtypes: hypercalcemic and pulmonary. It is typically fatal within 2 years of diagnosis. Hypercalcemic small cell ovarian carcinoma overwhelmingly affects those in their 20s, causes high blood calcium levels, and affects one ovary. Pulmonary small cell ovarian cancer usually affects both ovaries of older women and looks like oat-cell carcinoma of the lung.

Primary peritoneal carcinoma develops from the peritoneum. It can develop even after the ovaries have been removed and may appear similar to mesothelioma.

Clear-cell ovarian carcinomas may be related to endometriosis. Clear-cell adenocarcinomas are histopathologically similar to other clear cell carcinomas, with clear cells and hobnail cells. They represent approximately 5-10% of epithelial ovarian cancers and are associated with endometriosis in the pelvic cavity.

Endometrioid adenocarcinomas make up approximately 15-20% of epithelial ovarian cancers. These tumors frequently co-occur with endometriosis or endometrial cancer.

Mixed mullerian tumors make up less than 1% of ovarian cancer. They have epithelial and mesenchymal cells visible.

Mucinous tumors include mucinous adenocarcinoma and mucinous cystadenocarcinoma. Mucinous adenocarcinomas make up 5-10% of epithelial ovarian cancers. Histologically, they are similar to intestinal or cervical adenocarcinomas, and are often actually metastases of appendiceal or colon cancers.

Pseudomyxoma peritonei refers to a collection of encapsulated mucous or gelatinous material in the abdominopelvic cavity, which is very rarely caused by a primary mucinous ovarian tumor.

Undifferentiated cancers—those where the cell type cannot be determined—make up about 10% of epithelial ovarian cancers. When examined under the microscope, these tumors have very abnormal cells that are arranged in clumps or sheets.

Malignant Brenner tumors are rare. Histologically, they have dense fibrous stroma with areas of transitional epithelium, and some squamous differentiation. To be classified as a malignant Brenner tumor, it must have Brenner tumor foci and transitional cell carcinoma. The transitional cell carcinoma component is typically poorly differentiated and resembles urinary tract cancer. Transitional cell carcinomas represent less than 5% of ovarian cancers. Histologically, they appear similar to bladder carcinoma. The prognosis is intermediate—better than most epithelial cancers but worse than malignant Brenner tumors.

Sex cord-stromal tumor, including estrogen-producing granulosa cell tumor, the benign thecoma, and virilizing Sertoli-Leydig cell tumor or arrhenoblastoma, accounts for 7% of ovarian cancers. They occur most frequently in women between 50 and 69 years of age, but can occur in women of any age, including young girls. They are not typically aggressive and are usually unilateral; they are therefore usually treated with surgery alone. Sex cord-stromal tumors are the main hormone-producing ovarian tumors. Granulosa cell tumors are the most common sex-cord stromal tumors, making up 70% of cases, and are divided into two histologic subtypes: adult granulosa cell tumors, which develop in women over 50, and juvenile granulosa tumors, which develop before puberty or before the age of 30. Both develop in the ovarian follicle from a population of cells that surrounds germinal cells.

Germ cell tumors of the ovary develop from the ovarian germ cells. Germ cell tumor accounts for about 30% of ovarian tumors, but only 5% of ovarian cancers, because most germ-cell tumors are teratomas and most teratomas are benign. Malignant teratomas tend to occur in older women, when one of the germ layers in the tumor develops into a squamous cell carcinoma. Germ-cell tumors tend to occur in young women (20s-30s) and girls, making up 70% of the ovarian cancer seen in that age group. Germ-cell tumors can include dysgerminomas, teratomas, yolk sac tumors/endodermal sinus tumors, and choriocarcinomas, when they arise in the ovary. Some germ-cell tumors have an isochromosome 12, where one arm of chromosome 12 is deleted and replaced with a duplicate of the other.

Dysgerminoma accounts for 35% of ovarian cancer in young women and is the most likely germ cell tumor to metastasize to the lymph nodes; nodal metastases occur in 25-30% of cases. These tumors may have mutations in the KIT gene, a mutation known for its role in gastrointestinal stromal tumor. People with an XY karyotype and ovaries (gonadal dysgenesis) or an X,0 karyotype and ovaries (Turner syndrome) who develop a unilateral dysgerminoma are at risk for a gonadoblastoma in the other ovary, and in this case, both ovaries are usually removed when a unilateral dysgerminoma is discovered to avoid the risk of another malignant tumor. Gonadoblastomas in people with Swyer or Turner syndrome become malignant in approximately 40% of cases. However, in general, dysgerminomas are bilateral 10-20% of the time. Choriocarcinoma can occur as a primary ovarian tumor developing from a germ cell, though it is usually a gestational disease that metastasizes to the ovary. Primary ovarian choriocarcinoma has a poor prognosis and can occur without a pregnancy. They produce high levels of hCG and can cause early puberty in children or menometrorrhagia (irregular, heavy menstruation) after menarche.

Immature, or solid, teratomas are the most common type of ovarian germ cell tumor, making up 40-50% of cases. Teratomas are characterized by the presence of disorganized tissues arising from all three embryonic germ layers: ectoderm, mesoderm, and endoderm; immature teratomas also have undifferentiated stem cells that make them more malignant than mature teratomas (dermoid cysts). The different tissues are visible on gross pathology and often include bone, cartilage, hair, mucus, or sebum, but these tissues are not visible from the outside, which appears to be a solid mass with lobes and cysts.

Mature teratomas, or dermoid cysts, are rare tumors consisting of mostly benign tissue that develop after menopause. The tumors consist of disorganized tissue with nodules of malignant tissue, which can be of various types. The most common malignancy is squamous cell carcinoma, but adenocarcinoma, basal-cell carcinoma, carcinoid tumor, neuroectodermal tumor, malignant melanoma, sarcoma, sebaceous tumor, and struma ovarii can also be part of the dermoid cyst.

Yolk sac tumors, formerly called endodermal sinus tumors, make up approximately 10-20% of ovarian germ cell malignancies, and have the worst prognosis of all ovarian germ cell tumors. They occur both before menarche (in one-third of cases) and after menarche (the remaining two-thirds of cases). Half of people with yolk sac tumors are diagnosed in stage I. Typically, they are unilateral until metastasis, which occurs within the peritoneal cavity and via the bloodstream to the lungs. Yolk sac tumors grow quickly and recur easily, and are not easily treatable once they have recurred.

Embryonal carcinomas, a rare tumor type usually found in mixed tumors, develop directly from germ cells but are not terminally differentiated; in rare cases they may develop in dysgenetic gonads. They can develop further into a variety of other neoplasms, including choriocarcinoma, yolk sac tumor, and teratoma. They occur in younger people, with an average age at diagnosis of 14, and secrete both alpha-fetoprotein (in 75% of cases) and hCG.

Polyembryomas, the most immature form of teratoma and very rare ovarian tumors, are histologically characterized by having several embryo-like bodies with structures resembling a germ disk, yolk sac, and amniotic sac. Syncytiotrophoblast giant cells also occur in poly embry omas.

Primary ovarian squamous cell carcinomas are rare and have a poor prognosis when advanced. More typically, ovarian squamous cell carcinomas are cervical metastases, areas of differentiation in an endometrioid tumor, or derived from a mature teratoma.

Mixed tumors contain elements of more than one of the above classes of tumor histology. To be classed as a mixed tumor, the minor type must make up more than 10% of the tumor. Though mixed carcinomas can have any combination of cell types, mixed ovarian cancers are typically serous/endometrioid or clear cell/endometrioid. Mixed germ cell tumors make up approximately 25-30% of all germ cell ovarian cancers, with combinations of dysgerminoma, yolk sac tumor, and/or immature teratoma.

Ovarian cancer can also be a secondary cancer, the result of metastasis from a primary cancer elsewhere in the body. About 7% of ovarian cancers are due to metastases, while the rest are primary cancers. Common primary cancers are breast cancer, colon cancer, appendiceal cancer, and stomach cancer (primary gastric cancers that metastasize to the ovary are called Krukenberg tumors). Krukenberg tumors have signet ring cells and mucinous cells. Endometrial cancer and lymphomas can also metastasize to the ovary.

It should be appreciated that the methods, compositions and kits of the invention may be applicable for the diagnosis of primary, as well as secondary ovarian carcinoma as discussed herein. Low malignant potential (LMP) ovarian tumors, also called borderline tumors, have some benign and some malignant features. LMP tumors make up approximately 10%-15% of all ovarian tumors. They develop earlier than epithelial ovarian cancer, around the age of 40-49. They typically do not have extensive invasion; 10% of LMP tumors have areas of stromal microinvasion (<3mm, <5% of tumor). LMP tumors have other abnormal features, including increased mitosis, changes in cell size or nucleus size, abnormal nuclei, cell stratification, and small projections on cells (papillary projections). Serous and/or mucinous characteristics can be seen on histological examination, and serous histology makes up the overwhelming majority of advanced LMP tumors. More than 80% of LMP tumors are Stage I; 15% are stage II and III and less than 5% are stage IV. Implants of LMP tumors are often non-invasive.

Ovarian cancer is staged using the FIGO staging system or using the AJCC/TNM staging system.

FIGO stages of ovarian cancer are as follows: at stage I, cancer is completely limited to the ovary. At stage IA, it involves one ovary, the capsule is intact, there is no tumor on ovarian surface, washings are negative. At stage IB, cancer involves both ovaries; the capsule is intact, there is no tumor on ovarian surface, washings are negative. At stage IC, tumor involves one or both ovaries. At stage IC1, there is surgical spill. At stage IC2, the capsule has ruptured or tumor are on ovarian surface. At stage IC3, there are positive ascites or washings. A stage II, one can observe pelvic extension of the tumor (must be confined to the pelvis) or primary peritoneal tumor, it involves one or both ovaries. At stage IIA, tumor is found on uterus or fallopian tubes. At stage IIB, tumor appears elsewhere in the pelvis. At stage III, cancer is found outside the pelvis or in the retroperitoneal lymph nodes, it involves one or both ovaries. At stage IIIA, metastasis appear in retroperitoneal lymph nodes or microscopic extrapelvic metastasis. At stage IIIA1, metastasis is in retroperitoneal lymph nodes. At stage IIIA1(i) the metastasis is less than 10 mm in diameter, at stage IIIA1(ii) the metastasis is greater than 10 mm in diameter. At stage IIIA2, there is microscopic metastasis in the peritoneum, regardless of retroperitoneal lymph node status. At stage IIIB, metastasis appears in the peritoneum less than or equal to 2 cm in diameter, regardless of retroperitoneal lymph node status; or metastasis to liver or spleen capsule. At stage IIIC, metastasis appears in the peritoneum greater than 2 cm in diameter, regardless of retroperitoneal lymph node status; or metastasis to liver or spleen capsule. At stage IV, distant metastasis can be observed (i.e. outside of the peritoneum). At stage IVA, one can observe pleural effusion containing cancer cells. At stage IVB, there is metastasis to distant organs (including the parenchyma of the spleen or liver), or metastasis to the inguinal and extra-abdominal lymph nodes.

The AJCC/TNM staging system indicates where the tumor has developed, spread to lymph nodes, and metastasis AJCC/TNM stages of ovarian cancer are as following: at stage T, primary tumor can be observed. At stage T1, the tumor is limited to ovary/ovaries. At stage T1 a, one ovary has intact capsule, no surface tumor, and ascites/peritoneal washings are negative. At stage T1b, both ovaries have intact capsules, no surface tumor, and ascites/peritoneal washings are negative. At stage T1c, one or both ovaries has ruptured capsule or capsules, surface tumor, ascites/peritoneal washings are positive. At stage T2, tumor is in ovaries and pelvis (extension or implantation). At stage T2a, there is expansion to the uterus or the Fallopian tubes, ascites/peritoneal washings are negative. At stage T2b, there is expansion in other pelvic tissues, ascites/peritoneal washings are negative. At stage T2c, there is expansion to any pelvic tissue, ascites/peritoneal washings are positive. At stage T3, the tumor is in ovaries and has metastasized outside the pelvis to the peritoneum (including the liver capsule). At stage T3a, microscopic metastasis is observed. At stage T3b, macroscopic metastasis is less than 2 cm diameter. At stage T3c, macroscopic metastasis is greater than 2 cm diameter. At stage N, regional lymph node metastasis is observed. At stage N1, metastasis is present. At stage M, there is distant metastasis. At stage M0, no metastasis is observed. At stage M1, metastasis is present (excluding liver capsule, including liver parenchyma and cytologically confirmed pleural effusion).

In addition to being staged, like all cancers, ovarian cancer is also graded. The histologic grade of a tumor measures how abnormal or malignant its cells look under the microscope. The four grades indicate the likelihood of the cancer to spread and the higher the grade, the more likely for this to occur. Grade 0 is used to describe noninvasive tumors. Grade 0 cancers are also referred to as borderline tumors. Grade 1 tumors have well differentiated cells (look very similar to the normal tissue) and are the ones with the best prognosis. Grade 2 tumors are also called moderately well-differentiated and they are made up of cells that resemble the normal tissue. Grade 3 tumors have the worst prognosis and their cells are abnormal, referred to as poorly differentiated.

It should be appreciated that the methods, compositions and kits of the invention may be applicable for the diagnosis or ovarian carcinoma of any of the subgroups, grades, types or stages disclosed herein.

As described in Example 3, the inventors have analyzed the proteomic profiles (Mass spectrometry) of the 9 biomarker proteins both in HGOC patients and control group; they observed that 9 biomarkers were differently expressed in HGOC patients in comparison with the control group. This result was further validated by analysis of gene expression (RT-PCR) of these 9 biomarker proteins as detailed in Example 4.

Thus, in accordance with some embodiments, in the first step (a) of the method of the invention, the expression level of at least one of the biomarker proteins described herein is being determined. The terms “level of expression” or “expression level” are used interchangeably and generally refer to a numerical representation of the amount (quantity) of an amino acid product or polypeptide or protein in a biological sample. In yet some further embodiments, the “level of expression” or “expression level” refers to the numerical representation of the amount (quantity) of polynucleotide which may be gene in a biological sample.

“Expression” generally refers to the process by which gene-encoded information is converted into the structures present and operating in the cell. For example, gene expression values may be measured in the protein level, for example by MS methods or alternatively by immunological methods. Alternatively, the expression may be measured in the nucleic acid level, for example using Real-Time Polymerase Chain Reaction, sometimes also referred to as RT-PCR or quantitative PCR (qPCR). The luminosity in case of RT-PCR, or any other tag is captured by a detector that converts the signal intensity into a numerical representation which is said expression value, in terms of biomarker protein or a gene. Therefore, according to the invention “expression” of a gene, specifically, any gene encoding any of the biomarker proteins of the invention may refer to transcription into a polynucleotide and translation into a polypeptide. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. Methods for determining the level of expression of the biomarkers of the invention will be described in more detail herein after. It should be appreciated that the methods of the invention, as well as the compositions and kits disclosed herein after, refer to the level of the biomarker protein/s in the sample. It should be understood that the level of the protein reflects the level of expression but may also reflect the stability of the biomarker protein.

The expression level of the biomarker proteins of the invention is determined to obtain an expression value. The term “expression value” refers to the result of a calculation, that uses as an input the “level of expression” or “expression level” obtained experimentally. It should be appreciated that in some optional embodiments, determination of the expression value may further involves normalizing the “level of expression” or “expression level” by at least one normalization step as detailed herein, where the resulting calculated value termed herein “expression value” is obtained.

More specifically, as used herein, “normalized values” in some embodiments, are the quotient of raw expression values of marker proteins, divided by the expression value of a control reference protein from the same sample. Any assayed sample may contain more or less biological material than is intended, due to human error and equipment failures. Importantly, the same error or deviation applies to both the marker protein of the invention and to the control reference protein, whose expression is essentially constant. Thus, division of the marker protein raw expression value by the control reference protein raw expression value yields a quotient which is essentially free from any technical failures or inaccuracies (except for major errors which destroy the sample for testing purposes) and constitutes a normalized expression value of said marker protein. This normalized expression value may then be compared with normalized cutoff values, i.e., cutoff values calculated from normalized expression values. In certain embodiments, the control reference protein may be a protein that maintains stable in all samples analyzed. Normalized biomarker protein expression level values that are higher (positive) or lower (negative) in comparison with a corresponding predetermined standard expression value or a cut-off value in a control sample predict to which population of subjects, either healthy or diseased, the tested sample belongs, and in some embodiments, may even reflect the disease stage, or the metastatic status of the subject.

It should be appreciated that an important step in the method of the inventions is determining whether the expression value of any one of the biomarker proteins is changed or different when compared to a pre-determined cut off, or is within the range of expression of such cutoff. Alternatively, or in addition, the expression value may be compared to the expression value of a control sample, for example, a sample obtained from a healthy subject or from a subject that is not affected by ovarian cancer.

Thus, in yet more specific embodiments, the second step (b) of the method of the invention involves comparing the expression values determined for the tested sample with predetermined standard values or cutoff values, or alternatively, with expression values of at least one control sample. As used herein the term “comparing” denotes any examination of the expression level and/or expression values obtained in the samples of the invention as detailed throughout in order to discover similarities or differences between at least two different samples. It should be noted that in some embodiments, comparing according to the present invention encompasses the possibility to use a computer based approach.

As described hereinabove, the method of the invention refers to a predetermined cutoff value/s. It should be noted that a “cutoff value”, sometimes referred to simply as “cutoff” herein, is a value that meets the requirements for both high diagnostic sensitivity (true positive rate) and high diagnostic specificity (true negative rate).

It should be noted that the terms “sensitivity” and “specificity” are used herein with respect to the ability of one or more markers, to correctly classify a sample as belonging to a pre-established population associated with ovarian cancer, specifically, HGOC (or type II), or alternatively, to a pre-established population of healthy subjects or subjects that are not affected by HGOC. In other words, to correctly classify a sample as a sample of a subject affected by ovarian cancer or alternatively as a subject that is not affected by ovarian cancer (either healthy or not).

“Sensitivity” indicates the performance of the biomarker of the invention, with respect to correctly classifying samples as belonging to pre-established populations that are likely to suffer from a disease or disorder or characterized at different stages of a disease, wherein said biomarker are consider here as any of the options provided herein.

“Specificity” indicates the performance of the biomarker of the invention with respect to correctly classifying and distinguishing between samples as belonging to pre-established populations of subjects suffering from the same disorder and populations of subjects that are either healthy or not affected by ovarian cancer.

Simply put, “sensitivity” relates to the rate of identification of the patients (samples) as such out of a group of samples, whereas “specificity” relates to the rate of correct identification of ovarian cancer samples as such out of a group of samples. Cutoff values may be used as control sample/s or in addition to control sample/s, said cutoff values being the result of a statistical analysis of biomarker protein expression value/s (specifically the biomarker/s proteins of the invention) differences in pre-established populations healthy or suffering from ovarian cancer, more specifically suffering from high-grade ovarian carcinoma. Pre-established populations as used herein refer to populations of patients diagnosed with ovarian cancer (by any conventional means), or alternatively, populations of healthy subjects.

In yet some further embodiments, a negative or positive determination of the expression value as compared to the predetermined cutoff values, or the expression value of a control sample, also encompass values that are within the range of said cutoff. More specifically, in case the particular biomarker is found to be overexpressed in ovarian cancer, an expression value that is determined by the method of the invention as “positive” when compared to a predetermined cutoff of population of patients suffering from ovarian cancer, or to the expression value of at least one, and preferably, more, known patient/s suffering from ovarian cancer, may indicate that the examined subject belongs to a population suffering from ovarian cancer (e.g., that the subject carries or is affected by ovarian cancer), in case that the expression value is either higher (positive) or fall within the range (the average values of the cutoff predetermined for patient population suffering from ovarian cancer) of the control or standard value. In a similar manner, a subject exhibiting an expression value that is “negative” (that is down-regulated) as compared to the cutoff patients, may be considered as belonging to population that is not suffering from ovarian cancer, in case the expression of the particular biomarker is associated with overexpression in ovarian cancer. In more specific embodiments, the expression value of such subject should fall within the range of the cutoff value predetermined for population that is not suffering from ovarian cancer. In some embodiments, “fall within the range” encompass values that differ from the cutoff value in about 1%, about 5%, about 10%, about 15%, about 20%, about 25%, about 30%, about 35%, about 40%, about 45%, about 50% or more. Simply put, a “positive” expression value as used herein refers to high expression value that reflects overexpression, elevated expression, high expression and even in some embodiments, moderate expression value. A “negative” expression value reflects a repressed, low, reduced, or non-existing expression (lack of expression). Thus, in some embodiments, when a specific biomarker is overexpressed in ovarian cancer, a “positive” expression value of an examined sample may be a value that is higher or within the range of the expression value of a sample taken from a patient affected with ovarian cancer, or a standard cutoff value calculated for ovarian cancer patients. A “negative” value would be an expression value that is lower than the expression value of the ovarian cancer patients (or standard value, or the value of a control sample). Such value may be within the range of the value of a healthy control sample or a standard value of a healthy population of subject, or of subjects that are not affected by ovarian cancer. In yet some further embodiments, when the specific biomarker is associated with low expression or even non-expression (undetectable expression) in ovarian cancer, a “positive” expression value reflects a value that is higher than the value of the ovarian cancer control or standard value. Such value is not within the range of the value of the ovarian cancer population or control sample, but may be within the range of the value of the “healthy controls” (as used herein, “healthy controls” may include any subject not affected by ovarian cancer). A “negative” value is meant an expression value that is lower than the expression value of the healthy control that is in that case, within the range of the expression value of ovarian cancer patients.

It should be appreciated that a “control sample” as used herein may reflect a sample of at least one subject (either healthy, a subject that is not affected by ovarian cancer, or alternatively, an ovarian cancer patient), and preferably, a mixture at least two, at least three, at least four, at least five, at least six or more patients.

It should be emphasized that the nature of the invention is such that the accumulation of further patient data may improve the accuracy of the presently provided cutoff values, which are based on an ROC (Receiver Operating Characteristic) curve generated according to said patient data using analytical software program. The biomarker protein expression values are selected along the ROC curve for optimal combination of diagnostic sensitivity and diagnostic specificity which are as close to 100 percent as possible, and the resulting values are used as the cutoff values that distinguish between subjects who are diagnosed with positive HGOC at a certain rate, and those who will not (with said given sensitivity and specificity). Similar analysis may be performed for example when diagnosis of cancer is being examined to distingue between healthy tissue and cancerous tissue. The ROC curve may evolve as more and more data and related biomarker gene expression values are recorded and taken into consideration, modifying the optimal cutoff values and improving sensitivity and specificity. Thus, it should be appreciated that the provided cutoff values should be viewed as a starting point that may shift as more data allows more accurate cutoff value calculation. Although considered as initial cutoff values, the presently provided values already provide good sensitivity and specificity, and are readily applicable in current clinical use, even in patients diagnosed with different cancer stages.

As noted above, the expression value determined for the examined sample (or alternatively, the normalized expression value) is compared with a predetermined cutoff or to a control sample. More specifically, in certain embodiments, the expression value obtained for the examined sample is compared with a predetermined standard or cutoff value.

In further embodiments, the predetermined standard expression value, or cutoff value has been pre-determined and calculated for a population comprising at least one of healthy subjects, subjects suffering from any disorder, subjects suffering from different stages of any disorder, subjects that respond to treatment, non-responder subjects, subjects in remission and subjects in relapse.

Still further, in certain alternative embodiments where a control sample is being used (instead of, or in addition to, pre-determined cutoff values), the expression value or the normalized expression values of the biomarker proteins used by the invention in the test sample are compared to the expression values in the control sample. In certain embodiments, such control sample may be obtained from at least one of a healthy subject, a subject suffering from a disorder at a specific stage, a subject suffering from a disorder at a different specific stage a subject that responds to treatment, a non-responder subject, a subject in remission and a subject in relapse

It should be appreciated that “Standard” or a “predetermined standard” as used herein, denotes either a single standard value or a plurality of standards with which the level of at least one of the biomarker protein expression from the tested sample is compared. The standards may be provided, for example, in the form of discrete numeric values or is calorimetric in the form of a chart with different colors or shadings for different levels of expression; or they may be provided in the form of a comparative curve prepared on the basis of such standards (standard curve).

It should be noted that for determining the expression value/s of at least one of the biomarker proteins of the invention, the methods of the invention may further comprise the step of providing at least one detecting molecule specific for determining the expression of at least on of said biomarker proteins of the invention. In some embodiments, such detecting molecules may be provided as a mixture, as a composition or as a kit. Thus, in some embodiments, the at least one detecting molecules may be provided as a mixture of detecting molecules, wherein each detecting molecule is specific for one biomarker protein. It should be appreciated however, that for each biomarker protein, one or several specific detecting molecules may be used and provided. In yet some further alternative embodiments, the detecting molecules may be provided separately for each biomarker protein, e.g., in specific tube, containers, slots, spots, wells, and the like. It further alternative embodiments, the detecting molecules may be attached or immobilized to a solid support, specifically, in recorded location.

Still further, it should be noted that all steps for determining the different parameters indicated above, involve contacting the sample or any component thereof with a specific reagent (e.g., detecting molecules).

Thus, in yet some specific embodiments, the method of the invention may involves determining the level of expression of at least one, of at least two, at least three, at least four, at least five, at least six, at least seven, at least eight or at least nine of said CLCA4, OVGP1, S100A14, SPRR3, RNASE3, SERPINB5, CLUAP1, CEACAM5 and ENPP3 biomarker protein/s, by performing the step of contacting at least one detecting molecule or any combination or mixture of plurality of detecting molecules with a biological sample of said subject, or with any protein or nucleic acid product obtained therefrom. It should be noted that each of said detecting molecules is specific for one of said biomarker proteins.

The term “contacting” mean to bring, put, incubates or mix together. As such, a first item is contacted with a second item when the two items are brought or put together, e.g., by touching them to each other or combining them. In the context of the present invention, the term “contacting” includes all measures or steps which allow interaction between the at least one of the detection molecules of at least one of the biomarker proteins, and optionally, for at least one suitable control reference protein of the tested sample. The contacting is performed in a manner so that the at least one of detecting molecule of at least one of the biomarker proteins for example, can interact with or bind to the at least one of the biomarker proteins, in the tested sample. The binding will preferably be non-covalent, reversible binding, e.g., binding via salt bridges, hydrogen bonds, hydrophobic interactions or a combination thereof.

In certain embodiments, the detection step further involves detecting a signal from the detecting molecules that correlates with the expression level of at least one of the biomarker proteins in the sample from the subject, by a suitable means. According to some embodiments, the signal detected from the sample by any one of the experimental methods detailed herein below reflects the expression level of at least one of the biomarker proteins. It should be noted that such signal-to-expression level data may be calculated and derived from a calibration curve.

Thus, in certain embodiments, the method of the invention may optionally further involve the use of a calibration curve created by detecting a signal for each one of increasing pre-determined concentrations of at least one of the biomarker proteins. Obtaining such a calibration curve may be indicative to evaluate the range at which the expression levels correlate linearly with the concentrations of at least one of the biomarker proteins. It should be noted in this connection that at times when no change in expression level of at least one of the biomarker proteins is observed, the calibration curve should be evaluated in order to rule out the possibility that the measured expression level is not exhibiting a saturation type curve, namely a range at which increasing concentrations exhibit the same signal.

It must be appreciated that in certain embodiments such calibration curve as described above may be also part or component in any of the kits provided by the invention as described herein after.

In other embodiments of the invention, the detecting molecules used for determining the expression levels at least one of the biomarker proteins may be selected from isolated detecting amino acid molecules and isolated detecting nucleic acid molecules. It should be noted that the invention further encompasses any combination of nucleic and amino acids for use as detecting molecules for the methods of the invention. As noted above, in the first step of the method of the invention, the sample or any protein or nucleic acid obtained therefrom, is contacted with the detecting molecules of the invention.

The invention thus contemplates the use of amino acid based molecules such as proteins or polypeptides as detecting molecules disclosed herein and would be known by a person skilled in the art to measure the at least one biomarker protein. As used herein, the terms “protein” and “polypeptide” are used interchangeably to refer to a chain of amino acids linked together by peptide bonds. In a specific embodiment, a protein is composed of less than 200, less than 175, less than 150, less than 125, less than 100, less than 50, less than 45, less than 40, less than 35, less than 30, less than 25, less than 20, less than 15, less than 10, or less than 5 amino acids linked together by peptide bonds. In another embodiment, a protein is composed of at least 200, at least 250, at least 300, at least 350, at least 400, at least 450, at least 500, at least 1000 or more amino acids linked together by peptide bonds. It should be noted that peptide bond as described herein is a covalent amid bond formed between two amino acid residues. In some embodiments, the detecting molecules used by the methods of the invention may be recombinantly expressed or synthetically prepared. In further embodiments, the recombinantly or synthetically expressed and prepared detecting molecules may be labeled or tagged. It should be noted that in some embodiments, these detecting molecules may be isolated detecting molecules. As used herein, “Recombinant proteins” denotes proteins encoded by a recombinant DNA which is a genetically engineered DNA formed by laboratory methods of genetic recombination to bring together genetic material from multiple sources and thus creating variable sequences. Recombinant proteins may be produced mainly, but not limited, by molecular cloning, namely incorporating the recombinant DNA into a living cell (e.g. bacteria or yeast) and using its system to express the DNA into mRNA and protein thereof.

Techniques for detection and quantification known to persons skilled in the art (for example, Mass spectrometry (MS) or different immunological techniques such as Western Blotting, Immunoprecipitation, ELISAs, protein microarray analysis, Flow cytometry and the like) can then be used to measure the level of protein products corresponding to the biomarker of the invention.

In certain embodiments, the amino acid detecting molecule/s suitable for the method of the invention may comprise at least one of: (a) at least one labeled or tagged CLCA4, OVGP1, S100A14, SPRR3, RNASE3, SERPINB5, CLUAP1, CEACAM5 and ENPP3 protein/s or any fragment/s, peptide/s or mixture/s thereof; (b) at least one antibody specific for said at least one of said biomarker proteins; (c) at least one peptide aptamer/s specific for said at least one of said biomarker proteins; and (d) any combination of (a), (b) and (c).

More specifically, in some embodiments, the detecting molecules may be at least one labeled or tagged CLCA4, OVGP1, S100A14, SPRR3, RNASE3, SERPINB5, CLUAP1, CEACAM5 and ENPP3protein/s or any fragments, peptides or mixture thereof.

Still further, in certain alternative or additional embodiments, the amino acid detecting molecule/s suitable for the method of the invention may comprise in addition to the at least one of the 9-signatory biomarkers of the invention, at least one of: (a) at least one labeled or tagged C1RL, AGRN, ADIRF, ITLN1, CPM, VCAN, BCAT1, NDRG3, CD109, CDH1, ADH1B, GULP1, GLUL, CD34, CRNN, SERPINB5, CEACAM6, LGALS7, S100A14, THY1 and GLRX3 protein/s or any fragment/s, peptide/s or mixture/s thereof; (b) at least one antibody specific for said at least one of said biomarker proteins; (c) at least one peptide aptamer/s specific for said at least one of said biomarker proteins; and (d) any combination of (a), (b) and (c).

In some embodiments, the term “labeled” or “tagged” may refer to direct labeling of the protein via, e.g., coupling (i.e., physically linking) or incorporating of a detectable substance to the protein. Useful labels in the present invention may include but are not limited to include isotopes (e.g. ¹³C, ¹⁵N), or any other radiolabels (e.g., ³H, ¹²⁵I, ³⁵S, ¹⁴C, or ³²P), magnetic beads (e.g. DYNABEADS), fluorescent dyes (e.g., fluorescein isothiocyanate, Texas red, rhodamine, green fluorescent protein, and the like), enzymes (e.g., horseradish peroxidase, alkaline phosphatase and others commonly used in an ELISA and competitive ELISA, histochemistry and other similar methods known in the art) and colorimetric labels such as colloidal gold or colored glass or plastic (e.g. polystyrene, polypropylene, latex, etc.) beads. In some embodiments, the protein may be tagged. Different tags may be also used, for example, His, myc, HA, GFP, ABP, GST, biotin and the like. “tagged” as used herein may further include fusion or linking of the biomarker protein or any fragment or peptide thereof, that serves herein as a detecting molecule, a tag that in some embodiments may contain several amino acids or a peptide that may be recognized by affinity or immunologically, using specific antibodies.

In some other embodiments, the biomarker proteins or any fragments or peptides thereof may be fluorescently labeled. In another embodiment, the biomarker proteins or any fragments or peptides thereof may be isotope labeled. The term “recombinant isotope labeled” denotes a protein ‘labeled’ by replacing specific atoms by their isotope.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

More specifically, in certain embodiments the biomarker proteins of the invention or any fragment or peptide thereof, when recombinantly expressed and labeled or tagged, may be used as detecting molecules for determining the quantity or level of expression of the biomarker proteins of the invention in the examined sample. The term “labeled form” as used herein includes an isotope labeled form. Specifically, the labeled form is a chemically or metabolically isotope labeled, and more specifically a metabolically isotope labeled form of the biomarker proteins of the invention.

Optional “isotope labeled forms” of the biomarker protein/s or any fragments or peptides thereof in accordance with the present invention are variants of naturally occurring molecules, in whose structure one or more atoms have been substituted with atom(s) of the same element having a different atomic weight, although isotope labeled forms in which the isotope has been covalently linked either directly or via a linker, or wherein the isotope has been complexed to the biomarker proteins are likewise contemplated. In either case, the isotope may be stable isotope. A stable isotope as referred to herein, is a non-radioactive isotopic form of an element having identical numbers of protons and electrons, but having one or more additional neutron(s), which increase(s) the molecular weight of the element. Specifically, the stable isotopes may be selected from the group consisting of ²_{H,} ¹³_{C,} ¹⁵N, ¹⁷0, ¹⁸0, ³³P, ³⁴S and combinations thereof. Particularly specific examples include ¹³C and ⁵N, and combinations thereof.

The labeling can be effected by means known in the art. A labeled reference biomarker (used as detecting molecule) can be synthesized using isotope labeled amino acids as precursor molecules, or chemically modified. Modification and labeling can be done on whole proteins or their fragments. For example, isotope-coded affinity tag (ICAT) reagents label reference biomolecule such as proteins at the alkylation step of sample preparation (WO2004079370). Visible ICAT reagents (VICAT reagents) may be likewise employed (WO2011042467), whereby the VICAT-type reagent contains as a detectable moiety a fluorophore or radiolabel. iTRAQ and similar methods may likewise be employed.

Metabolic labeling may also be used to produce the labeled reference biomarkers. For example, cells can be grown on media containing isotope labeled precursor molecules, such as isotope labeled amino acids, that are incorporated into proteins or peptides, which are thereby metabolically labeled. The metabolic isotope labeling may be a stable isotope labeling with amino acids in cell culture (SILAC). If metabolic labeling is used, and the labeled form of the one or the plurality of reference biomarker protein/s is a SILAC labeled form of the reference biomarker protein/s, the standard mixture as defined above is also referred to as SUPER-SILAC mix.

In specific embodiments, the detecting amino acid molecules applicable for the invention may be isolated antibodies, with specific binding selectively to at least one of said biomarker proteins. More specifically, antibodies that specifically bind at least one of the biomarker proteins of the invention as listed in Table 4, specifically, at least one of CLCA4, OVGP1, S100A14, SPRR3, RNASE3, SERPINB5, CLUAP1, CEACAM5 and ENPP3, and optionally, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1, GLRX3, PAFAH1B2, GPC4, CKB, BPI, GSTT1, SET, ENPP1, MPDZ, ALDH1L1, IGFBP4 and SFRP1. It should be understood that each antibody specifically recognizes one biomarker protein. Using these antibodies, the level of expression of at least one of the biomarker protein may be determined using an immunoassay which may be an assay that includes but not limited to FACS, a Western blot, an ELISA, a RIA, a slot blot, a dot blot, immune-histochemical assay and a radio-imaging assay. It should be noted that such assay may be performed using microarray protein arrays.

More specifically, he term “antibody” as used in this invention includes whole antibody molecules as well as functional fragments thereof, such as Fab, F(ab′)2, and Fv that are capable of binding with antigenic portions of the target polypeptide, i.e. at least one of the biomarker protein. The antibody may be preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition”, which, as used herein, refer to a preparation of antibodies or fragments thereof of single molecular composition.

It should be recognized that the antibody can be a human antibody, a chimeric antibody, a recombinant antibody, a humanized antibody, a monoclonal antibody, or a polyclonal antibody. The antibody can be an intact immuno globulin, e.g., an IgA, IgG, IgE, IgD, 1gM or subtypes thereof. The antibody can be conjugated to a labeling moiety as discussed above. As noted above, the term “antibody” also encompasses antigen-binding fragments of an antibody. The term “antigen-binding fragment” of an antibody (or simply “antibody portion,” or “fragment”), as used herein, may be defined as follows:

(1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab′)2 is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”, or ScFv), a genetically engineered molecule containing the variable region of the light chain and the variable region of the heavy chain, linked by a suitable polypeptide linker as a genetically fused single chain molecule.

Methods of generating such antibody fragments are well known in the art (See for example, Harlow and Lane, Antibodies: A Laboratory Manual, Cold Spring Harbor Laboratory, New York, 1988, incorporated herein by reference).

Purification of serum immunoglobulin antibodies (polyclonal antisera) or reactive portions thereof can be accomplished by a variety of methods known to those of skill in the art including, precipitation by ammonium sulfate or sodium sulfate followed by dialysis against saline, ion exchange chromatography, affinity or immuno-affinity chromatography as well as gel filtration, zone electrophoresis, etc.

Still further, the antibodies used by the present invention may optionally be covalently or non-covalently linked to a detectable label or tag. In addition, the label and can also refer to indirect labeling of the protein by reactivity with another reagent that is directly labeled. Examples of indirect labeling include detection of at least one of the biomarker protein/s of the invention using a fluorescently labeled secondary antibody. More specifically, detectable labels suitable for such use include any composition detectable by spectroscopic, photochemical, biochemical, immunochemical, electrical, optical or chemical means.

The antibody used as a detecting molecule according to the invention, specifically recognizes and binds at least one of the biomarker protein. It should be noted that in certain embodiments, each antibody is specific for one of the biomarker proteins of the invention, specifically, those disclosed in Table 4, specifically, at least one of CLCA4, OVGP1, S100A14, SPRR3, RNASE3, SERPINB5, CLUAP1, CEACAM5 and ENPP3, and optionally, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1, GLRX3, PAFAH1B2, GPC4, CKB, BPI, GSTT1, SET, ENPP1, MPDZ, ALDH1L1, IGFBP4 and SFRP1 or any further marker protein. It should be appreciated that antibodies that may be used by the methods as well as the compositions and kits of the invention, may be antibodies directed not only against the biomarker proteins of the invention, but also in case the biomarkers are tagged, the antibodies may be directed against said tags. It should be therefore noted that the term “binding specificity”, “specifically binds to an antigen”, “specifically immuno-reactive with”, “specifically directed against” or “specifically recognizes”, when referring to an epitope, specifically, a recognized epitope within the at least one of the biomarker protein, refers to a binding reaction which is determinative of the presence of the epitope in a heterogeneous population of proteins and other biologics. More particularly, “selectively bind” in the context of proteins encompassed by the invention refers to the specific interaction of any two of a peptide, a protein, a polypeptide an antibody, wherein the interaction preferentially occurs as between any two of a peptide, protein, polypeptide and antibody preferentially as compared with any other peptide, protein, polypeptide and antibody.

Thus, under designated immunoassay conditions, the specified antibodies bind to a particular epitope at least two times the background and more typically more than 10 to 100 times background. More specifically, “Selective binding”, as the term is used herein, means that a molecule binds its specific binding partner with at least 2-fold greater affinity, and preferably at least 10-fold, 20-fold, 50-fold, 100-fold or higher affinity than it binds a non-specific molecule. It should be appreciated that the antibodies used by the methods of the invention, may be in some embodiments antibodies that are not naturally occurring antibodies. More specifically, the antibodies are not produced naturally in the body, and more specifically, it should be appreciated that production thereof involves immunological and recombinant techniques.

A variety of immunoassay formats may be used to select antibodies specifically immuno-reactive with a particular protein or carbohydrate. For example, solid-phase ELISA immunoassays are routinely used to select antibodies specifically immuno-reactive with a protein or carbohydrate. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

In some other embodiments, the detecting molecules are peptide aptamers specific for said at least one of said biomarker proteins. “Peptide or protein aptamers” as used herein refers to small peptides with a single variable loop region tied to a protein scaffold on both ends that binds to a specific molecular target (e.g. protein), and which are bind to their targets only with said variable loop region and usually with high specificity properties.

According to one embodiment, where amino acid-based detection molecules are used, the expression level of the at least one of the biomarker protein, in the tested sample can be determined using different methods known in the art, specifically method disclosed herein below as non-limiting examples.

In some alternative embodiments, determination of the expression levels of the biomarker proteins of the invention may be performed in the nucleic acid level, specifically, the mRNA level. In such embodiments for determining the expression level of the biomarkers of the invention, nucleic acid detecting molecule may be used.

In some embodiments, the nucleic acid detecting molecule/s of the invention may comprise at least one of: (a) nucleic acid aptamers specific for said at least one of said biomarker proteins; and (b) at least one isolated oligonucleotides, each oligonucleotide specifically hybridizes to a nucleic acid sequence encoding said at least one biomarker protein.

As used herein, “nucleic acid molecules” or “nucleic acid sequence” are interchangeable with the term “polynucleotide(s)” and it generally refers to any polyribonucleotide or poly-deoxyribonucleotide, which may be unmodified RNA or DNA or modified RNA or DNA or any combination thereof “Nucleic acids” include, without limitation, single- and double-stranded nucleic acids. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also include regions of single- or double- stranded RNA or DNA or any combinations.

More specifically, in some other embodiments, the nucleic acid detecting molecules may comprise at least one isolated oligonucleotide/s, each oligonucleotide specifically hybridizes to a nucleic acid sequence encoding one of said at least one biomarker protein. In an optional embodiment, where the expression levels of the biomarkers of the invention are normalized, the method of the invention may use nucleic acid detecting molecules specific for a nucleic acid sequence encoding the control reference protein/s.

As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from about 3 to about 1,000 nucleotides long. Although oligonucleotides of 5 to 100 nucleotides are useful in the invention, preferred oligonucleotides range from about 5 to about 15 bases in length, from about 5 to about 20 bases in length, from about 5 to about 25 bases in length, from about 5 to about 30 bases in length, from about 5 to about 40 bases in length or from about 5 to about 50 bases in length. More specifically, the detecting oligonucleotides molecule used by the composition of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 bases in length. It should be further noted that the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly. In yet some further specific embodiments, where the detecting molecules of the invention are nucleic acid based molecules, optional detecting molecule/s may be at least one nucleic acid aptamer specific for the at least one of said biomarker proteins.

As used herein the term “aptamer” or “specific aptamers” denotes single-stranded nucleic acid (DNA or RNA) molecules which specifically recognizes and binds to a target molecule. The aptamers according to the invention may fold into a defined tertiary structure and can bind a specific target molecule with high specificities and affinities. Aptamers are usually obtained by selection from a large random sequence library, using methods well known in the art, such as SELEX and/or Molinex. In various embodiments, aptamers may include single-stranded, partially single-stranded, partially double-stranded or double-stranded nucleic acid sequences; sequences comprising nucleotides, ribonucleotides, deoxyribonucleotides, nucleotide analogs, modified nucleotides and nucleotides comprising backbone modifications, branch points and non-nucleotide residues, groups or bridges; synthetic RNA, DNA and chimeric nucleotides, hybrids, duplexes, heteroduplexes; and any ribonucleotide, deoxyribonucleotide or chimeric counterpart thereof and/or corresponding complementary sequence. In certain specific embodiments, aptamers used by the invention are composed of deoxyribonucleotides. According to the present invention and as appreciated in the art, the recognition between the aptamer and the antigen is specific and may be detected by the appearance of a detectable signal by using a colorimetric sensor or a fluorimetric/lumination sensor, radioactive sensor, or any appropriate means.

The aptamers that may be used according to some aspects of the invention may be biotinylated. The aptamers may optionally include a chemically reactive group at the 3′ and/or 5′ termini. The term reactive group is used herein to denote any functional group comprising a group of atoms which is found in a molecule and is involved in chemical reactions. Some non-limiting examples for a reactive group include primary amines (NH₂), thiol (SH), carboxy group (COOH), phosphates (PO4), Tosyl, and a photo-reactive group.

In some embodiments, the aptamer that may be applicable herein may optionally comprise a spacer between the nucleic acid sequence and the reactive group. The spacer may be an alkyl chain such as (CH₂)_{6/12,} namely comprising six to twelve carbon atoms. In yet some other alternative embodiments, the detection molecule may be at least one primer, at least one pair of primers, nucleotide probes and any combinations thereof Thus, it should be further appreciated that the methods, as well as the compositions and kits of the invention may comprise, as an oligonucleotide-based detection molecule, both primers and probes.

The term, “primer”, as used herein refers to an oligonucleotide, whether occurring naturally as in a purified restriction digest, or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single- stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 10-30 or more nucleotides, although it may contain fewer nucleotides. More specifically, the primer used by the methods, as well as the compositions and kits of the invention may comprise 10, 11, 12, 13, 14, 15, 16, 17, 18, 19, 20, 21, 22, 23, 24, 25, 26, 27, 28, 29 or 30 nucleotides or more. In certain embodiments, such primers may comprise 30, 40, 50, 60, 70, 80, 90, 100 nucleotides or more. In specific embodiments, the primers used by the method of the invention may have a stem and loop structure. The factors involved in determining the appropriate length of primer are known to one of ordinary skill in the art and information regarding them is readily available. As used herein, the term “probe” means oligonucleotides and analogs thereof and refers to a range of chemical species that recognize polynucleotide target sequences through hydrogen bonding interactions with the nucleotide bases of the target sequences. The probe or the target sequences may be single- or double-stranded RNA or single- or double- stranded DNA or a combination of DNA and RNA bases. A probe may be 5, 6, 7, 8, 9, 10, 11, 12, 13, 14, 15, 16, 17, 18, 19, 20, 21, 22, 23, 24, 25, 26, 27, 28, 29 and up to 30 or more nucleotides in length as long as it is less than the full length of the target mRNA or any gene encoding said mRNA. Probes can include oligonucleotides modified so as to have a tag which is detectable by fluorescence, chemiluminescence and the like. The probe can also be modified so as to have both a detectable tag and a quencher molecule, for example TaqMan(R) and Molecular Beacon(R) probes.

The oligonucleotides and analogs thereof may be RNA or DNA, or analogs of RNA or DNA, commonly referred to as antisense oligomers or antisense oligonucleotides. Such RNA or DNA analogs comprise, but are not limited to, 2-'0-alkyl sugar modifications, methylphosphonate, phosphorothiate, phosphorodithioate, formacetal, 3-thioformacetal, sulfone, sulfamate, and nitroxide backbone modifications, and analogs, for example, LNA analogs, wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs. Probes may also be mixtures of any of the oligonucleotide analog types together or in combination with native DNA or RNA. At the same time, the oligonucleotides and analogs thereof may be used alone or in combination with one or more additional oligonucleotides or analogs thereof.

According to this option, the expression level may be determined using amplification assay. The term “amplification assay”, with respect to nucleic acid sequences, refers to methods that increase the representation of a population of nucleic acid sequences in a sample. Nucleic acid amplification methods, such as PCR, isothermal methods, rolling circle methods, etc., are well known to the skilled artisan. More specifically, as used herein, the term “amplified”, when applied to a nucleic acid sequence, refers to a process whereby one or more copies of a particular nucleic acid sequence is generated from a template nucleic acid, preferably by the method of polymerase chain reaction.

“Polymerase chain reaction” or “PCR” refers to an in vitro method for amplifying a specific nucleic acid template sequence. The PCR reaction involves a repetitive series of temperature cycles and is typically performed in a volume of 50-100 microliter. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers”, “a set of PCR primers” or “pair of primers” can comprise two, three, four or more primers.

Real time nucleic acid amplification and detection methods are efficient for sequence identification and quantification of a target since no pre-hybridization amplification is required. Amplification and hybridization are combined in a single step and can be performed in a fully automated, large-scale, closed-tube format. Example 4 demonstrates the use of a nucleic acid based detection method.

Methods that use hybridization-triggered fluorescent probes for real time PCR are based either on a quench-release fluorescence of a probe digested by DNA Polymerase (e.g., methods using TaqMan(R), MGB-TaqMan(R)), or on a hybridization-triggered fluorescence of intact probes (e.g., molecular beacons, and linear probes). In general, the probes are designed to hybridize to an internal region of a PCR product during annealing stage (also referred to as amplicon). For those methods utilizing TaqMan(R) and MGB-TaqMan(R) the 5′-exonuclease activity of the approaching DNA Polymerase cleaves a probe between a fluorophore and a quencher, releasing fluorescence.

Thus, a “real time PCR” or “RT-PCT” assay provides dynamic fluorescence detection of amplified biomarker proteins of the invention or any control reference gene produced in a PCR amplification reaction. During PCR, the amplified products created using suitable primers hybridize to probe nucleic acids (TaqMan(R) probe, for example), which may be labeled according to some embodiments with both a reporter dye and a quencher dye. When these two dyes are in close proximity, i.e. both are present in an intact probe oligonucleotide, the fluorescence of the reporter dye is suppressed. However, a polymerase, such as AmpliTaq Gold™, having 5′-3′ nuclease activity can be provided in the PCR reaction. This enzyme cleaves the fluorogenic probe if it is bound specifically to the target nucleic acid sequences between the priming sites. The reporter dye and quencher dye are separated upon cleavage, permitting fluorescent detection of the reporter dye. Upon excitation by a laser provided, e.g., by a sequencing apparatus, the fluorescent signal produced by the reporter dye is detected and/or quantified. The increase in fluorescence is a direct consequence of amplification of target nucleic acids during PCR.

More particularly, QRT-PCR or “qPCR” (Quantitative RT-PCR), which is quantitative in nature, can also be performed to provide a quantitative measure of gene expression levels. In QRT-PCR reverse transcription and PCR can be performed in two steps, or reverse transcription combined with PCR can be performed. One of these techniques, for which there are commercially available kits such as TaqMan(R) (Perkin Elmer, Foster City, Calif.), is performed with a transcript-specific antisense probe. This probe is specific for the PCR product (e.g. a nucleic acid fragment derived from a gene) and is prepared with a quencher and fluorescent reporter probe attached to the 5′ end of the oligonucleotide. Different fluorescent markers are attached to different reporters, allowing for measurement of at least two products in one reaction.

When Taq DNA polymerase is activated, it cleaves off the fluorescent reporters of the probe bound to the template by virtue of its 5-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions can be performed in any solid support, for example, slides, microplates, 96 well plates, 384 well plates and the like so that samples derived from many individuals are processed and measured simultaneously. The TaqMan(R) system has the additional advantage of not requiring gel electrophoresis and allows for quantification when used with a standard curve.

A second technique useful for detecting PCR products quantitatively without is to use an intercalating dye such as the commercially available QuantiTect SYBR Green PCR (Qiagen, Valencia Calif.). RT-PCR is performed using SYBR green as a fluorescent label which is incorporated into the PCR product during the PCR stage and produces fluorescence proportional to the amount of PCR product.

Both TaqMan(R) and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other known systems to quantitatively measure mRNA expression products include Molecular Beacons(R) which uses a probe having a fluorescent molecule and a quencher molecule, the probe capable of forming a hairpin structure such that when in the hairpin form, the fluorescence molecule is quenched, and when hybridized, the fluorescence increases giving a quantitative measurement of gene expression.

According to this embodiment, the detecting molecule may be in the form of probe corresponding and thereby hybridizing to any region or at least one of the biomarker protein or any control reference protein. More particularly, it is important to choose regions which will permit hybridization to the target nucleic acids. Factors such as the Tm of the oligonucleotide, the percent GC content, the degree of secondary structure and the length of nucleic acid are important factors.

It should be noted however that a standard Northern blot assay or dot blot can also be used to ascertain an RNA transcript size and the relative amounts of the biomarker proteins of the invention or any control gene product, in accordance with conventional Northern hybridization techniques known to those persons of ordinary skill in the art. Still further embodiments demonstrating the use of immunohistochemical methods for evaluating expression value is shown in Example 5.

In yet some other embodiments, the detecting molecule/s suitable for the method of the invention may be at least one labeled or tagged C1RL, AGRN, ADIRF, ITLN1, CPM, VCAN, BCAT1, NDRG3, CD109, CDH1, ADH1B, GULP1, GLUL, CD34, CRNN, SERPINB5, CEACAM6, LGALS7, S100A14, THY1 and GLRX3 protein/s or any fragment/s, peptide/s or mixture/s thereof. In such case, the determination of the expression level of said at least one biomarker protein/s may be performed by mass spectrometry. Still further, in some alternative embodiments, the detecting molecules suitable for the invention may further include in addition to the at least one labeled or tagged C1RL, AGRN, ADIRF, ITLN1, CPM, VCAN, BCAT1, NDRG3, CD109, CDH1, ADH1B, GULP1, GLUL, CD34, CRNN, SERPINB5, CEACAM6, LGALS7, S100A14, THY1 and GLRX3, also at least one of labeled or tagged C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3.

Mass spectrometry (MS) is used herein as an analytical chemistry technique to identify the amount and type of chemicals present in a sample by measuring the mass-to-charge ratio and abundance of gas-phase ions. A mass spectrum is a plot of the ion signal as a function of the mass-to-charge ratio. The spectra are used to determine the elemental or isotopic signature of a sample, the masses of particles and of molecules, and to elucidate the chemical structures of molecules, such as peptides and other chemical compounds.

As noted above, the invention contemplates the use of Mass spectrometry-based absolute quantification assays that generally require recombinant expression of full length, labeled protein standards. Mass spectrometry is not inherently quantitative but many methods have been developed to overcome this limitation. Most of them are based on stable isotopes and introduce a mass shifted version of the peptides of interest, which are then quantified by their “heavy” to “light” ratio. Stable isotope labeling is either accomplished by chemical addition of labeled reagents, enzymatic isotope labeling, or metabolic labeling. Generally, these approaches are used to obtain relative quantitative information on protein expression levels in a light and a heavy labeled sample. For example, stable isotope labeling by amino acids in cell culture (SILAC) is performed by metabolic incorporation of light or heavy labeled amino acids into the recombinant or synthetic protein. Labeled protein can also be used as internal standards for determining expression levels of a cell or tissue protein of interest, such as in the spike-in SILAC approach. Several methods for absolute quantification have emerged over the last years and may be applicable for the present invention, including absolute quantification (AQUA), quantification concatamer (QConCAT), protein standard absolute quantification (PSAQ), absolute SILAC, and FlexiQuant. They all quantify the endogenous protein of interest by the heavy to light ratios to a defined amount of the labeled counterpart spiked into the sample and are chiefly distinguished by either spiking in heavy labeled peptides or heavy labeled full length proteins. The AQUA strategy is convenient and streamlined: proteotypic peptides are chemically synthesized with heavy isotopes and spiked in after sample preparation.

Still further, the QconCAT approach is based on artificial proteins that are concatamers of proteotypic peptides. This artificial protein is recombinantly expressed in host cells, for example, bacterial cells such as Escherichia coli and spiked into the sample before proteolysis. QconCAT in principle allows efficient production of labeled peptides but does not automatically correct for protein fractionation effects or digestion efficiency in the native proteins versus the concatamers. The PSAQ, absolute SILAC and FlexiQuant approaches sidestep these limitations by metabolically labeling full length proteins by heavy versions of the amino acids arginine and lysine. PSAQ and FlexiQuant in vitro synthesize full-length proteins in wheat germ extracts or in bacterial cell extract, respectively, whereas absolute SILAC was described with recombinant protein expression in E. coli. The protein standard is added at an early stage, such as directly to cell lysate. Consequently, sample fractionation can be performed in parallel and the SILAC protein is digested together with the proteome under investigation. Another quantitative approach applicable for the purpose of the present invention may be in some embodiments the SILAC-PrEST assay. In this method, Protein Epitope Signature Tags (PrESTs) are expressed recombinantly in E. coli and they consist of a short and unique region of the protein of interest as well as purification and solubility tags. A highly purified, stable isotope labeling of amino acids in cell culture (SILAC)-labeled version of the solubility tag is first quantified and used to determine the precise amount of each PrEST by its SILAC ratios. The PrESTs are then spiked into the examined sample (e.g., cell lysates) and the SILAC ratios of PrEST peptides to peptides from endogenous target proteins yield their cellular quantities.

In some embodiments, in the context of the present invention, the labeled or tagged biomarker/s of the invention or any labeled fragments or peptides thereof (that are used herein as detecting molecules) are mixed with the sample of with any protein extracted therefrom. The resulting protein mixture may be then digested according to the FASP protocol [Wisniewski, J. Ret al., Nat Meth 6:359-362(2009)] and the peptides are separated into fractions by anion exchange chromatography in a StageTip format [Wisniewski al., Journal of Proteome Research 8:5674-5678 (2009)]. Each fraction is analyzed by online reverse-phase chromatography coupled to high resolution, quantitative mass spectrometry analysis.

A variety of mass spectrometry systems can be employed in the methods of the invention for identifying and/or quantifying a biomarker protein of the invention or any fragment or peptide thereof in a sample. Mass analyzers with high mass accuracy, high sensitivity and high resolution include, but are not limited to, Q-Exative Plus or Q-Exactive HF mass spectrometers (ThermoFischer scientific), matrix-assisted laser desorption time-of-flight (MALDI-TOF) mass spectrometers, electrospray ionization time-of-flight (ESI-TOF) mass spectrometers, Fourier transform ion cyclotron mass analyzers (FT-ICR-MS), and Orbitrap analyzer instruments. Other modes of MS include ion trap and triple quadrupole mass spectrometers. In ion trap MS, analytes are ionized by electrospray ionization or MALDI and then put into an ion trap. Trapped ions can then be separately analyzed by MS upon selective release from the ion trap. Ion traps can also be combined with the other types of mass spectrometers described above.

Fragments can also be generated and analyzed. Reference biomarker protein/s labeled with an ICAT or VICAT or iTRAQ type reagent, or SILAC labeled peptides can be analyzed, for example, by single stage mass spectrometry with a MALDI or ESI ionization and with TOF, quadrupole, iontrap, FT-ICR or Orbitrap analyzers. Methods of mass spectrometry analysis are well known to those skilled in the art. For high resolution peptide fragment separation, liquid chromatography ESI-MS/MS or automated LC-MS/MS, can be used. MS analysis can be performed in a data-dependent manner or using targeted MS techniques such as selected reaction monitoring (SRM) or parallel reaction monitoring (PRM).

In some other embodiments, when the detecting molecules used are at least one of antibodies, nucleic acid, peptide or protein aptamers or any combination thereof, specific for said at least one of said biomarker proteins, the determination of the expression level of said biomarker protein/s may be performed by an immunological assay.

In some specific embodiments, determination of the expression level of the biomarker may be performed using ELISA. Enzyme-Linked Immunosorbent Assay (ELISA) is used herein involves fixation of a sample containing a protein substrate (e.g., fixed cells or a protein solution) to a surface such as a well of a microtiter plate. A substrate-specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy. In some specific embodiments, determination of the expression level of the biomarker may be performed using Western blot. Western Blot as used herein involves separation of a substrate from other protein by means of an acryl amide gel followed by transfer of the substrate to a membrane (e.g., nitrocellulose, nylon, or PVDF). Presence of the substrate is then detected by antibodies specific to the substrate, which are in turn detected by antibody-binding reagents. Antibody -binding reagents may be, for example, protein A or secondary antibodies. Antibody-binding reagents may be radio labeled or enzyme-linked, as described hereinafter. Detection may be by autoradiography, colorimetric reaction, or chemiluminescence. This method allows both quantization of an amount of substrate and determination of its identity by a relative position on the membrane indicative of the protein's migration distance in the acryl amide gel during electrophoresis, resulting from the size and other characteristics of the protein.

In some specific embodiments, different RIA assays may be employed for determination of the expression level of the biomarker proteins of the invention. In one version, Radioimmunoassay (RIA) involves precipitation of the desired protein (i.e., the substrate) with a specific antibody and radio labeled antibody -binding protein (e.g., protein A labeled with I¹²⁵) immobilized on a perceptible carrier such as agars beads. The radio-signal detected in the precipitated pellet is proportional to the amount of substrate bound.

In an alternate version of RIA, a labeled substrate and an unlabeled antibody-binding protein are employed. A sample containing an unknown amount of substrate is added in varying amounts. The number of radio counts from the labeled substrate-bound precipitated pellet is proportional to the amount of substrate in the added sample.

Still further, in specific embodiments, determination of the expression level of the biomarker/s of the invention may be performed using FACS. Fluorescence Activated Cell Sorting (FACS) involves detection of a substrate in situ in cells bound by substrate-specific, fluorescently labeled antibodies. The substrate-specific antibodies are linked to fluorophore. Detection is by means of a flow cytometry machine, which reads the wavelength of light emitted from each cell as it passes through a light beam. This method may employ two or more antibodies simultaneously, and is a reliable and reproducible procedure used by the present invention.

As described in Example 5, the biomarker protein signature of the invention has been also verified using immunohistochemical assays. Thus, in some specific embodiments, determination of the expression level of the biomarker may be performed using immunohistochemistry methods. Immuno histochemical Analysis involves detection of a substrate in situ in fixed cells by substrate-specific antibodies. The substrate specific antibodies may be enzyme-linked or linked to fluorophore. Detection is by microscopy, and is either subjective or by automatic evaluation. With enzyme-linked antibodies, a calorimetric reaction may be required. It will be appreciated that immunohistochemistry is often followed by counterstaining of the cell nuclei, using, for example, Hematoxyline or Giemsa stain.

It should be appreciated that all the detecting molecules used by any of the methods, as well as the compositions and kits of the invention described herein after, are isolated and/or purified molecules. As used herein, “isolated” or “purified” when used in reference to a nucleic acid (probes, primers and aptamers) means that a naturally occurring sequence has been removed from its normal cellular environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95% pure) of non-nucleotide material naturally associated with it, and thus is distinguished from isolated chromosomes. As used herein, the terms “isolated” and “purified” in the context of a proteineous agent (e.g., a peptide, polypeptide, protein or antibody) refer to a proteineous agent which is substantially free of cellular material and in some embodiments, substantially free of heterologous proteineous agents (i.e. contaminating proteins) from the cell or tissue source from which it is derived, or substantially free of chemical precursors or other chemicals when chemically synthesized. The language “substantially free of cellular material” includes preparations of a proteineous agent in which the proteineous agent is separated from cellular components of the cells from which it is isolated and/or recombinantly and/or synthetically produced. Thus, a proteineous agent that is substantially free of cellular material includes preparations of a proteineous agent having less than about 30%, 20%, 10%, or 5% (by dry weight) of heterologous proteineous agent (e.g. protein, polypeptide, peptide, or antibody; also referred to as a “contaminating protein”). When the proteineous agent is recombinantly produced, it is also preferably substantially free of culture medium, i.e. culture medium represents less than about 20%, 10%, or 5% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. Preferably, proteinaceous agents disclosed herein are isolated.

In some other alternative embodiments, the method of the invention may comprise determining the level of expression of at least one or of at least two, at least three, at least four, at least five, at least six, at least seven, at least eight, or all of said CLCA4, OVGP1, S100A14, SPRR3, RNASE3, SERPINB5, CLUAP1, CEACAM5 and ENPP3 biomarker protein/s by performing the step of subjecting a biological sample of said subject, or any protein product obtained therefrom to a mass spectrometry assay. It should be appreciated that the invention further encompasses combination of at least one or more of the indicated biomarkers of the invention with at least one additional biomarker, for example, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3, that may be also subjected to mass spectrometry assay. Thus, it should be appreciated that in certain embodiments, the signature proteins, specifically, at least one, at least two, at least three, at least four, at least five, at least six, at least seven, or at least eight or all of the biomarker proteins of the invention or any protein-fragments thereof may be also detected and quantified without the need for detection molecule/s. Detection can be based on MS approaches using non-targeted or targeted methods such as selected reaction monitoring (SRM) or parallel reaction monitoring (PRM). These analyses can be performed with or without a reference heavy standard and provide quantitative measure of the peptide/protein amount. The heavy reference can be a synthetic peptide, or a chemically labeled peptide/protein or metabolically labeled proteins. In the absence of a standard, the MS signal can provide the measure of peptide abundance.

According to some embodiments, the method of the invention may use as a sample any one of a biological sample of body fluids, organ/s, cell/s or tissue/s or a blood sample. As used herein, the term “sample” refers to cells, sub-cellular compartments thereof, tissue or organs. The tissue may be a whole tissue, or selected parts of a tissue. Tissue parts can be isolated by micro-dissection of a tissue, or by biopsy, or by enrichment of sub-cellular compartments. The term “sample” further refers to healthy as well as diseased or pathologically changed cells or tissues. Hence, the term further refers to a cell or a tissue associated with a disease, such a tumor, in particular carcinoma, ovarian cancer, and more specifically, High-grade ovarian carcinoma. A sample can be cells that are placed in or adapted to tissue culture. A sample can additionally be a cell or tissue from any mammalian species, specifically, humans. A tissue sample can be further a fractionated or preselected sample, if desired, preselected or fractionated to contain or be enriched for particular cell types.

In some specific and non-limiting embodiments, the sample of the method of the invention may be a body fluid sample. More specifically, such sample may be any body fluid such as blood, plasma, lymph, urine, saliva, serum, cerebrospinal fluid, seminal plasma, pancreatic juice, breast milk, uterine or lung lavage. More specifically, the sample may be uterine lavage sample. The sample can be fractionated or preselected by a number of known fractionation or pre selection techniques. A sample can also be any extract of the above. The term also encompasses protein fractions or alternatively, nucleic acid from cells or tissue. Thus, in some specific embodiments, the sample may be any one of a biological sample of organ/s, cell/s or tissue/s and a blood sample. In yet some other embodiments, the sample may be a primary tumor sample. In certain embodiments, the sample is obtained from a subject suffering from ovarian cancer.

Fractionation of samples by isolation of microvesicles was proved by the inventors to be an efficient strategy in order to enhance the throughput of MS analysis for identification of low expressing biomarkers [17].

Thus, in some further specific embodiments, the sample of the method of the invention may be microparticles/ microvesicles prepared from said body fluid.

It should be therefore appreciated that the invention provides in some embodiments thereof, a method that may further comprise at least part of the step of isolating microparticles/microvesicles from said body fluid sample, as well as at least part of the steps of isolating the sample. These procedures are described in more detailed herein below, as well as in the Experimental procedures section.

In yet more specific embodiments, the invention further provides a method comprising the following steps. The first step (a), isolating microparticles/microvesicles from at least one body fluid sample of a subject. The next step (b), involves determining the expression level of at least one biomarker protein in the microparticles/microvesicles prepared from the sample of said subject, to obtain an expression value for each of said at least one biomarker protein/s. In more specific embodiments, the said at least one, at least two, at least three, at least four, at least five, at least six, at least seven, or at least eight or all, biomarker proteins may be selected from CLCA4, OVGP1, S100A14, SPRR3, RNASE3, SERPINB5, CLUAP1, CEACAM5 and ENPP3, or any combination thereof, or optionally any combinations thereof with any additional biomarkers, for example, at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3. In the next step (c), determining if the expression value obtained in step (b) for each of said at least one biomarker protein/s is positive or negative with respect to a predetermined standard expression value or to an expression value of said biomarker protein/s in at least one control sample. In some embodiments, wherein at least one of (i) a positive expression value of at least one of said SPRR3, SERPINB5, CEACAM5, S100A14 and CLCA4 biomarker protein/s in said sample, indicates that said subject belongs to a predetermined population suffering from ovarian cancer. In other words, a high expression of these biomarkers, specifically when compared to healthy controls, indicates that the subject is diagnosed by the methods of the invention as an ovarian cancer patient. Still further, (ii) a negative expression value of at least one of said OVGP1, CLUAP1, ENPP3 and RNASE3 biomarker protein/s in said sample, indicates that said subject belongs to a predetermined population suffering from ovarian cancer. In other words, low expression of the specific biomarkers that is lower than the expression in the healthy controls, indicates that the patient can be diagnosed as affected by ovarian cancer.

Still further, in some specific embodiments, in addition to the nine-signatory biomarkers as used by the invention, when at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3 is also used, a positive expression value of at least one of CEACAM6, LGALS7, BCAT1, ADIRF, S100A14, CRNN, AGRN, ADH1B, CDH1, GLUL and SERPINB5, and a negative expression value of at least one of THY1, GLRX3, VCAN, CPM, CD34, CD109, ITLN1, C1RL, GULP1 and NDRG3 biomarker protein/s in said sample, indicates that said subject belongs to a predetermined population suffering from ovarian cancer, specifically, that the subject is affected by ovarian cancer.

As indicated herein and exemplified by the invention, microvesicles are prepared from the body fluid sample. The terms “microvesicles” or “microparticles” are herein used interchangeably and refers to are large vesicles (100 nm-1 μm), which protrude directly from the plasma membrane. These terms also encompass “exosome” which refer to smaller vesicles (40-100 nm) that originate from endocytic compartments known as the multivesicular endosomes. These microvesicles are constitutively shed from all cell types into the blood, carrying a proteomic signature of their cells of origin. Microparticles mediate local and systemic communication in various conditions, in particularly in cancer, where they can promote metastasis, immune evasion of cancer cells and angiogenesis, but also in other conditions including autoimmune diseases and cardiovascular disorders. Therefore, circulating plasma microparticle proteomics can reveal biomarkers of various diseases as the basis for further diagnostic test development.

In some specific and non-limiting embodiments, the step of isolating microvesicles may be performed by high speed centrifugation (20,000×g) of sample for 1 hour at 4° C. following by a washing step with PBS solution and additional high speed centrifugation (20,000×g for 1 hour at 4° C.). Solubilization of the microparticle pellet may be performed in lysis buffer containing 6M urea, 2M thiourea in 50 mM ammonium bicarbonate. Additional protocols for isolation of microvesicles are also available in the literature as for example Owen et al. (Owen et al., J Immunol Methods. 375: 207-214 (2012)), and are therefore applicable in the present invention. Kits for exosome isolation are commercially available and include for example ME™ Kit for Exosome Isolation (New England Peptide, Inc). It should be therefore appreciated that the invention further encompasses the use of any of the methods and kits for isolating microparticles from the body fluid sample.

Devices for analysis of microvesicles/exosomes from clinical sample are also commercially available as for example ExosomeDx (Exosome Diagnostics C)).

In some embodiments, the body fluid employed for the method of the invention may be at least one of uterine lavage fluid (UtLF) and plasma.

The term “uterine lavage fluid (UtLF)” as used herein refers to a fluid obtained through a process where a small amount of fluid (saline solution) is slowly infused into the uterine cavity and fallopian tubes and immediately retrieved.

As used herein the term “plasma” refers to blood plasma, i.e. a straw colored liquid component of blood that holds the blood cells in whole blood in suspension; plasma thus represents the extracellular matrix of blood cells. It makes up about 55% of the body's total blood volume. It is the intravascular fluid part of extracellular fluid (all body fluid outside of cells). It is mostly composed of water (up to 95% by volume), and contains dissolved proteins (6-8%) (i.e.—serum albumins, globulins, and fibrinogen), glucose, clotting factors, electrolytes (Na+, Ca2+, Mg2+, HCO3-, Cl—, etc.), hormones, carbon dioxide (plasma being the main medium for excretory product transportation) and oxygen. Plasma also serves as the protein reserve of the human body. Sampling via the uterine lavage (UtL) approach has several benefits for detection of ovarian cancer, making it highly feasible: the technique does not require previous training, equipment, imaging or sedation. The intrauterine insemination catheter can be easily inserted in daily clinic settings, even in nulliparous women. The sample processing is neither expensive nor labor-intensive and it does not require any distinctive skills or resources. A uterine lavage sample contains cells or their secreted biological products (i.e. proteins, cell-free RNA and DNA) from the lower reproductive tract. The inventors suggest herein that analysis of locally secreted molecules may have advantages over serum analysis for detecting early-stage lesions biomarkers.

Thus, in some embodiments, the sample used in method of the invention may comprise microvesicles isolated from UtLF.

As showed herein, by combining proteomic analysis from microvesicles isolated from uterine lavage samples, the inventors were able to identify the 9 biomarker protein as listed in Table 4.

In yet other embodiments, the ovarian cancer diagnosed by the method of the invention may be high-grade ovarian carcinoma (HGOC).

In another embodiment, the method of the invention may enable early detection of HGOC in a subject.

As detailed in Example 1, the patients that were chosen in order to look for biomarker of ovarian cancer as described in the present invention were suffering from late stage High-grade ovarian cancer. However, it is suggested by Examples 3, 4 and 5, that these 9 biomarkers enable detection at early stage of ovarian cancer. An “early diagnosis” or “early detection” may be used interchangeably, and provides diagnosis prior to appearance of clinical symptoms. Prior as used herein is meant days, weeks, months or even years before the appearance of such symptoms. More specifically, at least 1 week, at least 1 month, 2 months, 3 months, 4 months, 5 months, 6 months, 7 months, 8 months, 9 months, 10 months, 11 months, 12 months, or even few years before clinical symptoms appear.

It should be appreciated that the method of the invention may be suitable for any mammalian subject. By “patient” or “subject” it is meant any mammal that may be affected by the above-mentioned conditions, and to whom the treatment and diagnosis methods herein described is desired, including human, bovine, equine, canine, murine and feline subjects. Specifically, said subject is a human. Thus, in yet some further embodiments, the methods of the invention may be suitable for any mammalian female subject, specifically to any woman. In yet some further embodiments, the methods and kits of the invention may be suitable for any woman aged between 12 years to 90 or older. In yet some further embodiments, the methods and kits of the invention may be suitable for early diagnosis of ovarian carcinoma in any woman over 30, 35, 40, 45, 50, 55, 60, 65, 70 years old, or even older.

In some specific and non-limiting embodiments, the method of the invention may be suitable for subjects that belong to a high-risk population. In some particular embodiments, such subject may be subject carrying at least one mutation in at least of BRCA1 and BRCA2 genes. High-risk population are women with mutations in the genes BRCA1 or BRCA2 that have about a 50% chance of developing the disease. The mutation in BRCA1 or BRCA2 DNA mismatch repair genes is present in 10% of ovarian cancer cases. Only one allele need be mutated to place a person at high risk, because the risky mutations are autosomal dominant. The gene can be inherited through either the maternal or paternal line, but has variable penetrance. Though mutations in these genes are usually associated with increased risk of breast cancer, they also carry a substantial lifetime risk of ovarian cancer, a risk that peaks in a woman's 40s and 50s. The lowest risk cited is 30% and the highest 60%. Mutations in BRCA1 have a lifetime risk of developing ovarian cancer of 15-45%. Mutations in BRCA2 are less risky than those with BRCA1, with a lifetime risk of 10% (lowest risk cited) to 40% (highest risk cited). On average, BRCA-associated cancers develop 15 years before their sporadic counterparts, because people who inherit the mutations on one copy of their gene only need one mutation to start the process of carcinogenesis, whereas people with two normal genes would need to acquire two mutations. In some embodiments, for subjects classified as patients suffering from ovarian cancer by the methods of the invention, an endocrine therapy or any combination thereof with a biological therapy may be offered. Endocrine therapy refers to a treatment that adds, blocks, or removes hormones. In the context of the present disclosure, endocrine therapy is provided to slow or stop the growth of ovarian cancers. In this connection, synthetic hormones or other drugs may be given to block the body's natural hormones. In yet some further embodiments, therapy based on aromatase inhibitors may be offered. Other therapeutic options may also include biological therapy (antibodies and the like) and cryotherapy. In yet some other embodiments, where the subject is classified as an ovarian cancer suffering patient, chemotherapy, radiotherapy or any combinations thereof may be offered. Thus, in some alternative and optional embodiments, the methods of the invention may further comprise the step of administering to a subject diagnosed as suffering from ovarian cancer, a therapeutically effective amount of a therapeutic agent, specifically, any synthetic hormone, aromatase inhibitor, chemotherapeutic agent and/or biological therapy agent, or any combinations thereof. Alternatively or additionally, the method may comprise in some embodiments, the step of subjecting a subject diagnosed with ovarian cancer, to at least one of endocrine therapy, chemotherapy, radiotherapy, biological therapy (antibodies and the like), cryotherapy, and any combinations thereof In more specific embodiments, such therapeutic agent may be an endocrine agent, specifically, synthetic hormones, aromatase inhibitors.

The invention therefore offers in some aspects thereof therapeutic methods for treating subjects suffering from ovarian cancer, comprising the steps of:

In a first step, determining the expression level of at least one, at least two, at least three, at least four, at least five, at least six, at least seven, at least eight, at least nine, specifically, at least three biomarker protein/s in at least one biological sample of said subject, to obtain an expression value for each of said at least one biomarker protein/s, wherein said at least one biomarker proteins are selected from CLCA4, OVGP1, S100A14, SPRR3, RNASE3, SERPINB5, CLUAP1, CEACAM5 and ENPP3 protein/s or any combination thereof. It should be understood that this step is as described herein in connection with the diagnostic methods of the invention. The second step involves determining if the expression value obtained in step (a) for each of the at least one biomarker protein/s is positive or negative with respect to a predetermined standard expression value or to an expression value of said biomarker protein/s in at least one control sample. It should be noted that at least one of: (i) a positive expression value of at least one of said SPRR3, SERPINB5, CEACAM5, S100A14 and CLCA4 biomarker protein/s in the sample, indicates that the subject suffers from ovarian cancer; and (ii) a negative expression value of at least one of said OVGP1, CLUAP1, RNASE3 and ENPP3 biomarker protein/s in said sample, indicates that the subject suffers from ovarian cancer.

The next step involves administering to a subject diagnosed as suffering from ovarian cancer, a therapeutically effective amount of at least one therapeutic agent, specifically, any synthetic hormone, aromatase inhibitor, chemotherapeutic agent and/or biological therapy agent, or any combinations thereof. Alternatively or additionally, the method may comprise in some embodiments, the step of subjecting a subject diagnosed with ovarian cancer, to at least one of endocrine therapy, chemotherapy, radiotherapy, biological therapy (antibodies and the like), cryotherapy, and any combinations thereof.

In yet a further aspect, the invention relates to a diagnostic composition comprising at least one detecting molecule or any combination or mixture of plurality of detecting molecules specific for determining the level of expression of at least one of CLCA4, OVGP1, S100A14, SPRR3, RNASE3, SERPINB5, CLUAP1, CEACAM5 and ENPP3 protein/s or any combination thereof, wherein each of said detecting molecules is specific for one of said biomarker protein/s. Still further, in some additional embodiments, the composition of the invention may further comprise at least one detecting molecule or any combination or mixture of plurality of detecting molecules specific for determining the level of expression of at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3.

It should be noted that each of said detecting molecules is specific for one of said biomarker proteins. It should be appreciated that in certain embodiments, the composition of the invention may be at least one of diagnostic composition. In certain embodiments, the detecting molecules comprised within the composition of the invention may be attached to a solid support. Definitions of solid support that may be used as part of the diagnostic composition of the invention are described in more detail herein after, in connection with the kit of the invention. It should be appreciated that in some specific and non-limiting embodiments, the detecting molecules of the composition of the invention may be provided in a suitable medium or a buffer. In some alternative embodiments, the detecting molecules of the invention may be provided in a dried form.

It should be appreciated that the invention encompasses compositions comprising detecting molecules specific for any combination of any of the marker protein used by the invention. In some embodiment, the composition of the invention may comprise at least one detecting molecule or any combination or mixture of plurality of detecting molecules specific for determining the level of expression of at least two of CLCA4, OVGP1, S100A14, SPRR3, RNASE3, SERPINB5, CLUAP1, CEACAM5 and ENPP3 protein/s or any combination thereof, wherein each of said detecting molecules is specific for one of said biomarker proteins.

It should be noted that in some embodiments, each of the detecting molecules is specific for one of said biomarker proteins. In some particular and non-limiting embodiments of the invention, such at least two of CLCA4, OVGP1, S100A14, SPRR3, RNASE3, SERPINB5, CLUAP1, CEACAM5 and ENPP3 biomarker protein/s may comprise S100A14 and SERPINB5. It should be appreciated that in some embodiments, the two biomarker proteins may further comprise at least one, at least two, at least three, at least four, at least five, at least six, at least seven, of the CLCA4, OVGP1, SPRR3, RNASE3, CLUAP1, CEACAM5 and ENPP3 biomarker proteins of the invention.

In some embodiment, the composition of the invention may comprise at least one detecting molecule or any combination or mixture of plurality of detecting molecules specific for determining the level of expression of at least two of CLCA4, OVGP1, S100A14, SPRR3, RNASE3, SERPINB5, CLUAP1, CEACAM5 and ENPP3 protein/s or any combination thereof, wherein each of said detecting molecules is specific for one of said biomarker proteins.

It should be noted that in some embodiments, each of the detecting molecules is specific for one of said biomarker proteins. In some particular and non-limiting embodiments of the invention, such at least two of CLCA4, OVGP1, S100A14, SPRR3, RNASE3, SERPINB5, CLUAP1, CEACAM5 and ENPP3 biomarker protein/s may comprise S100A14 and CLCA4. It should be appreciated that in some embodiments, the two biomarker proteins may further comprise at least one, at least two, at least three, at least four, at least five, at least six, at least seven, of the OVGP1, SPRR3, RNASE3, SERPINB5, CLUAP1, CEACAM5 and ENPP3 biomarker proteins of the invention. In another embodiment, the composition of the invention may comprise at least one detecting molecule or any combination or mixture of plurality of detecting molecules specific for determining the level of expression of at least three of CLCA4, OVGP1, S100A14, SPRR3, RNASE3, SERPINB5, CLUAP1, CEACAM5 and ENPP3 protein/s or any combination thereof, wherein each of said detecting molecules is specific for one of said biomarker proteins. It should be noted that in some embodiments, each of the detecting molecules is specific for one of said biomarker proteins. In some particular and non-limiting embodiments of the invention, such at least three of CLCA4, OVGP1, S100A14, SPRR3, RNASE3, SERPINB5, CLUAP1, CEACAM5 and ENPP3 biomarker protein/s may comprise CLCA4, OVGP1 and S100A14. It should be appreciated that in some embodiments, the three biomarker proteins may further comprise at least one, at least two, at least three, at least four, at least five, at least six, of the SPRR3, RNASE3, SERPINB5, CLUAP1, CEACAM5 and ENPP3biomarker proteins of the invention.

In further embodiments, the composition of the invention may comprise at least one detecting molecule or any combination or mixture of plurality of detecting molecules specific for determining the level of expression of at least four of CLCA4, OVGP1, S100A14, SPRR3, RNASE3, SERPINB5, CLUAP1, CEACAM5 and ENPP3 protein/s or any combination thereof. It should be noted that each of the detecting molecules is specific for one of said biomarker proteins. In some particular and non-limiting embodiments of the invention, such at least four of CLCA4, OVGP1, S100A14, SPRR3, RNASE3, SERPINB5, CLUAP1, CEACAM5 and ENPP3 biomarker protein/s may comprise S100A14, CLCA4, CLUAP1 and CEACAM5as also demonstrated by FIG. 2B in Example 3. It should be appreciated that in some embodiments, the four biomarker proteins may further comprise at least one, at least two, at least three, at least four, at least five, of the OVGP1, SPRR3, RNASE3, SERPINB5 and ENPP3 biomarker proteins of the invention.

In yet some further embodiments, as also demonstrated by Example 3, the at least four biomarkers may include S100A14, CLCA4, SPRR3 and SERPINB5.

In further embodiments, the composition of the invention may comprise at least one detecting molecule or any combination or mixture of plurality of detecting molecules specific for determining the level of expression of at least five of CLCA4, OVGP1, S100A14, SPRR3, RNASE3, SERPINB5, CLUAP1, CEACAM5 and ENPP3 protein/s or any combination thereof. It should be noted that each of the detecting molecules is specific for one of said biomarker proteins. In some particular and non-limiting embodiments of the invention, such at least six of CLCA4, OVGP1, S100A14, SPRR3, RNASE3, SERPINB5, CLUAP1, CEACAM5 and ENPP3 biomarker protein/s may comprise S100A14, CLCA4, OVGP1, ENPP3 and RNASE3. It should be appreciated that in some embodiments, the five biomarker proteins may further comprise at least one, at least two, at least three, at least four, of the SPRR3, SERPINB5, CLUAP1 and CEACAM5 biomarker proteins of the invention.

In further embodiments, the composition of the invention may comprise at least one detecting molecule or any combination or mixture of plurality of detecting molecules specific for determining the level of expression of at least six of CLCA4, OVGP1, S100A14, SPRR3, RNASE3, SERPINB5, CLUAP1, CEACAM5 and ENPP3 protein/s or any combination thereof. It should be noted that each of the detecting molecules is specific for one of said biomarker proteins. In some particular and non-limiting embodiments of the invention, such at least six of CLCA4, OVGP1, S100A14, SPRR3, RNASE3, SERPINB5, CLUAP1, CEACAM5 and ENPP3 biomarker protein/s may comprise OVGP1, CLCA4, S100A14, CLUAP1, SERPINB5 and ENPP3. It should be appreciated that in some embodiments, the six biomarker proteins may further comprise at least one, at least two, at least three, of the SPRR3, RNASE3 and CEACAM5 biomarker proteins of the invention.

In some particular and non-limiting embodiments of the invention, such at least six of CLCA4, OVGP1, S100A14, SPRR3, RNASE3, SERPINB5, CLUAP1, CEACAM5 and ENPP3 biomarker protein/s may comprise SERPINB5, S100A14, OVGP1, CLCA4, CLAUP1 and CEACAM5. It should be appreciated that in some embodiments, the six biomarker proteins may further comprise at least one, at least two, at least three, of the SPRR3, RNASE3, and ENPP3 biomarker proteins of the invention.

In further embodiments, the composition of the invention may comprise at least one detecting molecule or any combination or mixture of plurality of detecting molecules specific for determining the level of expression of at least seven of CLCA4, OVGP1, S100A14, SPRR3, RNASE3, SERPINB5, CLUAP1, CEACAM5 and ENPP3 protein/s or any combination thereof. It should be noted that each of the detecting molecules is specific for one of said biomarker proteins. In some particular and non-limiting embodiments of the invention, such at least seven of CLCA4, OVGP1, S100A14, SPRR3, RNASE3, SERPINB5, CLUAP1, CEACAM5 and ENPP3 biomarker protein/s may comprise CEACAM5, RNASE3, SERPINB5, OVGP1, CLCA4, S100A14, SPRR3 (according to FIG. 13 in Example 6). It should be appreciated that in some embodiments, the seven biomarker proteins may further comprise at least one, at least two of the CLUAP1, and ENPP3 biomarker proteins of the invention. Other specific embodiments for at least seven and at least eight of the biomarkers of the invention are described in detail in connection with the methods of the invention and are applicable for the current aspect as well.

In certain embodiments, the compositions of the invention may further comprise detecting molecules specific for control reference protein. Such control reference protein may be used for normalizing the detected expression levels for the biomarker proteins used by the invention. Non-limiting embodiments for control reference proteins may include Actin, Talin (TLN1), Vinculin (VCL) or other proteins.

It should be appreciated that the composition of the invention may comprise at least one detecting molecules specific for at least one biomarker of the invention, specifically, at least 1, 2, 3, 4, 5, 6, 7, 8 or 9of the biomarkers of Table 4, specifically, CLCA4, OVGP1, S100A14, SPRR3, RNASE3, SERPINB5, CLUAP1, CEACAM5 and ENPP3. In yet some further embodiments, in addition, the composition of the invention may comprise detecting molecules specific for at least one further additional biomarker, specifically, at least 1, 2, 3, 4, 5, 6, 7, 8, 9, 10, 11, 12, 13, 14, 15, 16, 17, 18 or 19 of the C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3 biomarkers. In some embodiments, the composition of the invention may comprise detecting molecules specific for at least one further additional biomarker. In more specific embodiments, the compositions of the invention may comprise also detecting molecule/s specific for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200, 250, 300, 350, 384, 400, 450 and 500 at the most, additional biomarker proteins.

According to some embodiments, the detecting molecules suitable for the composition of the invention may be selected from amino acid detecting molecules and nucleic acid detecting molecules.

In yet some specific embodiments, the amino acid detecting molecules suitable for the composition of the invention may comprise at least one of: (a) at least one labeled or tagged CLCA4, OVGP1, S100A14, SPRR3, RNASE3, SERPINB5, CLUAP1, CEACAM5 and ENPP3 protein/s or any fragment/s, peptide/s or mixture/s thereof; (b) at least one antibody specific for said at least one of said biomarker protein/s; (c) at least one peptide aptamer/s specific for said at least one biomarker protein/s; and (d) any combination of (a), (b) and (c).

It should be noted that any of the amino acid based detecting molecules described herein before for the methods of the invention are also applicable for any of the compositions of the invention and are therefore encompassed by the present aspect as well.

In some further embodiments, the nucleic acid detecting molecule suitable for the composition of the invention may comprise at least one of: a) at least one nucleic acid aptamer/s specific for said at least one biomarker proteins; b) at least one oligonucleotide/s, each oligonucleotide specifically hybridizes to a nucleic acid sequence encoding said at least one biomarker protein/s.

In certain embodiments, the detecting molecules of the composition of the invention may be attached to a solid support, thus, in certain embodiments, the detecting molecules used by the invention may be immobilized or in immobilized form. More specifically, as defined herein, the detecting molecules are optionally attached to a support where each of the detecting molecules is attached to a support in a unique pre-selected and defined region. In some other embodiments, the detecting molecules may be provided in non-immobilized form, specifically, not attached to a solid support but separated in different vessels, tubes, wells and the like. Nevertheless, in yet some alternative embodiments, the detecting molecules may be provided in a mixture that contains variety of detecting molecules each specific for at least one of the biomarker proteins of the invention, and in any case detecting molecules specific for 500 at the most, biomarker proteins and control references.

In yet some other embodiments, the detecting molecules of the composition of the invention may be provided in a mixture.

It should be noted that in some embodiments, the invention provides a composition that further comprise at least one biological sample.

Thus, the invention may further comprise a composition comprising at least one of the detecting molecules specific for at least one biomarker protein/s of the invention, specifically, the biomarkers of Table 4, and a sample, specifically, a biological sample. It should be noted that in addition to the biomarker/s of Table 4, the composition of the invention may comprise detecting molecules specific for at least one further biomarker, provided that the detecting molecules of the compositions of the invention are specific for 499 biomarkers at the most.

It should be appreciated that in more specific embodiments, the compositions of the invention may comprise detecting molecules specific for at least one additional biomarker protein, specifically, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200, 250, 300, 350, 400, 450 and 500 at the most, additional biomarker proteins.

As noted above, it should be appreciated that any of the compositions of the invention may be used for early diagnosis of ovarian carcinoma, specifically, HGOC.

In yet a further aspect, the invention relates to a kit comprising: (a) at least one detecting molecule specific for determining the level of expression of at least one of CLCA4, OVGP1, S100A14, SPRR3, RNASE3, SERPINB5, CLUAP1, CEACAM5 and ENPP3 protein/s or any combination thereof in a biological sample. It should be noted that each of said detecting molecule/s is specific for one of said biomarker proteins. In some alternative embodiments, the kit of the invention may further comprise detecting molecules specific for determining the expression of at least one of C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3. It should be noted that the kit optionally further comprises at least one of: (b) pre-determined calibration curve/s or predetermined standards providing standard expression values of said at least one biomarker/s; and (c) at least one control sample.

In some embodiments,

In yet some other alternative embodiments, the kit of the invention may comprise: a) at least one detecting molecule specific for determining the level of expression of at least two of CLCA4, OVGP1, S100A14, SPRR3, RNASE3, SERPINB5, CLUAP1, CEACAM5 and ENPP3protein/s or any combination thereof in a biological sample. Each of the detecting molecule/s may be specific for one of the biomarker proteins. The kit optionally further comprises at least one of: (b) pre-determined calibration curve/s or predetermined standard/s providing standard expression values of the at least one biomarker protein/s; (c) at least one control sample.

The invention further encompass any kit comprising detecting molecules specific for at least one, at least two, at least three, at least four, at least five, at least six, at least seven, at least eight, at least nine, of the biomarker protein/s of the invention, specifically of CLCA4, OVGP1, S100A14, SPRR3, RNASE3, SERPINB5, CLUAP1, CEACAM5 and ENPP3.

It should be further understood that the kit of the invention may comprise detecting molecules specific for any combination of the biomarker proteins of the invention, specifically the combinations specified herein above in connection with the methods and compositions aspects. It should be appreciated that each of the detecting molecule/s is specific for one of said biomarker proteins. In some embodiments, the kit of the invention may optionally further comprises at least one of: pre-determined calibration curve/s or predetermined standard/s providing standard expression values of said at least one biomarker protein/s; and at least one control sample. It should be appreciated that all the combinations disclosed herein before in connection with the compositions of the invention are also applicable for any of the kits of the invention.

In other embodiments, the detecting molecules suitable for the kit of the invention may be selected from amino acid detecting molecule/s and nucleic acid detecting molecule/s. In some embodiments, the amino acid detecting molecules suitable for the kit of the invention may comprise at least one of: a) at least one labeled or tagged CLCA4, OVGP1, S100A14, SPRR3, RNASE3, SERPINB5, CLUAP1, CEACAM5 and ENPP3 protein/s or any fragment/s, peptide/s or mixture/s thereof; b) at least one antibody specific for said at least one of said biomarker proteins; c) at least one peptide aptamer/s specific for said at least one of said biomarker protein/s; d) any combination of (a), (b) and (c).

In some specific embodiments, the nucleic acid detecting molecule suitable for the kit of the invention may comprise at least one of: a) at least one nucleic acid aptamer/s specific for said at least one biomarker proteins; b) at least one oligonucleotides, each oligonucleotide specifically hybridizes to a nucleic acid sequence encoding said at least one biomarker protein/s.

In other embodiments, the detecting molecule/s used in the kit of the invention may be attached to a solid support.

The detecting molecules of the invention were described in detailed in connection with the methods of the invention. It should be appreciated that all embodiments for detecting molecules mentioned therein are also applicable for the compositions and kits of the invention.

Still further, the inventors consider the kit of the invention in compartmental form. It should be therefore noted that in certain embodiments the detecting molecules used for detecting the expression levels of the biomarker proteins may be provided in a kit attached to an array. As defined herein, a “detecting molecule array” refers to a plurality of detection molecules that may be nucleic acids based or protein based detecting molecules, optionally attached to a support where each of the detecting molecules is attached to a support in a unique pre-selected and defined region.

For example, an array may contain different detecting molecules, such as specific antibodies, labeled or tagged proteins, peptides, aptamers, probes and/or primers or any combinations thereof. As indicated herein before, in case a combined detection of the biomarker proteins expression level, the different detecting molecules for each target may be spatially arranged in a predetermined and separated location in an array. For example, an array may be a plurality of vessels (test tubes), plates, micro-wells in a micro-plate, each containing different detecting molecules, specifically, aptamers, primers and antibodies, specific for each marker protein used by the invention. An array may also be any solid support holding in distinct regions (dots, lines, columns) different and known, predetermined detecting molecules.

As used herein, “solid support” is defined as any surface to which molecules may be attached through either covalent or non-covalent bonds. Thus, useful solid supports include solid and semi-solid matrixes, such as aero gels and hydro gels, resins, beads, biochips (including thin film coated biochips), micro fluidic chip, a silicon chip, multi-well plates (also referred to as microtiter plates or microplates), membranes, filters, conducting and no conducting metals, glass (including microscope slides) and magnetic supports. More specific examples of useful solid supports include silica gels, polymeric membranes, particles, derivative plastic films, glass beads, cotton, plastic beads, alumina gels, polysaccharides such as Sepharose, nylon, latex bead, magnetic bead, paramagnetic bead, super paramagnetic bead, starch and the like. This also includes, but is not limited to, microsphere particles such as Lumavidin or LS-beads, magnetic beads, charged paper, Langmuir-Blodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

It should be further appreciated that any of the reagents, substances or ingredients included in any of the methods and kits of the invention may be provided as reagents embedded, linked, connected, attached, placed or fused to any of the solid support materials described above.

In certain embodiments, the detecting molecule/s used in the kit of the invention may be provided in a mixture. In some alternative embodiments, the detecting molecules may be provided as molecules that are not attached to any solid support. In some embodiments, the non-attached detecting molecules may be provided in separate containers, wells, tube vessels and the like. In some alternative embodiments, the attached or non-attached detecting molecules may be provided in a mixture that contains at least two detecting molecules specific for at least two biomarker protein/s of the invention.

It should be understood that any of the detecting molecules described by the invention are also applicable for this aspect.

In other embodiments, the kit of the invention may further comprise instructions for use. Such instructions may comprise at least one of: (a) instructions for carrying out the detection and quantification of the expression of said at least one of said CLCA4, OVGP1, S100A14, SPRR3, RNASE3, SERPINB5, CLUAP1, CEACAM5 and ENPP3 and optionally of at least one of the C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3 biomarker protein/s and optionally, of a control reference protein; and (b) instructions for determining if the expression values of at least one of CLCA4, OVGP1, S100A14, SPRR3, RNASE3, SERPINB5, CLUAP1, CEACAM5 and ENPP3 and optionally at least one of the C1RL, AGRN, ADIRF, ITLN1, CPM, VCAN, BCAT1, NDRG3, CD109, CDH1, ADH1B, GULP1, GLUL, CD34, CRNN, CEACAM6, LGALS7, THY1 and GLRX3, is positive or negative with respect to a corresponding predetermined standard expression value or with expression value of at least one of the biomarker protein/s in said at least one control sample.

It should be appreciated that the components in the kit may depend on the method of detection and are not limited to any method. In some embodiments, the kit of the invention may further comprise at least one reagent for conducting a mass spectrometry assay. Such reagents may include trypsin, buffers, filters and the like, for peptide purification.

In some other embodiments, the kit of the invention further comprising at least one reagent for conducting an immunological assay selected from protein microarray analysis, ELISA, RIA, slot blot, dot blot, FACS, western blot, immunohistochemical assay, immunofluorescent assay and a radio-imaging assay.

In further embodiments, the kit of the invention may further comprise at least one device, means or any reagent for obtaining a body fluid sample, specifically UtL and for isolating microparticles/ microvesicles from said body fluid sample.

In more specific embodiment, the additional reagent comprised in the kit of the invention may be lysis buffer containing 6M urea, 2M thiourea in 50 mM ammonium bicarbonate, as well as device such as catheter and the like.

In some other embodiments, the kit of the invention may be for use in a method for detecting ovarian cancer in a subject.

In certain embodiments, the kit of the invention may be suitable for use in a method for detecting High-grade ovarian carcinoma.

In yet another embodiment, the kit of the invention may be suitable for or adapted for use in a method of early detection of High-grade ovarian carcinoma. By adapted for use, is meant herein that the kit of the invention may further contain at least one means or reagent/s required for performing the diagnostic method of the invention.

In accordance with some other embodiments, the sample to be used is any one of a biopsy of organs or tissues and a blood sample. Still further, according to certain embodiments, the kits of the invention may use any appropriate biological sample. The term “biological sample” in the present specification and claims is meant to include samples obtained from a mammalian subject.

In some embodiments, the biological sample may be a bodily fluid, a tissue, a tissue biopsy, a skin swab, an isolated cell population or a cell preparation.

In some specific embodiments, the population of cells comprises cancer cells. In another embodiment the population of cells is an in vitro cultured cell population.

In some embodiments, the biological sample may be a bodily fluid selected from the group consisting of blood, serum, plasma, urine, cerebrospinal fluid, amniotic fluid, tear fluid, nasal wash, mucus, saliva, sputum, broncheoalveolar fluid, throat wash, vaginal fluid and semen. In a specific embodiment, the biological sample is uterine lavage sample.

According to an embodiment of the invention, the sample may be a tissue sample or blood sample which can be obtained using a syringe needle for example from a vein of the subject or from the tissue. It should be noted that the cell may be isolated from the subject (e.g., for in vitro detection) or may optionally comprise a cell that has not been physically removed from the subject (e.g., in vivo detection).

In certain embodiments, the sample used in the kit of the invention may be a body fluid sample. The kits of the invention may therefore further comprise any suitable means or device for obtaining said sample.

In yet another embodiment, the sample used for the kit of the invention may be microvesicles prepared from said body fluid.

In certain embodiments, the body fluid suitable for the kit of the invention may be at least one of UtLF and plasma.

In some embodiments, the sample suitable for the kit of the invention may comprise microvesicles isolated from UtLF.

One of the challenges associated with cancer and specifically ovarian cancer treatment originates from non-efficient treatments or resistance to treatment. Thus, the present invention further provides the use of at least one of the biomarker proteins as markers for evaluating response of patients treated with a certain therapeutic agent or monitoring the efficacy of treatment with a certain therapeutic agent. In some embodiments, the method of the invention may be particularly suitable for monitoring and early diagnosis of response of the diagnosed disorder in the subject.

In yet some further aspect, the invention may further provides a method for monitoring the efficacy of a treatment with a therapeutic agent and the disease progression. The method comprises the steps of: (a) determining the expression level of at least one biomarker protein in a biological sample of said subject, to obtain an expression value for each of said at least one biomarker protein/s, wherein said biomarker protein/s are selected from said CLCA4, OVGP1, S100A14, SPRR3, RNASE3, SERPINB5, CLUAP1, CEACAM5 and ENPP3 or any combination thereof; (b) repeating step (a) to obtain expression values of said at least one biomarker protein/s, for at least one more temporally-separated test sample. It should be noted that wherein at least one of said temporally separated samples is obtained after the initiation of said treatment. The next step (c) involves calculating the rate of change of said expression values of said at least one biomarker protein between said temporally-separated test samples. In the next step (d), determining if the rate of change obtained in step (c) is positive or negative with respect to a predetermined standard rate of change determined between at least two temporally separated samples or to the rate of change calculated for expression values in at least one control sample obtained from at least two temporally separated samples. It should be noted that, wherein at least one sample of said at least two samples is obtained after the initiation of said treatment. In more specific embodiments, wherein at least one of: (i) a positive rate of change of the expression value of at least one of said OVGP1, CLUAP1, RNASE3 and ENPP3 biomarker protein/s in said sample, indicates that said subject exhibits a beneficial response to said treatment; and (ii) a negative rate of change of at least one of said SPRR3, SERPINB5, CEACAM5, S100A14, CLCA4 and biomarker protein/s in said sample, indicates that said subject exhibits a beneficial response to said treatment. Simply put, elevated expression of biomarkers that display low expression in ovarian cancer patients, may indicate that the subject may respond to the treatment. Reduction in the expression of biomarkers that are overexpressed in ovarian cancer patients indicates that the subject may be classified as a responder.

It should be understood that the prognostic and monitoring methods offered by the invention may be applicable for patients that are treated with any therapeutic compound. In more specific embodiments, such patient has not been subjected to RRBSO, or any surgical intervention.

The therapy according with the present invention may be any therapy applicable to cancer and specifically to ovarian cancer. In some embodiments, for subjects classified as patients suffering from ovarian cancer by the methods of the invention, an endocrine therapy or any combination thereof with a biological therapy may be offered. Endocrine therapy refers to a treatment that adds, blocks, or removes hormones. In the context of the present disclosure, endocrine therapy is provided to slow or stop the growth of ovarian cancers. In this connection, synthetic hormones or other drugs may be given to block the body's natural hormones. In yet some further embodiments, therapy based on aromatase inhibitors may be offered. Other therapeutic options may also include biological therapy (antibodies and the like) and cryotherapy. In yet some other embodiments, where the subject is classified as an ovarian cancer suffering patient, chemotherapy, radiotherapy or any combinations thereof may be offered.

As detailed herein, the method of the invention may be also applicable for evaluating or monitoring the responsiveness of a patient, specifically a patient that was not subjected to RRBSO, to treatment with any therapeutic agent or regimen. Accordingly, the patient may be evaluated in at least one time point after initiation of treatment in order to assess if the treatment protocol is efficient and appropriate. Determination can be carried out at an early time points such that a decision may be made regarding continuation of the treatment or alternatively readjusting the treatment protocol.

In yet some other embodiments, the invention further provides a method for assessing responsiveness of a mammalian subject to treatment with a specific therapeutic agent or evaluating and/or monitoring the efficacy of treatment on a subject. This method is based on determining the expression values of the biomarkers of the invention before and any time after initiation of treatment, and calculating the ratio of the change in said values as a result of the treatment.

As indicated above, in accordance with some embodiments of the invention, in order to assess the patient condition, or monitor the disease progression, as well as responsiveness to a certain treatment, at least two “temporally-separated” test samples must be collected from the examined patient and compared thereafter in order to obtain the rate of change in the expression value of at least one of the biomarker proteins between said samples. In practice, to detect a change in at least one of these parameters between said samples, at least two “temporally-separated” test samples and preferably more must be collected from the patient.

The expression value is then determined using the method of the invention, applied for each sample. As detailed above, the rate of change in parameters is calculated by determining the ratio between at least two values of expression obtained from the same patient in different time-points or time intervals.

This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least three days, at least one week, at least two weeks, at least three weeks, at least one month, at least two months, at least three months, at least four months, at least five months, at least one year, or even more.

In some embodiments, one of the time points may correspond to a period in which a patient is experiencing a remission of the disease.

When calculating the rate of change, one may use any two samples collected at different time points from the patient. To ensure more reliable results and reduce statistical deviations to a minimum, averaging the calculated rates of several sample pairs is preferable. A calculated or average value of a negative rate of change of the expression value of at least one of said biomarker protein/s indicates that said subject exhibits a beneficial response to said treatment; thereby monitoring the efficacy of a treatment with a therapeutic agent and the disease progression. It should be noted that in certain embodiments, where normalization step is being performed, the values referred to above, are normalized values.

As indicated above, the invention provides diagnostic and prognostic methods. “Prognosis” is defined as a forecast of the future course of a disease or disorder, based on medical knowledge. This highlights the major advantage of the invention, namely, the ability to predict progression of the disease, based on the expression value of at least one of the biomarker proteins. More specifically, the ability to determine at early stage that the subject is suffering from ovarian cancer, or in some specific embodiments, HGOC. This ability facilitates the selection of appropriate treatment regimen/s that may minimize side effects from unnecessary treatment, particularly, surgical intervention, individually to each patient, as part of personalized medicine. Still further, as indicated above, in order to execute the prognostic method of the invention, at least two different samples must be obtained from the subject in order to calculate the rate of change in the expression as detailed above. By obtaining at least two and preferably more biological samples from a subject and analyzing them according to the method of the invention, the prognostic method may be effective for predicting, monitoring and early diagnosing molecular alterations indicating response to treatment in said patient.

Thus, the prognostic method may be applicable for early, sub- symptomatic diagnosis of relapse when used for analysis of more than a single sample along the time-course of diagnosis, treatment and follow-up.

The number of samples collected and used for evaluation of the subject may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. Furthermore, to assess the trend in expression rates according to the invention, it is understood that the rate of change may be calculated as an average rate of change over at least three samples taken in different time points, or the rate may be calculated for every two samples collected at adjacent time points. It should be appreciated that the sample may be obtained from the monitored patient in the indicated time intervals for a period of several months or several years. More specifically, for a period of 1 year, for a period of 2 years, for a period of 3 years, for a period of 4 years, for a period of 5 years, for a period of 6 years, for a period of 7 years, for a period of 8 years, for a period of 9 years, for a period of 10 years, for a period of 11 years, for a period of 12 years, for a period of 13 years, for a period of 14 years, for a period of 15 years or more. In one particular example, the samples are taken from the monitored subject every two months for a period of 5 years.

The method for monitoring disease progression or early prognosis for disease relapse as detailed herein may be used for personalized medicine, by collecting at least two samples from the same patient at different stages of the disease.

All scientific and technical terms used herein have meanings commonly used in the art unless otherwise specified. The definitions provided herein are to facilitate understanding of certain terms used frequently herein and are not meant to limit the scope of the present disclosure. The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Thus, as used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention.

Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

Reference is now made to the following examples, which together with the above descriptions illustrate the invention in a non-limiting fashion.

### Experimental Procedures

Patient Selection

Samples were prospectively collected in accordance with approvals of the institutional ethics committees at Chaim Sheba Medical Center, Rabin Medical Center and Meir Medical Center, Israel (ClinicalTrials.gov identifier: NCT03150121). Informed consent was obtained from each participant. Recruited patients underwent gynecological surgical procedures under general anesthesia, including hysteroscopy, hysterectomy and/or RRBSO. Eligible indications included HGOC (primary or interval debulking), suspicious ovarian mass, risk reduction, or various other benign gynecological disorders (Table 1 and Table 2). Patients with endometrial and cervical carcinoma were excluded, as well as patients with non-HG serous ovarian tumors. Additionally, we recruited clinically healthy BRCA1/2 mutation carriers who have not undergone RRBSO (Table 1 and Table 2). Non-pregnant-only participants (non-pregnant) undergo UtL during their gynecological examination at the dedicated clinic at Sheba Medical Center.

TABLE 1
Patient characteristics for UtL samples included in the proteomic analysis.

| | | Discovery | | | Validation | | |
|---|---|---|---|---|---|---|---|
| Clinical | | set | | Age | set | | Age |
| Characteristics | | No. | (%) | (ave.) | No. | (%) | (ave.) |
| Entire cohort | | 24 | | 57.4 | 152 | | 53 |
| Patient cohort: | | 12 | 100 | 60.6 | 37 | 100 | 62.3 |
| Type of surgery: | Primary debulking | 12 | 100 | 60.6 | 15 | 40.5 | 59 |
| | Interval debulking | 0 | 0 | NA | 22 | 59.5 | 64.5 |
| Stage: | Early stage (STIC-I-II) | 3 | 25 | 57 | 1 | 2.7 | 48 |
| | Late stage (III-IV) | 9 | 75 | 61.8 | 36 | 97.3 | 62.3 |
| BRCA status: | Germline mutation | 0 | 0 | NA | 10 | 24.3 | 54.1 |
| | No mutation | 6 | 50 | 58.5 | 12 | 35.1 | 63.3 |
| | Unknown | 6 | 50 | 62.7 | 15 | 40.5 | 66.9 |
| Control cohort: | | 12 | 100 | 54.2 | 115 | 100 | 50.1 |
| Indication for | Benign ovarian mass | 6 | 50 | 46 | 28 | 23.9 | 54.8 |
| surgery: | | | | | | | |
| | Endometrial polyp | 3 | 25 | 61.7 | 9 | 7.7 | 61.7 |
| | Menometrorrhagia | 0 | 0 | NA | 12 | 10.3 | 49.8 |
| | Uterine prolapse | 1 | 8 | 74 | 14 | 12 | 62.4 |
| | Leio-mymatous uterus | 0 | 0 | NA | 10 | 8.5 | 45.2 |
| | Risk reducing BSO | 0 | 0 | NA | 20 | 17.1 | 46.8 |
| | Gestational residua | 0 | 0 | NA | 10 | 8.5 | 30.8 |
| | Normal Endometrium | 2 | 6.8 | 58 | 6 | 5.1 | 50.8 |
| | Other | 0 | 0 | NA | 8 | 6.8 | 36.9 |
| High Risk Cohort: | | | | NA | 25 | 100 | 32.7 |

TABLE 2
Clinical data of UtL samples.

| # | Set | Sample ID | Diagnosis | Age | NACT | BRCA Status |
|---|---|---|---|---|---|---|
| 1 | Discovery | MUL-22 | HGOC | 66 | NO | ND |
| 2 | Discovery | MUL-33 | HGOC | 67 | NO | ND |
| 3 | Discovery | MUL-61 | HGOC | 63 | NO | ND |
| 4 | Discovery | UL-21 | HGOC | 48 | NO | ND |
| 5 | Discovery | UL-34 | HGOC | 52 | NO | ND |
| 6 | Discovery | UL-6 | HGOC | 55 | NO | ND |
| 7 | Discovery | BUL-47 | HGOC | 63 | NO | NK |
| 8 | Discovery | BUL-48 | HGOC | 64 | NO | NK |
| 9 | Discovery | BUL-5 | HGOC | 56 | NO | NK |
| 10 | Discovery | BUL-82 | HGOC | 49 | NO | NK |
| 11 | Discovery | BUL-90 | HGOC | 66 | NO | NK |
| 12 | Discovery | UL-51 | HGOC | 78 | NO | NK |
| 13 | Discovery | BUL-103 | Benign ovarian mass | 51 | | |
| 14 | Discovery | BUL-30 | Benign ovarian mass | 56 | | |
| 15 | Discovery | BUL-91 | Benign ovarian mass | 62 | | |
| 16 | Discovery | UL-1 | Benign ovarian mass | 35 | | |
| 17 | Discovery | UL-4 | Benign ovarian mass | 49 | | |
| 18 | Discovery | UL-50 | Benign ovarian mass | 23 | | |
| 19 | Discovery | MUL-11 | Endometrial polyp | 61 | | |
| 20 | Discovery | MUL-54 | Endometrial polyp | 57 | | |
| 21 | Discovery | UL-15 | Endometrial polyp | 67 | | |
| 22 | Discovery | MUL-12 | Normal endometrium | 49 | | |
| 23 | Discovery | MUL-78 | Normal endometrium | 67 | | |
| 24 | Discovery | BUL-40 | Uterine prolapse | 74 | | |
| 25 | Validation | UL-25 | HGOC | 38 | NO | BRCA |
| 26 | Validation | UL-11 | HGOC | 64 | NO | BRCA1 |
| 27 | Validation | UL-20 | HGOC | 73 | NO | BRCA1 |
| 28 | Validation | UL-37 | HGOC | 42 | NO | BRCA1 |
| 29 | Validation | UL-14 | HGOC | 59 | NO | BRCA2 |
| 30 | Validation | UL-40 | HGOC | 47 | NO | BRCA2 |
| 31 | Validation | UL-41 | HGOC | 54 | NO | BRCA2 |
| 32 | Validation | MUL-60 | HGOC | 63 | NO | ND |
| 33 | Validation | MUL-92 | HGOC | 78 | NO | ND |
| 34 | Validation | UL-39 | HGOC | 60 | NO | ND/Family Hx |
| 35 | Validation | BUL-127 | HGOC | 48 | NO | NK |
| 36 | Validation | BUL-9 | HGOC | 49 | NO | NK |
| 37 | Validation | MUL-81 | HGOC | 68 | NO | NK |
| 38 | Validation | MUL-86 | HGOC | 63 | NO | NK |
| 39 | Validation | UL-45 | HGOC | 79 | NO | NK |
| 40 | Validation | BUL-2 | HGOC | 50 | YES | BRCA1 |
| 41 | Validation | MUL-21 | HGOC | 48 | YES | BRCA1 |
| 42 | Validation | UL-28 | HGOC | 66 | YES | BRCA1 |
| 43 | Validation | BUL-19 | HGOC | 67 | YES | ND |
| 44 | Validation | BUL-4 | HGOC | 70 | YES | ND |
| 45 | Validation | BUL-51 | HGOC | 70 | YES | ND |
| 46 | Validation | BUL-63 | HGOC | 72 | YES | ND |
| 47 | Validation | MUL-4 | HGOC | 81 | YES | ND |
| 48 | Validation | MUL-40 | HGOC | 66 | YES | ND |
| 49 | Validation | UL-29 | HGOC | 76 | YES | ND |
| 50 | Validation | UL-33 | HGOC | 48 | YES | ND |
| 51 | Validation | UL-36 | HGOC | 57 | YES | ND |
| 52 | Validation | UL-5 | HGOC | 70 | YES | ND |
| 53 | Validation | UL-7 | HGOC | 69 | YES | ND |
| 54 | Validation | MUL-62 | HGOC | 57 | YES | ND/Family Hx |
| 55 | Validation | BUL-118 | HGOC | 60 | YES | NK |
| 56 | Validation | BUL-130 | HGOC | 62 | YES | NK |
| 57 | Validation | BUL-15 | HGOC | 65 | YES | NK |
| 58 | Validation | BUL-25 | HGOC | 67 | YES | NK |
| 59 | Validation | BUL-6 | HGOC | 74 | YES | NK |
| 60 | Validation | UL-30 | HGOC | 60 | YES | NK |
| 61 | Validation | UL-32 | HGOC | 64 | YES | NK |
| 62 | Validation | UL-46 | Benign ovarian mass | 65 | | BRCA2 |
| 63 | Validation | BUL-102 | Benign ovarian mass | 54 | | |
| 64 | Validation | BUL-11 | Benign ovarian mass | 61 | | |
| 65 | Validation | BUL-111 | Benign ovarian mass | 38 | | |
| 66 | Validation | BUL-119 | Benign ovarian mass | 66 | | |
| 67 | Validation | BUL-122 | Benign ovarian mass | 23 | | |
| 68 | Validation | BUL-124 | Benign ovarian mass | 70 | | |
| 69 | Validation | BUL-125 | Benign ovarian mass | 83 | | |
| 70 | Validation | BUL-17 | Benign ovarian mass | 81 | | |
| 71 | Validation | BUL-23 | Benign ovarian mass | 42 | | |
| 72 | Validation | BUL-26 | Benign ovarian mass | 66 | | |
| 73 | Validation | BUL-28 | Benign ovarian mass | 60 | | |
| 74 | Validation | BUL-35 | Benign ovarian mass | 30 | | |
| 75 | Validation | BUL-41 | Benign ovarian mass | 66 | | |
| 76 | Validation | BUL-49 | Benign ovarian mass | 42 | | |
| 77 | Validation | BUL-50 | Benign ovarian mass | 27 | | |
| 78 | Validation | BUL-53 | Benign ovarian mass | 30 | | |
| 79 | Validation | BUL-54 | Benign ovarian mass | 73 | | |
| 80 | Validation | BUL-69 | Benign ovarian mass | 33 | | |
| 81 | Validation | BUL-75 | Benign ovarian mass | 62 | | |
| 82 | Validation | BUL-81 | Benign ovarian mass | 64 | | |
| 83 | Validation | BUL-92 | Benign ovarian mass | 70 | | |
| 84 | Validation | MUL-68 | Benign ovarian mass | 49 | | |
| 85 | Validation | MUL-90 | Benign ovarian mass | 70 | | |
| 86 | Validation | UL-42 | Benign ovarian mass | 52 | | |
| 87 | Validation | UL-44 | Benign ovarian mass | 41 | | |
| 88 | Validation | UL-47 | Benign ovarian mass | 70 | | |
| 89 | Validation | UL-53 | Benign ovarian mass | 46 | | |
| 90 | Validation | BUL-38 | Chronic pelvic pain | 40 | | |
| 91 | Validation | BUL-60 | Elongation of cervix | 59 | | |
| 92 | Validation | BUL-84 | Endometrial polyp | 64 | | |
| 93 | Validation | MUL-19 | Endometrial polyp | 59 | | |
| 94 | Validation | MUL-24 | Endometrial polyp | 67 | | |
| 95 | Validation | MUL-25 | Endometrial polyp | 68 | | |
| 96 | Validation | MUL-27 | Endometrial polyp | 46 | | |
| 97 | Validation | MUL-34 | Endometrial polyp | 64 | | |
| 98 | Validation | MUL-36 | Endometrial polyp | 63 | | |
| 99 | Validation | MUL-77 | Endometrial polyp | 72 | | |
| 100 | Validation | MUL-9 | Endometrial polyp | 52 | | |
| 101 | Validation | BUL-73 | Endometriosis | 41 | | |
| 102 | Validation | MUL-29 | Gestational residua | 25 | | |
| 103 | Validation | MUL-37 | Gestational residua | 33 | | |
| 104 | Validation | MUL-5 | Gestational residua | 22 | | |
| 105 | Validation | MUL-87 | Gestational residua | 41 | | |
| 106 | Validation | MUL-88 | Gestational residua | 33 | | |
| 107 | Validation | UL-12 | Gestational residua | 35 | | |
| 108 | Validation | UL-18 | Gestational residua | 38 | | |
| 109 | Validation | UL-26 | Gestational residua | 24 | | |
| 110 | Validation | UL-8 | Gestational residua | 21 | | |
| 111 | Validation | UL-9 | Gestational residua | 36 | | |
| 112 | Validation | BUL-39 | Hydrosalpinx | 32 | | |
| 113 | Validation | BUL-12 | Leiomyomatous uterus | 48 | | |
| 114 | Validation | BUL-135 | Leiomyomatous uterus | 50 | | |
| 115 | Validation | BUL-16 | Leiomyomatous uterus | 53 | | |
| 116 | Validation | BUL-52 | Leiomyomatous uterus | 46 | | |
| 117 | Validation | BUL-64 | Leiomyomatous uterus | 49 | | |
| 118 | Validation | BUL-80 | Leiomyomatous uterus | 49 | | |
| 119 | Validation | MUL-26 | Leiomyomatous uterus | 47 | | |
| 120 | Validation | MUL-76 | Leiomyomatous uterus | 29 | | |
| 121 | Validation | UL-16 | Leiomyomatous uterus | 43 | | |
| 122 | Validation | UL-17 | Leiomyomatous uterus | 38 | | |
| 123 | Validation | BUL-24 | Mechanical infertility | 39 | | |
| 124 | Validation | MUL-10 | Mechanical infertility | 33 | | |
| 125 | Validation | BUL-1 | Menometrorrhagia | 55 | | |
| 126 | Validation | BUL-10 | Menometrorrhagia | 44 | | |
| 127 | Validation | BUL-20 | Menometrorrhagia | 54 | | |
| 128 | Validation | BUL-22 | Menometrorrhagia | 57 | | |
| 129 | Validation | BUL-27 | Menometrorrhagia | 48 | | |
| 130 | Validation | BUL-33 | Menometrorrhagia | 50 | | |
| 131 | Validation | BUL-94 | Menometrorrhagia | 42 | | |
| 132 | Validation | MUL-1 | Menometrorrhagia | 50 | | |
| 133 | Validation | UL-10 | Menometrorrhagia | 54 | | |
| 134 | Validation | UL-13 | Menometrorrhagia | 44 | | |
| 135 | Validation | UL-2 | Menometrorrhagia | 51 | | |
| 136 | Validation | MUL-2 | Normal endometrium | 37 | | |
| 137 | Validation | MUL-3 | Normal endometrium | 36 | | |
| 138 | Validation | MUL-35 | Normal endometrium | 48 | | |
| 139 | Validation | MUL-91 | Normal endometrium | 78 | | |
| 140 | Validation | UL-43 | Normal endometrium | 49 | | |
| 141 | Validation | BUL-13 | Pelvic inflammatory disease | 27 | | |
| 142 | Validation | BUL-85 | Pelvic inflammatory disease | 24 | | |
| 143 | Validation | BUL-3 | RRBSO | 34 | | BRCA |
| 144 | Validation | BUL-56 | RRBSO | 38 | | BRCA |
| 145 | Validation | BUL-57 | RRBSO | 48 | | BRCA |
| 146 | Validation | MUL-30 | RRBSO | 46 | | BRCA |
| 147 | Validation | MUL-8 | RRBSO | 41 | | BRCA |
| 148 | Validation | BUL-121 | RRBSO | 44 | | BRCA1 |
| 149 | Validation | BUL-131 | RRBSO | 46 | | BRCA1 |
| 150 | Validation | BUL-134 | RRBSO | 39 | | BRCA1 |
| 151 | Validation | BUL-14 | RRBSO | 53 | | BRCA1 |
| 152 | Validation | BUL-55 | RRBSO | 37 | | BRCA1 |
| 153 | Validation | BUL-96 | RRBSO | 45 | | BRCA1 |
| 154 | Validation | MUL-95 | RRBSO | 56 | | BRCA1 |
| 155 | Validation | UL-48 | RRBSO | 56 | | BRCA1 + BRCA2 |
| 156 | Validation | BUL-112 | RRBSO | 40 | | BRCA2 |
| 157 | Validation | BUL-42 | RRBSO | 53 | | BRCA2 |
| 158 | Validation | BUL-72 | RRBSO | 54 | | BRCA2 |
| 159 | Validation | BUL-88 | RRBSO | 47 | | BRCA2 |
| 160 | Validation | BUL-78 | RRBSO | 50 | | ND |
| 161 | Validation | BUL-21 | RRBSO | 60 | | ND/Family Hx |
| 162 | Validation | BUL-8 | RRBSO | 50 | | BRCA |
| 163 | Validation | BUL-18 | Uterine prolapse | 72 | | |
| 164 | Validation | BUL-29 | Uterine prolapse | 74 | | |
| 165 | Validation | BUL-32 | Uterine prolapse | 70 | | |
| 166 | Validation | BUL-34 | Uterine prolapse | 63 | | |
| 167 | Validation | BUL-36 | Uterine prolapse | 57 | | |
| 168 | Validation | BUL-43 | Uterine prolapse | 62 | | |
| 169 | Validation | BUL-44 | Uterine prolapse | 70 | | |
| 170 | Validation | BUL-58 | Uterine prolapse | 69 | | |
| 171 | Validation | BUL-65 | Uterine prolapse | 64 | | |
| 172 | Validation | BUL-7 | Uterine prolapse | 56 | | |
| 173 | Validation | BUL-76 | Uterine prolapse | 50 | | |
| 174 | Validation | BUL-86 | Uterine prolapse | 49 | | |
| 175 | Validation | BUL-87 | Uterine prolapse | 48 | | |
| 176 | Validation | BUL-93 | Uterine prolapse | 70 | | |
| 177 | High Risk | ULBRCA-10 | High risk FU | 29 | | BRCA1 |
| 178 | High Risk | ULBRCA-10a | High risk FU | 30 | | BRCA1 |
| 179 | High Risk | ULBRCA-12 | High risk FU | 34 | | BRCA1 |
| 180 | High Risk | ULBRCA-14 | High risk FU | 36 | | BRCA1 |
| 181 | High Risk | ULBRCA-15 | High risk FU | 30 | | BRCA1 |
| 182 | High Risk | ULBRCA-17 | High risk FU | 33 | | BRCA1 |
| 183 | High Risk | ULBRCA-18 | High risk FU | 32 | | BRCA1 |
| 184 | High Risk | ULBRCA-19 | High risk FU | 39 | | BRCA1 |
| 185 | High Risk | ULBRCA-2 | High risk FU | 31 | | BRCA1 |
| 186 | High Risk | ULBRCA-20 | High risk FU | 36 | | BRCA1 |
| 187 | High Risk | ULBRCA-21 | High risk FU | 40 | | BRCA1 |
| 188 | High Risk | ULBRCA-22 | High risk FU | 34 | | BRCA1 |
| 189 | High Risk | ULBRCA-3 | High risk FU | 32 | | BRCA1 |
| 190 | High Risk | ULBRCA-3a | High risk FU | 33 | | BRCA1 |
| 191 | High Risk | ULBRCA-4 | High risk FU | 33 | | BRCA1 |
| 192 | High Risk | ULBRCA-5 | High risk FU | 25 | | BRCA1 |
| 193 | High Risk | ULBRCA-5a | High risk FU | 26 | | BRCA1 |
| 194 | High Risk | ULBRCA-8 | High risk FU | 38 | | BRCA1 |
| 195 | High Risk | ULBRCA-9 | High risk FU | 32 | | BRCA1 |
| 196 | High Risk | ULBRCA-1 | High risk FU | 38 | | BRCA2 |
| 197 | High Risk | ULBRCA-11 | High risk FU | 34 | | BRCA2 |
| 198 | High Risk | ULBRCA-13 | High risk FU | 28 | | BRCA2 |
| 199 | High Risk | ULBRCA-16 | High risk FU | 30 | | BRCA2 |
| 200 | High Risk | ULBRCA-1a | High risk FU | 39 | | BRCA2 |
| 201 | High Risk | ULBRCA-6 | High risk FU | 27 | | BRCA2 |
| 202 | Excluded | BUL-109 | Borderline tumor | 64 | | |
| 203 | Excluded | UL-19 | Borderline tumor | 30 | | |
| 204 | Excluded | UL-22 | Borderline tumor | 77 | | |
| 205 | Excluded | UL-3 | Borderline tumor | 26 | | |
| 206 | Excluded | UL-23 | Endometrial carcinoma | 68 | | 206 |
| 207 | Excluded | BUL-101 | Granulosa cell tumor | 45 | | 207 |
| 208 | Excluded | UL-27 | Menometrorrhagia | 49 | | 208 |
| 209 | Excluded | UL-35 | Mucinous adenocarcinoma | 54 | | 209 |
| | | | of appendix | | | |
| 210 | Excluded | MUL-38 | No clinical information | ? | | 210 |
| 211 | Excluded | MUL-44 | Normal endometrium | 57 | | 211 |
| 212 | Excluded | MUL-69 | Undifferentiated sarcoma of | 58 | | 212 |
| | | | ovary | | | |

NACT—neoadjuvant chemotherapy (in case of HGOC Tumors); BRCA status (for RRBSO and HGOC Tumors) designated as BRCA for carriers, ND—no mutation detected, NK—unknown; stage determined according to FIGO staging system.

Lavage Collection Technique

Uterine lavage samples were collected before surgery, after induction of anesthesia by gynecologists.

An intrauterine insemination catheter (Insemi™-Cath, Cook Inc. Bloomington, Ind., USA) or rigid pipelle uterine sampler (Endosampler, MedGyn, Addison, Ill., USA) was inserted into the endometrial space through the cervical canal. 10 mL of saline were flushed into the uterine cavity and fallopian tubes and immediately retrieved; some backflow was often observed and fluid pooling in the vaginal speculum was also aspirated. A total of 212 samples at an average volume of 4.6 mL were collected.

Sample Preparation

The UtL samples were centrifuged at 480×g to eliminate cells. Supernatants were aliquoted within 6 hours from the procedure. UtL aliquots and cell pellets were kept in −80° c until use. Microvesicle isolation was performed according to the protocol developed herein [17]. Briefly, UtLF samples were centrifuged at 1000×g to remove cell debris, followed by microvesicle precipitation by centrifugation at 20,000×g for 60 min at 4° C. Pellet was then washed with 1 ml ice-cold PBS and centrifuged again at 20,000×g for 60 min at 4° C.

Primary fresh frozen HGOC tumors were obtained from the Chaim Sheba Institutional Tumor Bank. H&E staining was performed to ensure >80% tumor cells in the section. The frozen tissue was then homogenized for RNA extraction.

Fresh benign FT fimbriae were obtained from the Chaim Sheba Institutional Tumor Bank. Tissues were allocated from women with gynecological conditions not affecting the FT, after gross pathological examination. The fimbriae were processed as previously described [17], [18]. Briefly, fimbriae were incubated in dissociation medium (DMEM, Biological Industries, Israel) supplemented with 1.4 mg/ml Pronase (Roche Applied Science, Indianapolis, Ind., USA) and 0.1 mg/ml DNase (Sigma-Aldrich, St. Louis, Mo., USA) for 48 hours at 4° C. with constant mild agitation. The dissociated epithelial cells were harvested by centrifugation and were kept as cell pellet in −80° c until use.

Microvesicle Proteomics

Microvesicle pellets were solubilized in 8M urea in 100 mM Tris-HCl (pH 8.5), followed by overnight in-solution trypsin digestion. Resulting peptides were purified on C₁₈ StageTips (3M Empore™, St. Paul, Minn., USA). Peptides were analyzed by liquid-chromatography using the EASY-nLC1000 HPLC coupled to high resolution mass spectrometric analysis on the Q-Exactive Plus or Q-Exactive HF mass spectrometers (ThermoFisher Scientific, Waltham, Mass., USA). Peptides were separated on 50 cm EASY-spray columns (ThermoFisher Scientific) with a 240 min gradient. MS acquisition was performed in a data-dependent mode with selection of the top 10 peptides from each MS spectrum for fragmentation and MS/MS analysis. Raw MS files were analyzed in the MaxQuant software and the Andromeda search engine (Cox J, et al. Nat Biotechnol 26:1367-1372 (2008); Cox J, et al: J Proteome Res 10:1794-1805, (2011)). Database search was performed using the Uniprot database, and included carbamidomethyl-cysteine as a fixed modification, and N-terminal acetylation and methionine oxidation as a variable modification. A reverse decoy database was used to determine false discovery rate of 1% on the peptide and protein level. The label-free algorithm in MaxQuant was used to retrieve the quantitative information.

Computational Analysis

All the statistical analyses were performed with the Perseus program ((Tyanova S, et al., Nat Methods (2016)). The data was filtered to include proteins with valid values in at least 80% of the samples. Missing values were then imputed with random values that form a normal distribution with a width of 30% and downshift of 1.8 standard deviations of the general data distribution.

The samples were divided into discovery (n=24) and validation cohorts (n=152). Classifier optimization was performed using support vector machines (SVM) for classification, and three feature selection algorithms: recursive feature elimination (RFE) -SVM (RFE-SVM)-based, SVM and ANOVA (32). Cross validation was performed by 250 iterations of random sampling of 85% of the samples as test and 15% as validation. The optimal number of overlapping features of these three analytic methods was calculated to provide highest predictive accuracy. The performance of the extracted classifier was then blindly examined on the validation cohort.

RNA Extraction and qRT-PCR

Fresh-frozen HGOC tumors and fresh grossly benign FT fimbriae were obtained from the Chaim Sheba Institutional Tumor Bank. H&E staining was performed to ensure >80% tumor cellularity. The fimbriae were processed as previously described [14-15]. Total RNA was extracted from primary fresh frozen HGOC tumors and dissociated normal FTE cells using QIAzol reagent (Qiagen, Valencia, Calif., USA) followed by RNeasy clean-up kit (Qiagen) according to manufacturer's protocol. Gene expression was assessed using FastStart Universal SYBR Green Master (ROX) (Roche). Primers for the signature-genes are listed in Table 3 (Sigma-Aldrich).

TABLE 3
Primers used for RT-PCR evaluation the signature-genes expression

| | Genebank | Primer sense | Primer antisense | |
|---|---|---|---|---|
| | accession | (5′-3′) | (5′-3′) | Amplicon |
| Gene name | number | SEQ ID NO: | SEQ ID NO: | size |
| OVGP1 | NM_002557 | TATGTCCCGTATGCCAACAA | TCCATGTCCAATGTCCACAC | 128 |
| | | SEQ ID NO: 57 | SEQ ID NO: 58 | |
| S100A14 | NM_020672 | AGCGGCTGCCAACAGATCA | ACTGTGTCTGGTCCTTTGGTG | 86 |
| | | SEQ ID NO: 59 | SEQ ID NO: 60 | |
| SERPINB5 | NM_002639 | CATGTTCATCCTACTACCCAAGG | TCTGAGTTGAGTTGTTTTTCAATCTT | 78 |
| | | SEQ ID NO: 61 | SEQ ID NO: 62 | |
| SPRR3b | NM_005416 | ACCAGAGCCATGTCCTTCAA | ATCTGGTGGTTGGCTTCTCA | 105 |
| | | SEQ ID NO: 63 | SEQ ID NO: 64 | |
| ENPP3 | NM_005021 | TGTCACGGGCTTGTATCCAG | TGCCACCAGGCTGGATTATT | 117 |
| | | SEQ ID NO: 65 | SEQ ID NO: 66 | |
| CLUAP1 | NM_001330454 | CCAAGCCACAGACAGCCAT | TCTCCACCTTGCATCGTGC | 79 |
| | | SEQ ID NO: 67 | SEQ ID NO: 68 | |
| CLCA4 | NM_012128/ | TCACTTCACCCCTGACCTTC | GAGCCCACTCATGGACAAAC | 83 |
| | NR_024602 | SEQ ID NO: 69 | SEQ ID NO: 70 | |
| CEACAM5 | NM_001308398 | CAATAGGACCACAGTCACGACG | GGTTGGAGTTGTTGCTGGTGAT | 77 |
| | | AT | SEQ ID NO: 72 | |
| | | SEQ ID NO: 71 | | |
| RNASE3 | NM_002935 | CAGAGACTGGGAAACATGGT | AACCACTGAGCCCTCGTAAA | 128 |
| | | SEQ ID NO: 73 | SEQ ID NO: 74 | |

Immunohistochemistry (IHC)

Archival tissues were retrieved from the Department of Pathology at the Chaim Sheba Medical Center with the appropriate ethical committee approvals. Tissue microarrays (TMAs) of ˜30 representative cases (in duplicates) were constructed of morphologically benign fimbriae of patients with the following diagnoses: (i) normal FT adjacent to HGOC (median age=60, range: 40-74), (ii) tubal ectopic pregnancy (EP, median age=33, range: 20-45), (iii) leiomyomatous uterus (LM, median age=52, range: 38-67) and (iv) RRBSO (median age=43, range: 35-66). TMA of 46 HGOC tumors (median age=62, range: 30-88) was also constructed. All slides were simultaneously stained and scored for staining intensity and distribution, on a scale of 0-3 (0—no staining or faint staining in <10% of cells, 1—faint staining in >10% of cells, 2—moderate staining of >10% of cells, and 3—strong staining of >10% of cells). Primary antibodies used: (i) anti-OVGP1 (HPA062205, 1:50, positive control: FTE), (ii) anti-SERPINB5 (HPA020136, 1:200, positive control: keratinocytes) and (iii) anti-S100A14 (HPA027613, 1:1000, positive control: keratinocytes) (Sigma-Aldrich, St. Louis, Mo., USA).

Statistical Analysis

Statistical significance (p<0.05) was assessed by Student t-test for RT-PCR data or by Fisher exact test for IHC intensity scores. Multivariate correlation analysis was used to exclude age and menopausal status confounders.

### Example 1

Patients' Characteristics

Aiming to identify early-stage biomarkers for HGOC, it was hypothesized that “localized liquid biopsy” such as UtL sampling is likely to have better sensitivity and specificity than serum biomarkers. To that end, a set of 212 UtL samples from 208 enrolled donors was analyzed (Tables 1 and 2). Eleven samples were excluded due to missing data (n=1), inappropriate ovarian tumor histological subtype (n=8), or failing the quality control measures (n=2). The discovery set (n=24) consisted of UtL samples from 12 HGOC patients and 12 representative controls from all participating medical centers, while all subsequent samples were regarded as a validation set (n=152), and analyzed independently in a blinded manner. Overall, 49 UtL samples were obtained from HGOC patients (patient cohort', average age=61.8). Of those, 27 samples were obtained at primary debulking surgery and the other 22 were obtained at interval debulking surgery, after 3 cycles of platinum/taxane neoadjuvant chemotherapy. Forty five patients (91.8%) were diagnosed with stage III-IV disease, and 4 were obtained from patients with stage IA-II disease. All patients were appropriately staged according to International Federation of Obstetrics and Gynecology (FIGO) guidelines. One case of an occult serous tubal intraepithelial carcinoma (STIC) incidentally detected following RRBSO surgery was also included. The control cohort included 127 UtL samples of patients undergoing gynecological surgical procedures for non-malignant indications (average age=50.5). Eligible diagnoses included: benign ovarian masses or cysts, endometrial polyp, uterine prolapse, menometrorrhagia, gestational residua (post-abortion or post-partum), leiomyomatous uterus, RRBSO due to BRCA germline mutation or family history, and other benign gynecologic conditions. In addition, 25 UtL liquid biopsies from 21 healthy BRCA1/2 mutation carriers (average age=32.7), who did not yet undergo RRBSO were analyzed. Additional clinical characteristics of the discovery and validation sets are outlined in Table 1 and Table 2.

### Example 2

UtL microvesicle Proteomic Profiling

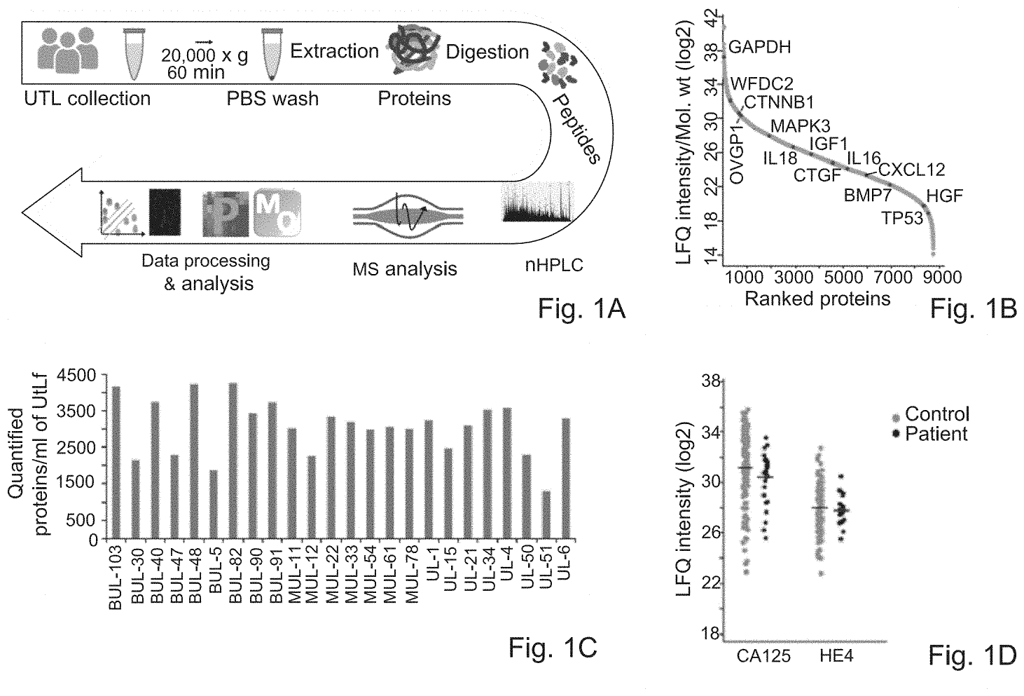

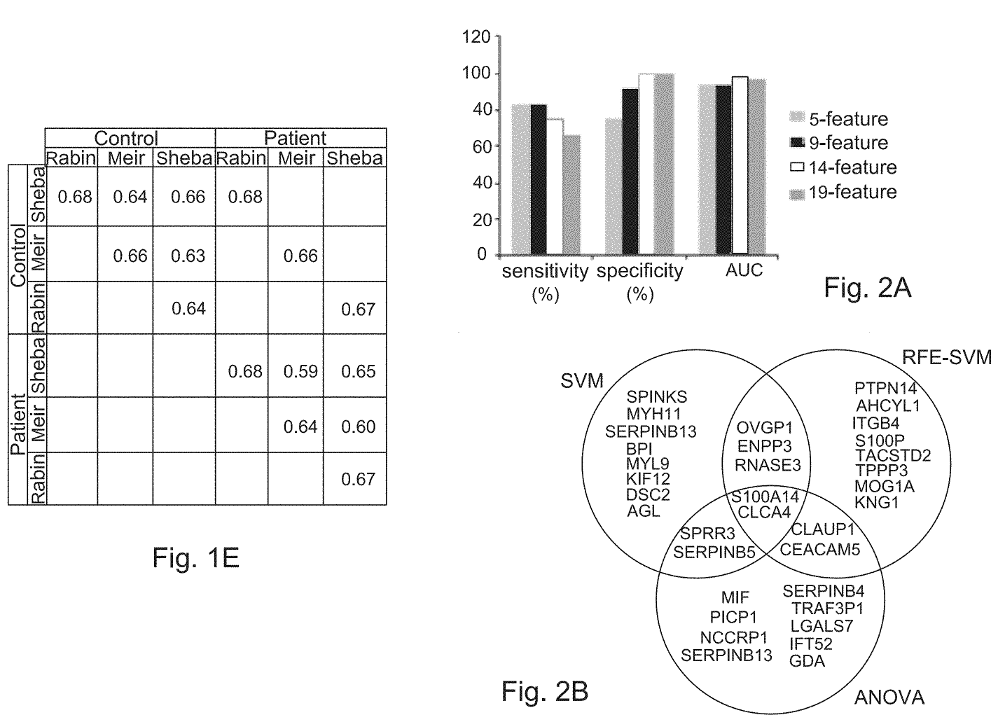

In order to profile the proteome of a complex body fluid and detect potential diagnostic biomarkers, the challenge inflicted by the existence of highly abundant proteins had to be overcome. Therefore the previously developed method for microparticle isolation from plasma was examined for the application to UtL samples. Therefore, microvesicles were isolated from UtL by high speed centrifugation followed by PBS wash to remove albumin contamination. The microvesicles and their protein content were denatured with urea, followed by trypsin protein digestion and LC-MS/MS analysis as illustrated by the scheme of FIG. 1A. Analysis of the entire discovery cohort identified a total of 8578 UtLF microvesicle proteins and an average number 3000 per sample (range: 1500-4000) (FIG. 1C). Among the identified proteins, known FTE/HGOC proteins were found, such as MUC16 (CA125), WFDC2 (HE4), and OVGP1 (MUC9), as well as lower abundance proteins, including cytokines and growth factors, such as IGF1, CXCL12, IL18 and HGF (FIG. 1B). The dynamic range of relative abundance of the microvesicle proteome spans 8 orders of magnitude. In agreement with previous results of the inventors [18], the amounts of mullerian tract lineage markers such as CA125 (MUC16) and HE4 (WFDC2) as measured by MS, did not discriminate between HGOC patients and control samples. (FIG. 1D). These results further emphasize the urgent need for better markers that reflect early disease state rather than the normal tissue markers. Moreover, the concentration of CA125 in unfractionated UtL was measured with a commercial assay (Access Immunoassay OV Monitor, Beckman Coulter), and demonstrated no significant difference between patients and controls (data not shown). Since the samples were collected in three medical centers, potential ‘batch effect’ or differences in composition of samples were excluded (surrogate for UtL sampling technique variations). Correlation analysis between samples showed an average correlation of 0.67 within each center and correlation of 0.66 between centers. Furthermore, higher correlations were found between controls from different centers, than between patients and controls from the same center (FIG. 1E). It was therefore concluded that the batch effects and inter-institutional differences are negligible.

### Example 3

Identification of Protein Signature

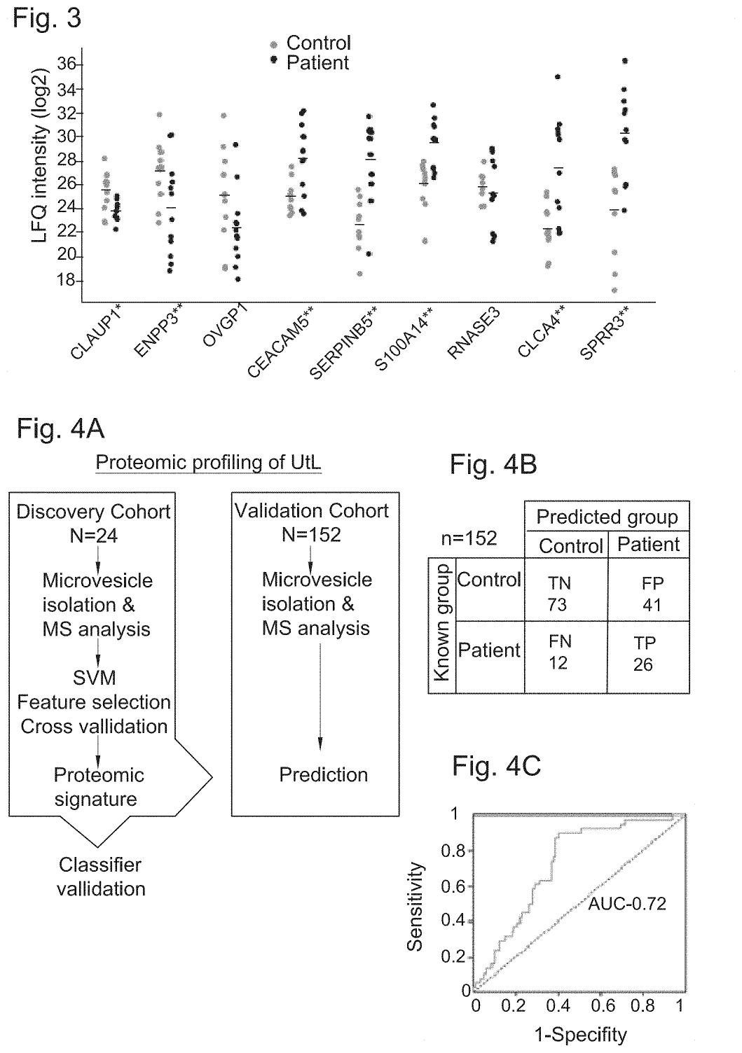

Next, the proteomic profiles of 24 patients and controls (discovery cohort') were used to construct a protein classifier for HGOC diagnosis. Support vector machine algorithm was used to classify the samples, and optimized the minimal number of features (proteins) that provide highest accuracy. For feature selection, 3 different algorithms were applied to the discovery cohort MS-datasets, SVM, RFE-SVM and ANOVA. The entire analytical workflow was embedded in a cross validation procedure to reduce over-fitting in order to identify a signature with a minimal number of proteins, a high predictive power, and a least dependence on the feature selection algorithm. The performance of several sets of top-ranked overlapping proteins, ranging in size from 5 to 19 features (FIG. 2A, 2B) was therefore examined. Optimal sensitivity, specificity, and area under the curve (AUC) of Receiver Operating Characteristic (ROC) curve of sensitivity vs. 1-specificity were obtained with a 9-protein signature, 6 of which were higher in the HGOC patients, and 3 that were higher in controls (FIG. 2C, FIG. 3, and Table 4). Overall, this signature demonstrated 83% sensitivity and 91.6% specificity and an AUC of 0.94 in the discovery set (FIG. 2D). Importantly, this signature correctly predicted all 3 stage IA HGOC cases included in the discovery set.

TABLE 4
The overlapping features which compose the 9-protein classifier.

| | | UNIPROT | SVM | RFE-SVM | ANOVA |
|---|---|---|---|---|---|
| Gene names | Protein names | ID | rank | rank | rank |
| OVGP1 | Oviduct-specific glycoprotein | Q12889 | 1 | 6 | 208 |
| SPRR3 | Small proline-rich protein 3 | Q9UBC9 | 2 | 33 | 10 |
| CLCA4 | Calcium-activated chloride channel | Q14CN2 | 3 | 3 | 5 |
| | regulator 4 | | | | |
| S100A14 | Protein S100-A14 | Q9HCY8 | 14 | 8 | 2 |
| CLUAP1 | Clusterin-associated protein 1 | Q96AJ1 | 44 | 10 | 7 |
| SERPINB5 | Serpin B5 | P36952 | 11 | 4 | 4 |
| RNASE3 | Eosinophil cationic protein | P12724 | 12 | 1 | 1746 |
| CEACAM5 | Carcinoembryonic antigen-related cell | P06731 | 33 | 13 | 6 |
| | adhesion molecule 5 | | | | |
| ENPP3 | Ectonucleotidepyrophosphatase/ | O14638 | 7 | 15 | 93 |
| | phosphodiesterase family member 3 | | | | |
| CEACAM5 | Carcinoembryonic antigen-related cell | P06731 | 33 | 13 | 6 |
| | adhesion molecule 5 | | | | |
| ENPP3 | Ectonucleotidepyrophosphatase/ | O14638 | 7 | 15 | 93 |
| | phosphodiesterase family member 3 | | | | |

Following, the 9 biomarker proteins described herein were ranked in order of importance as provided in Table 5.

TABLE 5
The 9-signature proteins ranked by significance

| Protein | Rank |
|---|---|
| CLCA4 | 1 |
| OVGP1 | 2 |
| S100A14 | 3 |
| SPRR3 | 4 |
| RNASE3 | 5 |
| SERPINB5 | 6 |
| CLUAP1 | 7 |
| CEACAM5 | 8 |
| ENPP3 | 9 |

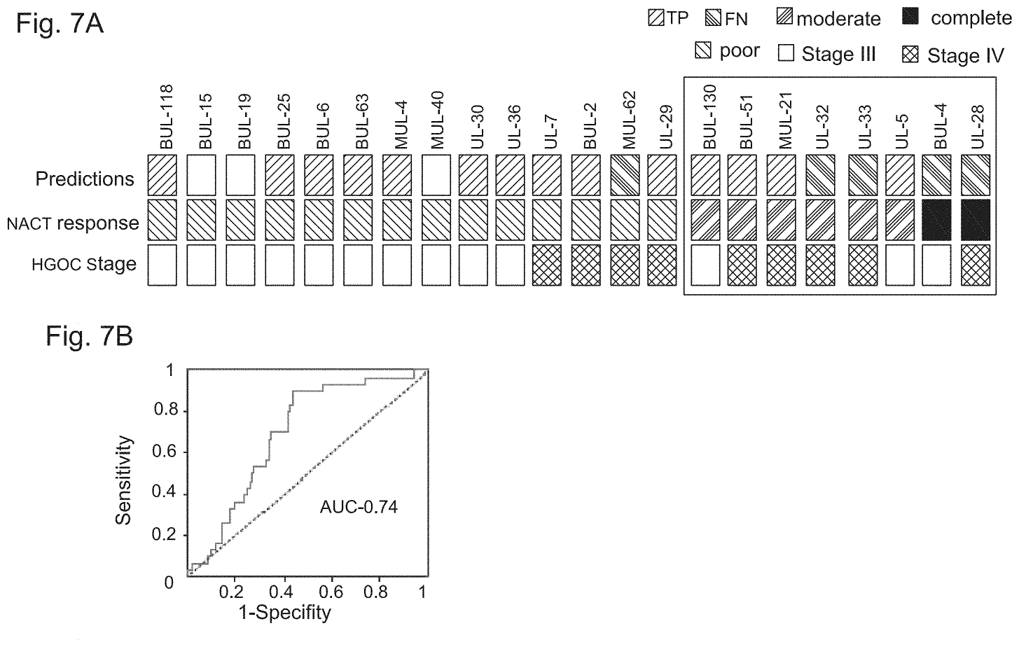

In order to validate the performance of the proteomic signature on an independent patient/control UtL sample set (validation cohort', n=152, FIG. 4A), an unbiased, blinded, microvesicle proteomic profiling was performed as described above, and identified a total of 8760 proteins, and an average of 3200 per sample. Application of the 9-protein classifier to the validation cohort correctly predicted 73 of the controls correctly (Specificity=64% and NPV=85.9%) and 26 patients correctly (Sensitivity=68.4% and PPV=38.8%) (FIG. 4B-4C). ROC curve for the validation cohort showed an AUC of 0.72. Of note, one case of an incidental occult STIC was correctly designated as ‘tumor’ by the 9-protein classifier. Looking specifically at the 4 early-stage samples, the 9-proteins signature better discriminated them from control samples than it did for advanced stage HGOC samples (FIG. 5), suggesting a trend towards better identification of early-stage lesions. However, due to the small number of early stage patients, these differences require further investigation. Looking at the entire cohort, the classifier offered 71.4% sensitivity and 59% specificity (PPV=36.5% NPV=86.6%) for diagnosis of HGOC. The validation set included 22 UtL samples from HGOC patients who received neo-adjuvant chemotherapy (NACT). Overall, the NACT treated samples were highly similar to the samples obtained from HGOC patients during primary debulking (FIG. 6), and eight of these cases were falsely predicted as “Normal”. To examine the association between the prediction accuracy and the response to therapy, the quality of response to NACT was scored in each case based on pathological and imaging reports, and concluded that the percentage of false negative predictions increased with the quality of response (FIG. 7A). The 8 cases with moderate/complete response were thus excluded and the prediction accuracy was recalculated, resulting in 73% sensitivity, PPV=35% and NPV=90% and AUC=0.74 (FIG. 7B).

The inventors further examined whether high false predictions are associated with specific conditions, and found high false positive (FP) rates in several gynecological conditions. Specifically, FP rates in women after pregnancy, BRCA-mutation carriers and in women with suspicious pelvic mass were 60%, 35% and 36%, respectively. Of note, 3 out of 4 borderline ovarian tumors were identified as normal, as well as one case of adult granulosa cell tumor (excluded from the analysis) and one case of endometrial carcinoma, which was diagnosed as tumor.

Since the HGOC group is, on average, significantly older than the control group (61.8 vs. 50.5, respectively), and mostly menopausal, whether age and menopausal status are confounders of the proteomic classifier predictions was also tested. Since hormonal status information was not available for all patients, the cohort was divided into age<=50 (pre-menopausal) vs. age>50 (post-menopausal). Multivariate analysis demonstrated a borderline-significant correlation between age or menopausal status and the signature prediction (p-value=0.055 and 0.051, respectively). Reassuringly, the diagnosis of HGOC vs. control strongly correlated with the signature prediction (p-value=0.00019).

### Example 4

Biomarkers Validation by RT-PCR

Some tumor markers (e.g. CA-125) merely reflect an increase in mass of a specific tissue type, and are not exclusively expressed by malignant cells, nor do they possess cancer-promoting biological functions. Such markers are expected to detect tumors only at an advanced stage, and will not be appropriate for early cancer diagnosis. To examine the biological correlate of the proteomic signature, the expression of the signature genes was tested in HGOC tumors vs. normal FTE. The mRNA expression was measured by real time (RT)-PCR on an independent set of unmatched samples: fresh-frozen advanced HGOC tumors (n=10) and unmatched benign FTE cells harvested from normal fimbriae (n=10). The results indicate statistically significant transcriptomic differential expression (DE) of five of the nine genes, in accordance with the proteomic analysis (FIG. 8A-8I). The partial inconsistency between the RT-PCR and the proteomic results may stem from the profound differences in the type of biological materials examined (extracellular microvesicle proteins vs. cellular mRNA), and the methodologies used (MS vs. RT-PCR).

### Example 5

Biomarkers Validation by Immunohistochemistry

MS and RT-PCR methods based on a ‘liquid biopsy’, like UtL, lacks spatial resolution and is unable to disclose the specific cell type expressing each of the classifier's proteins. To explore the localization of the signature proteins in HGOC tumors vs. normal FTE and provide another layer of validity, IHC was performed for selected proteins that were either over-represented (SERPINB5 and S100A14) or under-represented (OVGP1) in UtL of HGOC patients, on a TMA of HGOC tumors vs. 4 control-TMAs representing grossly-normal FT fimbriae removed from women with: HGOC, tubal ectopic pregnancy (EP), leiomyomatous uterus (LM), or BRCA-mutation carriers undergoing RRBSO.

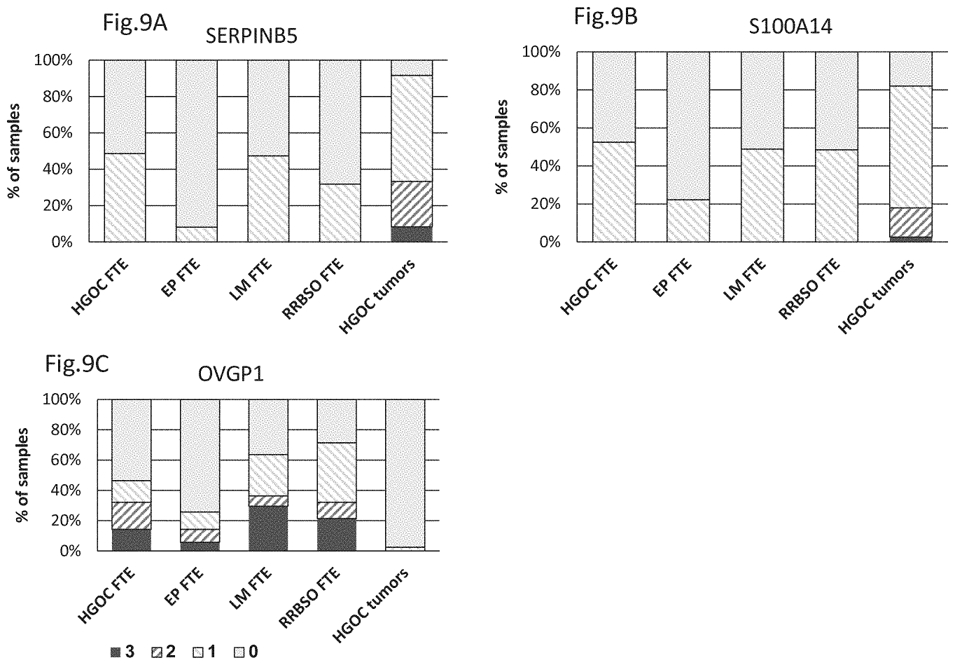

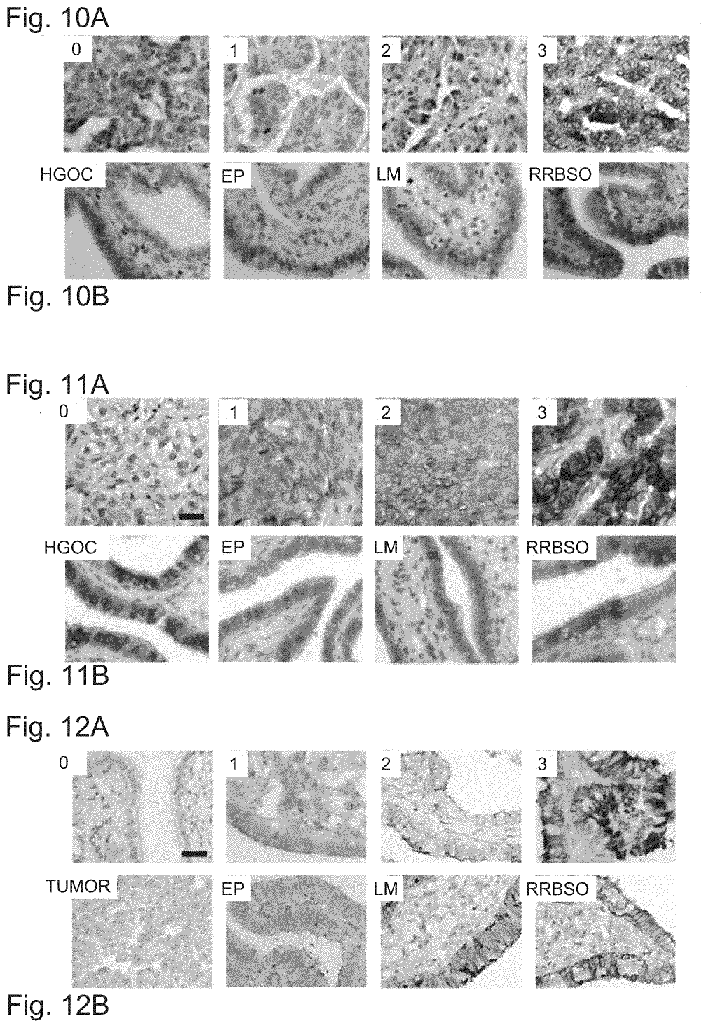

SERPINB5 is an epithelial-cell-specific member of the SERPIN family that lacks serine protease inhibition activity. Not much is known about its cellular functions in cancer, yet it has been implicated as cancer susceptibility gene and a prognostic factor in several cancer types [25]. It has been also attributed a role as an exosomal protein [26]. In accordance with the proteomic analysis, IHC exhibits weak cytoplasmic staining in less than 50% of normal FTE specimens (intensity 0-1), and a stronger expression in a subset of HGOC tumors (FIG. 10A-10B) (p-value=1.65E-09, FIG. 9A).

S100A14 is a member of the S100 family lacking calcium-binding function, known to be involved in the regulation of TP53 protein expression and of cellular motility. In FTE, it localizes exclusively to the cytoplasm of ciliated cells, with very low staining in secretory cells (intensity 0-1) (FIG. 11A-11B). In agreement with the proteomic prediction, its expression was significantly higher in HGOC tumor cells compared to the presumed cell-of-origin: secretory FTE (p-value=8.60E-10, FIG. 9B).

OVGP1 (MUC9) is a mullerian tract specific protein, shown to be elevated in non-serous ovarian tumors [27]. Strongly positive membranous staining is witnessed in most normal FTE, but its expression is decreased in most HGOC tumors (FIG. 12A-12B), probably due to loss of differentiation (p-value=4.59E-17, FIG. 9C).

IHC evidence were further obtained from the Human Protein Atlas database [28] for the expression of three additional proteins. According to this database, CLCA4 (cytoplasmic staining in tumor cells) and CEACAM5 (cytoplasmic/membranous staining in tumor cells) were higher in HGOC, and CLUAP1 showed decreased intensity of cytoplasmic staining in tumor cells. Overall, the IHC results confirm the DE of the signature proteins in HGOC tumors compared to normal FTE, and localize their expression specifically to tumor cells.

### Example 6

Feasibility of Uterine Lavage Procedure for Routine Testing

To further demonstrate clinical feasibility, UtL samples were collected from healthy volunteers who are at high risk for HGOC due to known BRCA mutation (‘high risk cohort’, average age=32.7, n=21). These women underwent the UtL procedure in a clinic setting, without anesthesia. Four women provided 2 UtL samples on consecutive visits, 6 months apart, with 100% concordance. Patient-reported outcomes examined the pain and stress levels, and compliance to undergo the same procedure in subsequent follow-up visits. The average pain score was 1.28 (0 representing no pain (n=12), 5 representing extreme pain (n=2)), and average stress score of 0.8 (0 representing no stress (n=12), 5 representing extreme stress (n=0)). The extra time required to perform the UtL procedure during a routine gynecologic clinic visit was estimated by the participating gynecologists to be5 minutes on average (range 1-10 min, excluding informed consent process).The average UtL sample volume was 5.5 mL, and the average number of proteins identified in these samples was 2600.

Surprisingly, 17 samples (68%) were predicted as ‘tumor’, despite the fact that these donors were asymptomatic, with normal pelvic sonogram and normal CA125 at the time of the examination. The expression of the 9-signature proteins was analyzed in all BRCA mutation carriers samples separately (including patients, controls who provided UtL sample at the time of RRBSO and the high risk cohort), and noticed higher expression of 7 out of 9 signature proteins in the high-risk cohort (FIG. 13). As no pathological correlation is available, these cases are considered FP and warrant further investigation into the underlying molecular aberrations that result in alarming predictions.

1. A diagnostic method for detecting ovarian cancer in a subject, the method comprising:
a. determining the expression level of at least three biomarker proteins in at least one biological sample of said subject, to obtain an expression value for each of said at least three biomarker proteins, wherein said at least three biomarker proteins are selected from Calcium-activated chloride channel regulator 4 (CLCA4), Oviduct-specific glycoprotein (OVGP1), 5100 calcium binding protein A14 (S100A14), Small proline-rich protein 3 (SPRR3), Eosinophil cationic protein (RNASE3), Serpin Family B Member 5 (SERPINB5), Clusterin-associated protein 1 (CLUAP1), Carcinoembryonic antigen-related cell adhesion molecule 5 (CEACAM5) and Ectonucleotide pyrophosphatase/phosphodiesterase family member 3 (ENPP3) or any combination thereof; and
b. determining if the expression value obtained in step (a) for each of said at least three biomarker proteins is positive or negative with respect to a predetermined standard expression value or to an expression value of said biomarker protein/s in at least one control sample;
Wherein at least one of:
(i) a positive expression value of at least one of said SPRR3, SERPINB5, CEACAM5, S100A14 and CLCA4 biomarker protein/s in said sample, indicates that said subject suffers from ovarian cancer; and
(ii) a negative expression value of at least one of said OVGP1, CLUAP1, RNASE3 and ENPP3 biomarker protein/s in said sample, indicates that said subject suffers from ovarian cancer;
optionally, said method further comprises the step of:
c. administering to a subject diagnosed as suffering from ovarian cancer as determined in step (b), a therapeutically effective amount of at least one therapeutic agent.

2. (canceled)

3. The method according to claim 1, wherein determining the level of expression of at least three of said CLCA4, OVGP1, S100A14, SPRR3, RNASE3, SERPINB5, CLUAP1, CEACAM5 and ENPP3 biomarker proteins is performed by the step of contacting at least one detecting molecule or any combination or mixture of plurality of detecting molecules with a biological sample of said subject, or with any protein or nucleic acid product obtained therefrom, wherein each of said detecting molecules is specific for one of said biomarker proteins, wherein said detecting molecule/s is selected from amino acid detecting molecules and nucleic acid detecting molecules.

4. (canceled)

5. The method according to claim 3, wherein said amino acid detecting molecule/s comprise at least one of:
a. at least one labeled or tagged CLCA4, OVGP1, S100A14, SPRR3, RNASE3, SERPINB5, CLUAP1, CEACAM5 and ENPP3 protein/s or any fragment/s, peptide/s or mixture/s thereof;
b. at least one antibody specific for said at least one of said biomarker proteins;
c. at least one protein or peptide aptamer/s specific for said at least one of said biomarker proteins;
d. any combination of (a), (b) and (c).

6-15. (canceled)

16. A diagnostic composition comprising at least one detecting molecule or any combination or mixture of plurality of detecting molecules specific for determining the level of expression of at least three of CLCA4, OVGP1, S100A14, SPRR3, RNASE3, SERPINB5, CLUAP1, CEACAM5 and ENPP3 protein/s or any combination thereof, wherein each of said detecting molecules is specific for one of said biomarker protein/s.

17. (canceled)

18. The composition according to claim 16, wherein said detecting molecules are selected from amino acid detecting molecules and nucleic acid detecting molecules, or any combinations thereof.

19. The composition according to claim 18, wherein said amino acid detecting molecules comprise at least one of:
a. at least one labeled or tagged CLCA4, OVGP1, S100A14, SPRR3, RNASE3, SERPINB5, CLUAP1, CEACAM5 and ENPP3 protein/s or any fragment/s, peptide/s or mixture/s thereof;
b. at least one antibody specific for said at least one of said biomarker protein/s;
c. at least one peptide aptamer/s specific for said at least one biomarker protein/s;
d. any combination of (a), (b) and (c).

20. The composition according to claim 18, wherein said nucleic acid detecting molecule comprise at least one of:
a. at least one nucleic acid aptamer/s specific for said at least one biomarker proteins;
b. at least one oligonucleotide/s, each oligonucleotide specifically hybridizes to a nucleic acid sequence encoding said at least one biomarker protein/s.

21. The composition according to claim 19, wherein:
(a) said detecting molecules are attached to a solid support; or
(b) said detecting molecules are provided in a mixture.

22. The composition according to claim 20, wherein:
(a) said detecting molecules are attached to a solid support; or
(b) said detecting molecules are provided in a mixture.

23. A kit comprising:
a. at least one detecting molecule specific for determining the level of expression of at least three of CLCA4, OVGP1, S100A14, SPRR3, RNASE3, SERPINB5, CLUAP1, CEACAM5 and ENPP3 protein/s or any combination thereof in a biological sample, wherein each of said detecting molecule/s is specific for one of said biomarker proteins; said kit optionally further comprises at least one of:
b. pre-determined calibration curve/s or predetermined standard/s providing standard expression values of said at least one biomarker/s; and
c. at least one control sample.

24. (canceled)

25. The kit according to claim 23, wherein said detecting molecules are selected from amino acid detecting molecule/s, nucleic acid detecting molecule/s, and any combinations thereof.

26. The kit according to claim 25, wherein said amino acid detecting molecules comprise at least one of:
a. at least one labeled or tagged CLCA4, OVGP1, S100A14, SPRR3, RNASE3, SERPINB5, CLUAP1, CEACAM5 and ENPP3 protein/s or any fragment/s, peptide/s or mixture/s thereof;
b. at least one antibody specific for said at least one of said biomarker proteins; and
c. at least one peptide aptamer/s specific for said at least one of said biomarker protein/s;
d. any combination of (a), (b) and (c).

27. The kit according to claim 25, wherein said nucleic acid detecting molecule comprise at least one of:
a. at least one nucleic acid aptamer/s specific for said at least one biomarker proteins;
b. at least one oligonucleotides, each oligonucleotide specifically hybridizes to a nucleic acid sequence encoding said at least one biomarker protein/s.

28. The kit according to claim 24, wherein:
(a) said detecting molecule/s are attached to a solid support; or
(b) said detecting molecule/s is provided in a mixture.

29. (canceled)

30. The kit according to claim 23, further comprising instructions for use, wherein said instructions comprise at least one of:
a. instructions for carrying out the detection and quantification of the expression of said at least one of said CLCA4, OVGP1, S100A14, SPRR3, RNASE3, SERPINB5, CLUAP1, CEACAM5 and ENPP3 biomarker protein/s and optionally, of a control reference protein; and
b. instructions for determining if the expression values of at least one of CLCA4, OVGP1, S100A14, SPRR3, RNASE3, SERPINB5, CLUAP1, CEACAM5 and ENPP3 is positive or negative with respect to a corresponding predetermined standard expression value or with expression value of at least one of said biomarker protein/s in said at least one control sample.

31. The kit according to claim 23, further comprising at least one of:
(a) at least one reagent for conducting a mass spectrometry assay; and
(b) at least one reagent for conducting an immunological assay.

32. (canceled)

33. The kit according to claim 23, further comprising at least one device or means for obtaining a body fluid sample and for isolating microvesicles from said body fluid sample.

34. The kit according to claim 23, wherein said kit is adapted for use in a method for detecting ovarian cancer in a subject.

35-36. (canceled)

37. The kit according to claim 23, wherein said sample is a body fluid sample, optionally, said sample is microvesicles prepared from said body fluid.

38. (canceled)

39. The kit according to claim 37, wherein said body fluid is at least one of uterine lavage fluid (UtLF) and plasma, optionally, wherein said sample comprises microvesicles isolated from UtLF.

40. (canceled)


SEQUENCE LISTING (74 sequences)

SEQ ID NO: 1 - protein, 678 aa, Homo sapiens
  MISC_FEATURE: OVGP1 UNITPROT ID Q12889
MWKLLLWVGLVLVLKHHDGAAHKLVCYFTNWAHSRPGPASILPHDLDPFLCTHLIFAFASMNNNQIVAKDLQDEKILYPEFNKLKERNRELKTLLSIGGWNFGTSRFTTMLSTFANREKFIASVISLLRTHDFDGLDLFFLYPGLRGSPMHDRWTFLFLIEELLFAFRKEALLTMRPRLLLSAAVSGVPHIVQTSYDVRFLGRLLDFINVLSYDLHGSWERFTGHNSPLFSLPEDPKSSAYAMNYWRKLGAPSEKLIMGIPTYGRTFRLLKASKNGLQARAIGPASPGKYTKQEGFLAYFEICSFVWGAKKHWIDYQYVPYANKGKEWVGYDNAISFSYKAWFIRREHFGGAMVWTLDMDDVRGTFCGTGPFPLVYVLNDILVRAEFSSTSLPQFWLSSAVNSSSTDPERLAVTTAWTTDSKILPPGGEAGVTEIHGKCENMTITPRGTTVTPTKETVSLGKHTVALGEKTEITGAMTMTSVGHQSMTPGEKALTPVGHQSVTTGQKTLTSVGYQSVTPGEKTLTPVGHQSVTPVSHQSVSPGGTTMTPVHFQTETLRQNTVAPRRKAVAREKVTVPSRNISVTPEGQTMPLRGENLTSEVGTHPRMGNLGLQMEAENRMMLSSSPVIQLPEQTPLAFDNRFVPIYGNHSSVNSVTPQTSPLSLKKEIPENSAVDEEA

SEQ ID NO: 2 - DNA, 2037 bp, Homo sapiens
  misc_feature: OVGP1 Accession number NP_002548.3
atgtggaagctgttgctgtgggttgggctggttcttgtgctgaaacaccacgatggtgctgcccataaactcgtgtgttatttcaccaactgggcacacagtcggccaggccctgcctcgatcttgccccatgacctggacccctttctctgcacccacctgatatttgcctttgcctcaatgaacaacaatcagattgttgctaaggatctccaggatgagaaaattctctacccagagttcaacaaactaaaggagaggaacagagagctgaaaacactactgtccatcggcgggtggaactttggcacctcaagattcaccactatgttgtccacatttgccaaccgtgaaaagtttattgcttcagttatatcccttctgaggacacatgactttgatggtcttgaccttttcttcttatatcctggactaagaggcagccccatgcatgaccggtggacttttctcttcttaattgaagagctcctgtttgccttccggaaggaggcactgctcaccatgcgcccgaggctgctgctgtctgctgctgtttctggggtcccacacatcgtccaaacatcctatgatgtgcgctttctaggaagactcctggatttcatcaatgtcttgtcttatgacttacatggaagttgggaaaggttcacaggacataatagccccctcttctctctgcctgaagaccccaaatcttcggcatatgctatgaattattggagaaagcttggggcaccctcagagaagctcatcatggggatccccacctatggacgtacctttcgcctcctcaaagcctctaagaatgggttgcaggccagagcgatcggaccagcatctccagggaagtacaccaagcaagaaggcttcttggcttattttgagatttgttcctttgtctggggagcgaagaagcactggattgattaccagtatgtcccgtatgccaacaaggggaaagagtgggttggctatgacaatgccatcagcttcagttacaaggcatggtttataaggcgagagcattttgggggggccatggtgtggacattggacatggatgacgtcaggggcacgttctgtggcactggccctttcccccttgtctacgtattgaatgatatcctggtgcgggctgagttcagttcaacttctttaccacaattttggctgtcatctgctgtgaattcttcaagcactgaccctgaaaggctggctgtgaccacggcatggaccactgatagtaagattttgcccccaggaggagaggctggggtcactgagatccacggaaagtgtgaaaatatgactataacccctagaggtacaactgtgacccctacaaaggaaactgtatcccttggaaagcacactgtagctctaggagagaagactgagatcactggggcaatgaccatgacttctgtgggtcatcagtccatgacccctggagagaaggccctgacccctgtgggtcatcaatctgtgaccactggacagaagaccctgacctctgtgggttatcagtctgtgacccctggggaaaagaccctgacccctgtgggtcatcagtctgtgacccctgtgagtcatcagtctgtgagccctggaggaacgactatgacccctgtccattttcagactgagacccttagacagaatacagtggcccctagaaggaaggctgtggcccgtgaaaaggtgactgtcccctccagaaacatatcagtcacccctgaagggcagactatgcctttaagaggggagaatttgacttctgaggtgggcactcaccccaggatgggtaacttgggtcttcagatggaagctgaaaacaggatgatgctgtcctccagccccgtcatccagctcccggaacaaactcctctagcttttgacaaccgctttgttcccatctatggaaaccattcctctgtcaactcagtaacccctcaaacaagtcctctttctctaaaaaaagaaatcccagaaaactctgctgtggatgaagaagcctaa

SEQ ID NO: 3 - protein, 169 aa, Homo sapiens
  MISC_FEATURE: SPRR3  UNITPROT ID Q9UBC9
MSSYQQKQTFTPPPQLQQQQVKQPSQPPPQEIFVPTTKEPCHSKVPQPGNTKIPEPGCTKVPEPGCTKVPEPGCTKVPEPGCTKVPEPGCTKVPEPGCTKVPEPGYTKVPEPGSIKVPDQGFIKFPEPGAIKVPEQGYTKVPVPGYTKLPEPCPSTVTPGPAQQKTKQK

SEQ ID NO: 4 - DNA, 574 bp, Homo sapiens
  misc_feature: SPRR3  Accession number AK311823.1
accagatcccagaggctgaacacctcgaccttctctgcacagcaggtccagcatcctttgaagcatgagttcttaccagcagaagcagacctttaccccaccacctcagcttcaacagcagcaggtgaaacaacccagccagcctccacctcaggaaatatttgttcccacaaccaaggagccatgccactcaaaggttccacaacctggaaacacaaagattccagagccaggctgtaccaaggtccctgagccaggctgtaccaaggtccctgagccaggctgtaccaaggtccctgagccaggttgtaccaaggtccctgagccaggctgtaccaaggtccctgagccaggttgtaccaaggtccctgagccaggctacaccaaggtccctgaaccaggcagcatcaaggtccctgaccaaggcttcatcaagtttcctgagccaggtgccatcaaagttcctgagcaaggatacaccaaagttcctgtgccaggctacacaaagctaccagagccatgtccttcaacggtcactccaggcccagctcagcagaagaccaagcagaagtaa

SEQ ID NO: 5 - protein, 919 aa, Homo sapiens
  MISC_FEATURE: CLCA4  UNITPROT ID Q14CN2
MGLFRGFVFLLVLCLLHQSNTSFIKLNNNGFEDIVIVIDPSVPEDEKIIEQIEDMVTTASTYLFEATEKRFFFKNVSILIPENWKENPQYKRPKHENHKHADVIVAPPTLPGRDEPYTKQFTECGEKGEYIHFTPDLLLGKKQNEYGPPGKLFVHEWAHLRWGVFDEYNEDQPFYRAKSKKIEATRCSAGISGRNRVYKCQGGSCLSRACRIDSTTKLYGKDCQFFPDKVQTEKASIMFMQSIDSVVEFCNEKTHNQEAPSLQNIKCNFRSTWEVISNSEDFKNTIPMVTPPPPPVFSLLKISQRIVCLVLDKSGSMGGKDRLNRMNQAAKHFLLQTVENGSWVGMVHFDSTATIVNKLIQIKSSDERNTLMAGLPTYPLGGTSICSGIKYAFQVIGELHSQLDGSEVLLLTDGEDNTASSCIDEVKQSGAIVHFIALGRAADEAVIEMSKITGGSHFYVSDEAQNNGLIDAFGALTSGNTDLSQKSLQLESKGLTLNSNAWMNDTVIIDSTVGKDTFFLITWNSLPPSISLWDPSGTIMENFTVDATSKMAYLSIPGTAKVGTWAYNLQAKANPETLTITVTSRAANSSVPPITVNAKMNKDVNSFPSPMIVYAEILQGYVPVLGANVTAFIESQNGHTEVLELLDNGAGADSFKNDGVYSRYFTAYTENGRYSLKVRAHGGANTARLKLRPPLNRAAYIPGWVVNGEIEANPPRPEIDEDTQTTLEDFSRTASGGAFVVSQVPSLPLPDQYPPSQITDLDATVHEDKIILTWTAPGDNFDVGKVQRYIIRISASILDLRDSFDDALQVNTTDLSPKEANSKESFAFKPENISEENATHIFIAIKSIDKSNLTSKVSNIAQVTLFIPQANPDDIDPTPTPTPTPTPDKSHNSGVNISTLVLSVIGSVVIVNFILSTTI

SEQ ID NO: 6 - DNA, 2760 bp, Homo sapiens
  misc_feature: CLCA4 Accession number NM_012128.3
atggggttattcagaggttttgttttcctcttagttctgtgcctgctgcaccagtcaaatacttccttcattaagctgaataataatggctttgaagatattgtcattgttatagatcctagtgtgccagaagatgaaaaaataattgaacaaatagaggatatggtgactacagcttctacgtacctgtttgaagccacagaaaaaagattttttttcaaaaatgtatctatattaattcctgagaattggaaggaaaatcctcagtacaaaaggccaaaacatgaaaaccataaacatgctgatgttatagttgcaccacctacactcccaggtagagatgaaccatacaccaagcagttcacagaatgtggagagaaaggcgaatacattcacttcacccctgaccttctacttggaaaaaaacaaaatgaatatggaccaccaggcaaactgtttgtccatgagtgggctcacctccggtggggagtgtttgatgagtacaatgaagatcagcctttctaccgtgctaagtcaaaaaaaatcgaagcaacaaggtgttccgcaggtatctctggtagaaatagagtttataagtgtcaaggaggcagctgtcttagtagagcatgcagaattgattctacaacaaaactgtatggaaaagattgtcaattctttcctgataaagtacaaacagaaaaagcatccataatgtttatgcaaagtattgattctgttgttgaattttgtaacgaaaaaacccataatcaagaagctccaagcctacaaaacataaagtgcaattttagaagtacatgggaggtgattagcaattctgaggattttaaaaacaccatacccatggtgacaccacctcctccacctgtcttctcattgctgaagatcagtcaaagaattgtgtgcttagttcttgataagtctggaagcatggggggtaaggaccgcctaaatcgaatgaatcaagcagcaaaacatttcctgctgcagactgttgaaaatggatcctgggtggggatggttcactttgatagtactgccactattgtaaataagctaatccaaataaaaagcagtgatgaaagaaacacactcatggcaggattacctacatatcctctgggaggaacttccatctgctctggaattaaatatgcatttcaggtgattggagagctacattcccaactcgatggatccgaagtactgctgctgactgatggggaggataacactgcaagttcttgtattgatgaagtgaaacaaagtggggccattgttcattttattgctttgggaagagctgctgatgaagcagtaatagagatgagcaagataacaggaggaagtcatttttatgtttcagatgaagctcagaacaatggcctcattgatgcttttggggctcttacatcaggaaatactgatctctcccagaagtcccttcagctcgaaagtaagggattaacactgaatagtaatgcctggatgaacgacactgtcataattgatagtacagtgggaaaggacacgttctttctcatcacatggaacagtctgcctcccagtatttctctctgggatcccagtggaacaataatggaaaatttcacagtggatgcaacttccaaaatggcctatctcagtattccaggaactgcaaaggtgggcacttgggcatacaatcttcaagccaaagcgaacccagaaacattaactattacagtaacttctcgagcagcaaattcttctgtgcctccaatcacagtgaatgctaaaatgaataaggacgtaaacagtttccccagcccaatgattgtttacgcagaaattctacaaggatatgtacctgttcttggagccaatgtgactgctttcattgaatcacagaatggacatacagaagttttggaacttttggataatggtgcaggcgctgattctttcaagaatgatggagtctactccaggtattttacagcatatacagaaaatggcagatatagcttaaaagttcgggctcatggaggagcaaacactgccaggctaaaattacggcctccactgaatagagccgcgtacataccaggctgggtagtgaacggggaaattgaagcaaacccgccaagacctgaaattgatgaggatactcagaccaccttggaggatttcagccgaacagcatccggaggtgcatttgtggtatcacaagtcccaagccttcccttgcctgaccaatacccaccaagtcaaatcacagaccttgatgccacagttcatgaggataagattattcttacatggacagcaccaggagataattttgatgttggaaaagttcaacgttatatcataagaataagtgcaagtattcttgatctaagagacagttttgatgatgctcttcaagtaaatactactgatctgtcaccaaaggaggccaactccaaggaaagctttgcatttaaaccagaaaatatctcagaagaaaatgcaacccacatatttattgccattaaaagtatagataaaagcaatttgacatcaaaagtatccaacattgcacaagtaactttgtttatccctcaagcaaatcctgatgacattgatcctacacctactcctactcctactcctactcctgataaaagtcataattctggagttaatatttctacgctggtattgtctgtgattgggtctgttgtaattgttaactttattttaagtaccaccatttga

SEQ ID NO: 7 - protein, 104 aa, Homo sapiens
  MISC_FEATURE: S100A14 Accession number NM_020672
MGQCRSANAEDAQEFSDVERAIETLIKNFHQYSVEGGKETLTPSELRDLVTQQLPHLMPSNCGLEEKIANLGSCNDSKLEFRSFWELIGEAAKSVKLERPVRGH

SEQ ID NO: 8 - DNA, 1075 bp, Homo sapiens
  misc_feature: cDNA  S100A14
gctggctcctcctgtcttgtctcagcggctgccaacagatcatgagccatcagctcctctggggccagctataggacaacagaactctcaccaaaggaccagacacagtgggcaccatgggacagtgtcggtcagccaacgcagaggatgctcaggaattcagtgatgtggagagggccattgagaccctcatcaagaactttcaccagtactccgtggagggtgggaaggagacgctgaccccttctgagctacgggacctggtcacccagcagctgccccatctcatgccgagcaactgtggcctggaagagaaaattgccaacctgggcagctgcaatgactctaaactggagttcaggagtttctgggagctgattggagaagcggccaagagtgtgaagctggagaggcctgtccgggggcactgagaactccctctggaattcttggggggtgttggggagagactgtgggcctggagataaaacttgtctcctctaccaccaccctgtaccctagcctgcacctgtcctcatctctgcaaagttcagcttccttccccaggtctctgtgcactctgtcttggatgctctggggagctcatgggtggaggagtctccaccagagggaggctcaggggactggttgggccagggatgaatatttgagggataaaaattgtgtaagagccaaagaattggtagtagggggagaacagagaggagctgggctatgggaaatgatttgaataatggagctgggaatatggctggatatctggtactaaaaaagggtctttaagaacctacttcctaatctcttccccaatccaaaccatagctgtctgtccagtgctctcttcctgcctccagctctgccccaggctcctcctagactctgtccctgggctagggcaggggaggagggagagcagggttgggggagaggctgaggagagtgtgacatgtggggagaggaccagctgggtgcttgggcattgacagaatgatggttgttttgtatcatttgattaataaaaaaaaatgaaaaaagtgaaaaaaaaaaaaaaaaa

SEQ ID NO: 9 - protein, 413 aa, Homo sapiens
  MISC_FEATURE: CLUAP1 UNITPROT ID Q96AJ1
MSFRDLRNFTEMMRALGYPRHISMENFRTPNFGLVSEVLLWLVKRYEPQTDIPPDVDTEQDRVFFIKAIAQFMATKAHIKLNTKKLYQADGYAVKELLKITSVLYNAMKTKGMEGSEIVEEDVNKFKFDLGSKIADLKAARQLASEITSKGASLYDLLGMEVELREMRTEAIARPLEINETEKVMRIAIKEILTQVQKTKDLLNNVASDEANLEAKIEKRKLELERNRKRLETLQSVRPCFMDEYEKTEEELQKQYDTYLEKFQNLTYLEQQLEDHHRMEQERFEEAKNTLCLIQNKLKEEEKRLLKSGSNDDSDIDIQEDDESDSELEERRLPKPQTAMEMLMQGRPGKRIVGTMQGGDSDDNEDSEESEIDMEDDDDEDDDLEDESISLSPTKPNRRVRKSEPLDESDNDF

SEQ ID NO: 10 - DNA, 1242 bp, Homo sapiens
  misc_feature: CLUAP1  Accession number NM_015041.2
atgtctttccgcgacctccgcaatttcacagagatgatgagagccctgggataccctcgacatatttctatggaaaatttccgtacacccaattttggacttgtatctgaagtgcttctctggcttgtgaaaagatatgagccccagactgacatcccgcctgacgtggatactgaacaggaccgagttttcttcattaaggcaattgcccagttcatggccaccaaggcacatataaaactcaacactaagaagctttatcaagcagatgggtatgcggtaaaagagctgctgaagatcacatctgtcctttataatgctatgaagaccaaggggatggagggctctgaaatagtagaggaagatgtcaacaagttcaagtttgatcttggctcaaagattgcagatttgaaggcagccaggcagcttgcgtctgaaatcacctccaaaggagcatctctgtatgacttgctcggcatggaagtagagttgagggaaatgagaacagaagccattgccagacctctggaaataaacgagactgaaaaagtgatgagaattgcaataaaagagattttgacacaggttcagaagactaaagacctgctcaataatgtggcctctgatgaagctaatttagaagccaaaatcgaaaagagaaaattagaactggaaagaaatcggaagcgactagagactctgcagagtgtcaggccatgttttatggatgagtatgagaagactgaggaagaattacaaaagcagtatgacacttatctggagaaatttcaaaatctgacttatctggaacaacagcttgaagaccatcataggatggagcaagaaaggtttgaggaagctaaaaacactctctgcctgatacagaacaagctcaaggaggaagagaagcgcctgctcaagagtggaagtaacgatgactcggacatagacatccaggaggacgatgaatccgacagtgagttggaagaaaggcggctgcccaagccacagacagccatggagatgctcatgcaaggaagacctggcaaacgcattgtgggcacgatgcaaggtggagactccgatgacaatgaggactcggaggagagtgaaattgacatggaagatgatgatgacgaggatgacgatttggaagacgagagcatttctctctcaccaaccaagcccaatcgaagggtccggaaatctgaacccctggatgagagtgacaatgacttctga

SEQ ID NO: 11 - protein, 375 aa, Homo sapiens
  MISC_FEATURE: SERPINB5  ACCESSION   NM_002639
MDALQLANSAFAVDLFKQLCEKEPLGNVLFSPICLSTSLSLAQVGAKGDTANEIGQVLHFENVKDVPFGFQTVTSDVNKLSSFYSLKLIKRLYVDKSLNLSTEFISSTKRPYAKELETVDFKDKLEETKGQINNSIKDLTDGHFENILADNSVNDQTKILVVNAAYFVGKWMKKFSESETKECPFRVNKTDTKPVQMMNMEATFCMGNIDSINCKIIELPFQNKHLSMFILLPKDVEDESTGLEKIEKQLNSESLSQWTNPSTMANAKVKLSIPKFKVEKMIDPKACLENLGLKHIFSEDTSDFSGMSETKGVALSNVIHKVCLEITEDGGDSIEVPGARILQHKDELNADHPFIYIIRHNKTRNIIFFGKFCSP

SEQ ID NO: 12 - DNA, 2633 bp, Homo sapiens
  misc_feature: cDNA SERPINB5
agtgggcgtggcggtgctgcccaggtgagccaccgctgcttctgcccagacacggtcgcctccacatccaggtctttgtgctcctcgcttgcctgttccttttccacgcattttccaggataactgtgactccaggcccgcaatggatgccctgcaactagcaaattcggcttttgccgttgatctgttcaaacaactatgtgaaaaggagccactgggcaatgtcctcttctctccaatctgtctctccacctctctgtcacttgctcaagtgggtgctaaaggtgacactgcaaatgaaattggacaggttcttcattttgaaaatgtcaaagatgtaccctttggatttcaaacagtaacatcggatgtaaacaaacttagttccttttactcactgaaactaatcaagcggctctacgtagacaaatctctgaatctttctacagagttcatcagctctacgaagagaccgtatgcaaaggaattggaaactgttgacttcaaagataaattggaagaaacgaaaggtcagatcaacaactcaattaaggatctcacagatggccactttgagaacattttagctgacaacagtgtgaacgaccagaccaaaatccttgtggttaatgctgcctactttgttggcaagtggatgaagaaattttctgaatcagaaacaaaagaatgtcctttcagagtcaacaagacagacaccaaaccagtgcagatgatgaacatggaggccacgttctgtatgggaaacattgacagtatcaattgtaagatcatagagcttccttttcaaaataagcatctcagcatgttcatcctactacccaaggatgtggaggatgagtccacaggcttggagaagattgaaaaacaactcaactcagagtcactgtcacagtggactaatcccagcaccatggccaatgccaaggtcaaactctccattccaaaatttaaggtggaaaagatgattgatcccaaggcttgtctggaaaatctagggctgaaacatatcttcagtgaagacacatctgatttctctggaatgtcagagaccaagggagtggccctatcaaatgttatccacaaagtgtgcttagaaataactgaagatggtggggattccatagaggtgccaggagcacggatcctgcagcacaaggatgaattgaatgctgaccatccctttatttacatcatcaggcacaacaaaactcgaaacatcattttctttggcaaattctgttctccttaagtggcatagcccatgttaagtcctccctgacttttctgtggatgccgatttctgtaaactctgcatccagagattcattttctagatacaataaattgctaatgttgctggatcaggaagccgccagtacttgtcatatgtagccttcacacagatagaccttttttttttttccaattctatcttttgtttccttttttcccataagacaatgacatacgcttttaatgaaaaggaatcacgttagaggaaaaatatttattcattatttgtcaaattgtccggggtagttggcagaaatacagtcttccacaaagaaaattcctataaggaagatttggaagctcttcttcccagcactatgctttccttctttgggatagagaatgttccagacattctcgcttccctgaaagactgaagaaagtgtagtgcatgggacccacgaaactgccctggctccagtgaaacttgggcacatgctcaggctactataggtccagaagtccttatgttaagccctggcaggcaggtgtttattaaaattctgaattttggggattttcaaaagataatattttacatacactgtatgttatagaacttcatggatcagatctggggcagcaccctataaatcaacaccttaatatgctgcaacaaaatgtagaatattcagacaaaatggatacataaagactaagtagcccataaggggtcaaaatttgctgccaaatgcgtatgccaccaacttacaaaaacacttcgttcgcagagcttttcagattgtggaatgttggataaggaattatagacctctagtagctgaaatgcaagaccccaagaggaagttcagatcttaatataaattcactttcatttttgatagctgtcccatctggtcatttggttggcactagactggtggcaggggcttctagctgacttgcacagggattctcacaatagccgatatcagaatttgtgttgaaggaacttgtctcttcatctaatatgatagcgggaaaaggagaggaaactactgcctttagaaaatataagtaaagtgattaaagtgctcacgttaccttgacacatagtttttcagtctatgggtttagttactttagatggcaagcatgtaacttatattaatagtaatttgtaaagttggttggataagctatccgtgttgcaggttcatggattacttctctataaaaaatatgtatttaccaaaaattttgtgacattccttctcccatctcttccttgacctgcattgtaaataggttcttcttgttctgagattcaatattgaatttttcctatgctattgacaataaaatattattgaactaca

SEQ ID NO: 13 - protein, 160 aa, Homo sapiens
  MISC_FEATURE: RNASE3  UNITPROT ID P12724
MVPKLFTSQICLLLLLGLMGVEGSLHARPPQFTRAQWFAIQHISLNPPRCTIAMRAINNYRWRCKNQNTFLRTTFANVVNVCGNQSIRCPHNRTLNNCHRSRFRVPLLHCDLINPGAQNISNCTYADRPGRRFYVVACDNRDPRDSPRYPVVPVHLDTTI

SEQ ID NO: 14 - DNA, 483 bp, Homo sapiens
  misc_feature: RNASE3  Accession number NP_002926.2
atggttccaaaactgttcacttcccaaatttgtctgcttcttctgttggggcttatgggtgtggagggctcactccatgccagacccccacagtttacgagggctcagtggtttgccatccagcacatcagtctgaacccccctcgatgcaccattgcaatgcgggcaattaacaattatcgatggcgttgcaaaaaccaaaatacttttcttcgtacaacttttgctaatgtagttaatgtttgtggtaaccaaagtatacgctgccctcataacagaactctcaacaattgtcatcggagtagattccgggtgcctttactccactgtgacctcataaatccaggtgcacagaatatttcaaactgcacgtatgcagacagaccaggaaggaggttctatgtagttgcatgtgacaacagagatccacgggattctccacggtatcctgtggttccagttcacctggataccaccatctaa

SEQ ID NO: 15 - protein, 702 aa, Homo sapiens
  MISC_FEATURE: CEACAM5  UNITPROT ID P06731, Accession number NP_001278413.1
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 16 - DNA, 2109 bp, Homo sapiens
  misc_feature: CEACAM5  UNITPROT ID P06731, Accession number NP_001278413.1
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 17 - protein, 875 aa, Homo sapiens [sequence omitted]

SEQ ID NO: 18 - DNA, 2628 bp, Homo sapiens
  misc_feature: ENPP3 UNITPROT ID O14638, Accession number NP_005012.2
atggaatctacgttgactttagcaacggaacaacctgttaagaagaacactcttaagaaatataaaatagcttgcattgttcttcttgctttgctggtgatcatgtcacttggattaggcctggggcttggactcaggaaactggaaaagcaaggcagctgcaggaagaagtgctttgatgcatcatttagaggactggagaactgccggtgtgatgtggcatgtaaagaccgaggtgattgctgctgggattttgaagacacctgtgtggaatcaactcgaatatggatgtgcaataaatttcgttgtggagagaccagattagaggccagcctttgctcttgttcagatgactgtttgcagaggaaagattgctgtgctgactataagagtgtttgccaaggagaaacctcatggctggaagaaaactgtgacacagcccagcagtctcagtgcccagaagggtttgacctgccaccagttatcttgttttctatggatggatttagagctgaatatttatacacatgggatactttaatgccaaatatcaataaactgaaaacatgtggaattcattcaaaatacatgagagctatgtatcctaccaaaaccttcccaaatcattacaccattgtcacgggcttgtatccagagtcacatggcatcattgacaataatatgtatgatgtaaatctcaacaagaatttttcactttcttcaaaggaacaaaataatccagcctggtggcatgggcaaccaatgtggctgacagcaatgtatcaaggtttaaaagccgctacctacttttggcccggatcagaagtggctataaatggctcctttccttccatatacatgccttacaacggaagtgtcccatttgaagagaggatttctacactgttaaaatggctggacctgcccaaagctgaaagacccaggttttataccatgtattttgaagaacctgattcctctggacatgcaggtggaccagtcagtgccagagtaattaaagccttacaggtagtagatcatgcttttgggatgttgatggaaggcctgaagcagcggaatttgcacaactgtgtcaatatcatccttctggctgaccatggaatggaccagacttattgtaacaagatggaatacatgactgattattttcccagaataaacttcttctacatgtacgaagggcctgccccccgcatccgagctcataatatacctcatgacttttttagttttaattctgaggaaattgttagaaacctcagttgccgaaaacctgatcagcatttcaagccctatttgactcctgatttgccaaagcgactgcactatgccaagaacgtcagaatcgacaaagttcatctctttgtggatcaacagtggctggctgttaggagtaaatcaaatacaaattgtggaggaggcaaccatggttataacaatgagtttaggagcatggaggctatctttctggcacatggacccagttttaaagagaagactgaagttgaaccatttgaaaatattgaagtctataacctaatgtgtgatcttctacgcattcaaccagcaccaaacaatggaacccatggtagtttaaaccatcttctgaaggtgcctttttatgagccatcccatgcagaggaggtgtcaaagttttctgtttgtggctttgctaatccattgcccacagagtctcttgactgtttctgccctcacctacaaaatagtactcagctggaacaagtgaatcagatgctaaatctcacccaagaagaaataacagcaacagtgaaagtaaatttgccatttgggaggcctagggtactgcagaagaacgtggaccactgtctcctttaccacagggaatatgtcagtggatttggaaaagctatgaggatgcccatgtggagttcatacacagtcccccagttgggagacacatcgcctctgcctcccactgtcccagactgtctgcgggctgatgtcagggttcctccttctgagagccaaaaatgttccttctatttagcagacaagaatatcacccacggcttcctctatcctcctgccagcaatagaacatcagatagccaatatgatgctttaattactagcaatttggtacctatgtatgaagaattcagaaaaatgtgggactacttccacagtgttcttcttataaaacatgccacagaaagaaatggagtaaatgtggttagtggaccaatatttgattataattatgatggccattttgatgctccagatgaaattaccaaacatttagccaacactgatgttcccatcccaacacactactttgtggtgctgaccagttgtaaaaacaagagccacacaccggaaaactgccctgggtggctggatgtcctaccctttatcatccctcaccgacctaccaacgtggagagctgtcctgaaggtaaaccagaagctctttgggttgaagaaagatttacagctcacattgcccgggtccgtgatgtagaacttctcactgggcttgacttctatcaggataaagtgcagcctgtctctgaaattttgcaactaaagacatatttaccaacatttgaaaccactatttaa

SEQ ID NO: 19 - protein, 344 aa, Homo sapiens
  MISC_FEATURE: CEACAM6  ACCESSION  NM_002483
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 20 - DNA, 2601 bp, Homo sapiens
  misc_feature: cDNA CEACAM6
gaggctcagcacagaaggaggaaggacagcagggccaacagtcacagcagccctgaccagagcattcctggagctcaagctcctctacaaagaggtggacagagaagacagcagagaccatgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggaagtgctcctgtcctctcagctgtggccaccgtcggcatcacgattggagtgctggccagggtggctctgatatagcagccctggtgtattttcgatatttcaggaagactggcagattggaccagaccctgaattcttctagctcctccaatcccattttatcccatggaaccactaaaaacaaggtctgctctgctcctgaagccctatatgctggagatggacaactcaatgaaaatttaaagggaaaaccctcaggcctgaggtgtgtgccactcagagacttcacctaactagagacagtcaaactgcaaaccatggtgagaaattgacgacttcacactatggacagcttttcccaagatgtcaaaacaagactcctcatcatgataaggctcttacccccttttaatttgtccttgcttatgcctgcctctttcgcttggcaggatgatgctgtcattagtatttcacaagaagtagcttcagagggtaacttaacagagtgtcagatctatcttgtcaatcccaacgttttacataaaataagagatcctttagtgcacccagtgactgacattagcagcatctttaacacagccgtgtgttcaaatgtacagtggtccttttcagagttggacttctagactcacctgttctcactccctgttttaattcaacccagccatgcaatgccaaataatagaattgctccctaccagctgaacagggaggagtctgtgcagtttctgacacttgttgttgaacatggctaaatacaatgggtatcgctgagactaagttgtagaaattaacaaatgtgctgcttggttaaaatggctacactcatctgactcattctttattctattttagttggtttgtatcttgcctaaggtgcgtagtccaactcttggtattaccctcctaatagtcatactagtagtcatactccctggtgtagtgtattctctaaaagctttaaatgtctgcatgcagccagccatcaaatagtgaatggtctctctttggctggaattacaaaactcagagaaatgtgtcatcaggagaacatcataacccatgaaggataaaagccccaaatggtggtaactgataatagcactaatgctttaagatttggtcacactctcacctaggtgagcgcattgagccagtggtgctaaatgctacatactccaactgaaatgttaaggaagaagatagatccaattaaaaaaaattaaaaccaatttaaaaaaaaaaagaacacaggagattccagtctacttgagttagcataatacagaagtcccctctactttaacttttacaaaaaagtaacctgaactaatctgatgttaaccaatgtatttatttctgtggttctgtttccttgttccaatttgacaaaacccactgttcttgtattgtattgcccagggggagctatcactgtacttgtagagtggtgctgctttaattcataaatcacaaataaaagccaattagctctataact

SEQ ID NO: 21 - protein, 136 aa, Homo sapiens
  MISC_FEATURE: LGALS7 ACCESSION   NM_002307
MSNVPHKSSLPEGIRPGTVLRIRGLVPPNASRFHVNLLCGEEQGSDAALHFNPRLDTSEVVFNSKEQGSWGREERGPGVPFQRGQPFEVLIIASDDGFKAVVGDAQYHHFRHRLPLARVRLVEVGGDVQLDSVRIF

SEQ ID NO: 22 - DNA, 515 bp, Homo sapiens
  misc_feature: cDNA LGALS7
acggctgcccaacccggtcccagccatgtccaacgtcccccacaagtcctcactgcccgagggcatccgccctggcacggtgctgagaattcgcggcttggttcctcccaatgccagcaggttccatgtaaacctgctgtgcggggaggagcagggctccgatgccgcgctgcatttcaacccccggctggacacgtcggaggtggtcttcaacagcaaggagcaaggctcctggggccgcgaggagcgcgggccgggcgttcctttccagcgcgggcagcccttcgaggtgctcatcatcgcgtcagacgacggcttcaaggccgtggttggggacgcccagtaccaccacttccgccaccgcctgccgctggcgcgcgtgcgcctggtggaggtgggcggggacgtgcagctggactccgtgaggatcttctgagcagaagcccaggcgggcccggggccttggctggcaaataaagcgttagcccgcagcgaaaaaaaaaaaaaaaaaaaaa

SEQ ID NO: 23 - protein, 349 aa, Homo sapiens
  MISC_FEATURE: BCAT1 Accession number NM_001178091
MKDCSNGCSAECTGEGGSKEVVGTFKAKDLIVTPATILKEKPDPNNLVFGTVFTDHMLTVEWSSEFGWEKPHIKPLQNLSLHPGSSALHYAVEVFDKEELLECIQQLVKLDQEWVPYSTSASLYIRPTFIGTEPSLGVKKPTKALLFVLLSPVGPYFSSGTFNPVSLWANPKYVRAWKGGTGDCKMGGNYGSSLFAQCEAVDNGCQQVLWLYGEDHQITEVGTMNLFLYWINEDGEEELATPPLDGIILPGVTRRCILDLAHQWGEFKVSERYLTMDDLTTALEGNRVREMFGSGTACVVCPVSDILYKGETIHIPTMENGPKLASRILSKLTDIQYGREESDWTIVLS

SEQ ID NO: 24 - DNA, 9571 bp, Homo sapiens
  misc_feature: cDNA BCAT1
agtagggaggtgggcaggagccagtgatgacggaatggcaatcacatttgacctctgatctgtttatttcctcctccttgacgtctccatataaatgttacacgggcatccccacactcggatacgcacccacagtggctgattcgggggtaaccgtgtcatttgcttgcaacactggcacctctgccctgcaccccgggagtgagcagtgagtgaggctcgggtctgggcgctggctccgaatcttcgggctgggagagactccaccatctgggggcggcctgggggagcagccttagtgtcttcctgctgatgcaatccgctaggtcgcgagtctccgccgcgagagggccggtctgcaatccagcccgccacgtgtactcgccgccgcctcgggcactgccccaggtcttgctgcagccgggaccgcgctctgcagccgcagacccggtccacacggccaggggctacgacccttgggatctgccctccgctcagctcgagcttccctcgtggccgacggaacaatgaaggattgcagtaacggatgctccgcagagtgtaccggagaaggaggatcaaaagaggtggtggggacttttaaggctaaagacctaatagtcacaccagctaccattttaaaggaaaaaccagaccccaataatctggtttttggaactgtgttcacggatcatatgctgacggtggagtggtcctcagagtttggatgggagaaacctcatatcaagcctcttcagaacctgtcattgcaccctggctcatcagctttgcactatgcagtggaagtatttgacaaagaagagctcttagagtgtattcaacagcttgtgaaattggatcaagaatgggtcccatattcaacatctgctagtctgtatattcgtcctacattcattggaactgagccttctcttggagtcaagaagcctaccaaagccctgctctttgtactcttgagcccagtgggaccttatttttcaagtggaacctttaatccagtgtccctgtgggccaatcccaagtatgtaagagcctggaaaggtggaactggggactgcaagatgggagggaattacggctcatctctttttgcccaatgtgaagcagtagataatgggtgtcagcaggtcctgtggctctatggagaggaccatcagatcactgaagtgggaactatgaatctttttctttactggataaatgaagatggagaagaagaactggcaactcctccactagatggcatcattcttccaggagtgacaaggcggtgcattctggacctggcacatcagtggggtgaatttaaggtgtcagagagatacctcaccatggatgacttgacaacagccctggaggggaacagagtgagagagatgtttggctctggtacagcctgtgttgtttgcccagtttctgatatactgtacaaaggcgagacaatacacattccaactatggagaatggtcctaagctggcaagccgcatcttgagcaaattaactgatatccagtatggaagagaagagagcgactggacaattgtgctatcctgaatggaaaatagaggatacaatggaaaatagaggataccaactgtatgctactgggacagactgttgcatttgaattgtgatagatttctttggctacctgtgcataatgtagtttgtagtatcaatgtgttacaagagtgattgtttcttcatgccagagaaaatgaattgcaatcatcaaatggtgtttcataacttggtagtagtaacttaccttaccttacctagaaaaacattaatgtaagccatataacatgggattttcctcaatgattttagtgcctccttttgtacttcactcagatactaaatagtagtttattctttaatataagttacattctgctcctcaaacaaatgcaattttttgtgtgtgtttgaaagctaatttgagaaaatttcataggttacatttcctgcagcctatctttatccacagaaagtgttttcttttttttaaatcaagacttttaaaactggatttcctcccatcactgttttttgaaggtcctccaagtccgtgttaaggtaaatatctgttttcttcctgatgtcacagcctgagcatactctgtgcattaggaagacctgagtgcatttcccaccattgtcctttccacattatgttgtagctggctggctgtcaggcgactacaagactgagggtcttgtgccttatagatctttgtatcccccatggctgacatatagtaggtactcagtaaatggttttataatgaatcagtgaacattttgcttctatagaagtgtaccttctttgtttctatattatgaaacctctttattagaatttgtgattgattctgacagtgtatagatttaccttatattgtctttattttccatgagctactaagtcattagagatactctgaagcatagttagtttaggaaatcacttcatattgattgtattagaattatcttggaattgaagatatatccctagagcaggggaccccaacccccaggccatgggccacacagcaggaagaggtgagtggtgggccattgaggagcttcatctgtatttatggctacttcccatcactcgaattaccacctgaactccacctcttgtcagctcagtggcagcattagattctcataggagcacaaatcctattgtgaactctgcatgcaagggatctaggctatgcgctccttatgagaatctaatgcttgatgacctgaggtgtaacagtttcatcctgaaaccacccttcaccctgcagtctgtggaaaaattgtcttccacaaaactggtccctggtgccaaaaatgttggggaccactgctctagagagaggtcatgatatcataccaaccaaatggaaatgacaaatgttttatgtcaagtgttaattgcagaaataaatcttttttttttttttttggtagaaaacaaagaggcatactctgatttttatactctgtttttgcaggtgctcttttctttgaatggagatttgatgagcaagtggttaggatgcagggagagctactatgggtgatattttccttgtttaggagctgtgagttaaaattgtatcctttgtggtttatctaaggaaagtcaaatcttgacagaaaacatttttccttggaaggtcaactctcagacattgtattttggtttccctcagtcctcataacttccttcttgctgaacatattttattctcttttcagagaaggaaaataaaaaggattctaaaagtttgatgcattggaaaaatttccttgaggcatttagcaacacatagaaaatgggctttgattcttttccaaaacttttagccatagggtcttttatagacagggatagtaaaatgaaaattgagaaatataagatgaaaaggaatgataaaaatatcttttagggggcttttaattggtgatctgaaatcttgggagaagctgttcttttcaggcctgaggtgctcttgactgtcgcctgcgcactgtgtaccccgagcaacattctaagggtgtgctttcgccttggctaactcctttgacctcattcttcatatagtagtctaggaaaaagttgcaggtaatttaaactgtctagtggtacatagtaactaaatttctattcctatgagaaatgagaattatttatttgccatcaacacattttatactttgcatctccaaatttattgtggcgagacttgtccattgtgaaagttagagaacattatgtttgtatcatttctttcataaaacctcaagagcatttttaagcccttttcatcagacccagtgaaaactaaggatagatgtttaaaaactggaggtctcctgataaggagaacacaatccaccattgtcatttaagtaataagacaggaaattgaccttgacgctttcttgttaaatagatttaacaggaacatctgcacatcttttttccttgtgcactatttgtttaattgcagtggattaatacagcaagagtgccacattataactaggcaattatccattcttcaagacttagttattgtcacactaattgatcgtttaaggcataagatggtctagcattaggaacatgtgaagctaatctgctcaaaaagatcaacaaattaatattgttgctgatatttgcataattggctgcaattatttaatgtttaattgggttgatcaaatgagattcagcaattcacaagtgcattaatataaacagaactggtggcacttaaaatgataatgattaacttatattgcatgttctcttcctttcacttttttcagtttctacatttcagaccgagcttgtcagcttttttgaaaacacatcagtagaaaccaagattttaaaatgaagtgtcaagacaaaggcaaaacctgagcagttcctaaaaagatttgctgttagaaattttctttgtggcagtcatttattaaggattcaactcgtgatacaccaaaagaagagttgacttcagagatgtgttccatgctctctagcacaggaatgaataaatttataacacctgctttagcctttgttttcaaaagcacaaaggaaaagtgaaagggaaagagaaacaagtgactgagaagtcttgttaaggaatcaggttttttctacctggtaaacattctctattcttttctcaaaagattgctgtaagaaaaaatgtaagacaaaaaaaaaaaaaaaaaacaaacagaggcagaggcaggcagtagcaagaaagcagagcgtaacatcagctagatggtaacatgcaatgtcagctctcttgaagacatgggaaacctaagttacaccttgggttaaaattcttcaccatattagttttgttgcttcataaaatttacctaagcaagtggtcttgcttgcctcaaatccaagcagtcttgaacacttggaggcaattaatgagtatatcttagtcaaaagaattgttggagctttttattaaagctacagtttcagttctgcttttggggaattgtgctatgaaagcagctgccaaaataagctcatttattttcttcaatcccactcagtgctcagtcactatattctgtttcctttttttttttcaagttgcatatttggtttccccttatgattgggaaagatgaattttcagcagaaaacattgtttgttcactttcaaagagtgatagtttctaaaacatttagagcaataaatattcatcagaggtaccaagtaagccggcagaagagttaagggttagagaaatcccttatttcatgtcttgactctaaaattatcaaagtacttttccttgtaatgtggatttcttcttatgcggatatgcaaaaacttcagttatacgtagtaatgctagcaggtaattttagtagacattttataacaactgtcactttgtttcgccacatgtagagtttgttcagctattttccagatatctccccacaaaaggaggcaaagggtaccagcttttcaatgagcattacctattacttggcaaagatgatgaagactctattaatagttcatttgataaatgttgacataaccaacaatagagattaggaagttagttttaagaaatcaatggcatatagacattaccctcatggagtttgtattctactacttgaactgattgtagctataaaagcatagttagatagctgaatagttagatcataagcaaagaaggccagaacacatctcttatcaagaaatcaatgaatagtttatctcatttttaaagcaactttatccttctttaattccttcctttcttctagtgcaaaactacttaataaggttggtgtttaggttagtgttcacaccattcctcatctggtgtgaattaccttctctttctttactatttactaccaacctagtacatgtgttgactgaattcttttcaaacaatgttgagttatcatggtgcacctaataaattaacaccacagattacagcatccttgctgattttctcagcaaagccagattagatggaaataaacaaagaaaatgatcctagagtgaatttttctagaaaatatctattatgaaccatgctgtttaaagtattagcttgaaggtgatggatccagctattcagaaaataactttcatataaccatgattttgcacagtatgaggtcttaaatgtgtggaaagagataaattttttatcattaccacaaaccccttttaaagattcaaaggtggaagaaagtgatttattttttctcttcagcatacatatataaaagacttgtcagatgtttaatttggggaggttgataatgaaacatatcaacagagtatagtagttatagtagtgtttgtgggtaaataatttcctggggtcagacatatataaacatatttgcttcaaaatgataaaggcatgaaatcagtcttaaaaattgaaatgggggtgatgggggagaaaaagaagaacaaatttgaagtgccctttcaaatctgctggatacaagtattgaagttttaagtcatcttattctgtctgaaagtgtatttttcattctacaatagacccaatcaacaagacgtataacttgagttgcatgatgttcagtttatgtaatctactgttgggatggtaagaattgatgtaggctgtggtgtaagaatgaattaaaatatagtttcactggcttttctctacatatccactatcacaatggctaggtttcctgttgctcactattggattctggagaaaaatttaatgaaagatgatatcagaggaagaataagtggaggtagagaagaaaggaatgatagaggaggggaaaaaaacaaaacatatttttgtgttatccaaaggagctttttccttattctgtcaagcattgagatcttcttcagctttcaatgtagttgctaaatacaaataatgctactaggtagtgactaaatatagcaaacacttcatcagatattagaattaggtcacactattgaggttataatctgaaggttgtgttacatagaaaccactttagattattatcaacttggactaggctttattttataatagcatagtaagtaatatctattgtgtcatttcttcaaccattttattctaagatccatgaagcttcttgaggccaaataaaataataagtttagacaagaagtagattgtgacttttttcccttagagatactatttactatctcctatcctgataggtggaaggtttactgaattggaaattggttgactattagtttttaactaaaatgtgcaataacacattgcagtttcctcaaactagtttcctatgatcattaaactcattctcagggttaagaaaggaatgtaaatttctgcctcaatttgtacttcatcaataagtttttgaagagtgcagatttttagtcaggtcttaaaaataaactcacaaatctggatgcatttctaaattctgcaaatgtttcctggggtgacttaacaaggaataatcccacaatatacctagctacctaatacatggagctggggctcaacccactgtttttaaggatttgcgcttacttgtggctgaggaaaaataagtagttcgaggaagtagtttttaaatgtgagcttatagatagaaacagaatatcaacttaattatgaaattgttagaacctgttctcttgtatctgaatctgattgcaattactattgtactgatagactccagccattgcaagtctcagatatcttagctgtgtagtgattcttgaaattctttttaagaaaaattgagtagaaagaaataaaccctttgtaaatgaggcttggcttttgtgaaagatcatccgcaggctatgttaaaaggattttagctcactaaaagtgtaataatggaaatgtggaaaatatcgtaggtaaaggaaactacctcatgctctgaaggttttgtagaagcacaattaaacatctaaaatggctttgttacaccagagccatctggtgtgaagaactctatatttgtatgttgagagggcatggaataattgtattttgctggcaatagacacattctttattatttgcagattcctcatcaaatctgtaattatgcacagtttctgttatcaataaaacaaaagaatcctgtttgtgtggtttcatgaaatcagcattgttgaatgcatgaagtaataatgctaaattaacatttttatgatgtctcaaggtttctggtcaagggaagtaaatgtaggatagtatttttacaccaaaatgacacagagagaattgagcacaccagaaagaccagaaaccacaccactggatagagattcaatatgtttctttttcaaacatttggacaagaaaaaaatgggcatttaaaaattcttccttcccctggttatggatttatctgtagtaaaacttagctttgtcgtttgagatttgcacagaatggggggagtagattacctcttcccattcattgtcataatggatctacatcactgataaacaccatacttctatatgtggttaactagctttagaataaaagacactttaaaaagtaaaaggcctagagatcttcaatgaagtgtcccttttagccaaaccaggccttgcagaaattgtcctcaaaagcaccaagggagaacaaagccaagtgcagaattacccaagggtcacacattttgtattcattttcttataatttgcccaaatgatttgaagtacagcaaaacctgaagttttgcaaagagttactgtaaactgaacttaaaaatgtcagagtgctcggtgacccacttctccagaccctgtcaacctgtgaaaatattggcctttattcagatctctcaagaagttacgcgcagagtttggaaggtctaggcaaaggttattagtcaagtgttcttacagtgtcaacgctcaattcccacaagcgtgagaaagagagacctgtcattcctgagggtgatgacatacatttactggagcttatataatttatcagataagacagcagtttccttcagggtagaaagtgtgttttctacattgatttagtacaaaacaaaaagaaaaggggatatttcaaattttataattatttttctgctaagctgattcagtgtgatttaagcatatttttcaaatcatgaatctgattccatatacatatgtgccttatattgtgataatttatttttaagtgaaatatgctatcatagcctgactttatgtatagtggtgacaacttgcaggatcgcatttctgtaaccaaacggccgacagctgaggtgtagatgcttcctatggtctgtagaataatcactgggcttgttctctagctatgtctgtatgcaatcgcgacagtgttgatcaaaacccatgatcattctctccaaaggtctttgtcactaagccactagggattctgaaaagttcgtgagctgaaacaaataaattgagttggaagattaaaaaaaaaa

SEQ ID NO: 25 - protein, 76 aa, Homo sapiens
  MISC_FEATURE: ADIRF ACCESSION   NM_006829
MASKGLQDLKQQVEGTAQEAVSAAGAAAQQVVDQATEAGQKAMDQLAKTTQETIDKTANQASDTFSGIGKKFGLLK

SEQ ID NO: 26 - DNA, 672 bp, Homo sapiens
  misc_feature: cDNA  ADIRF
caggccagccctggggcgccttaaaaaccggagctggcgcttggcatcgccactctgggcaggatccaacgtcgctccagctgctcttgacgactccacagataccccgaagccatggcaagcaagggcttgcaggacctgaagcaacaggtggaggggaccgcccaggaagccgtgtcagcggccggagcggcagctcagcaagtggtggaccaggccacagaggcggggcagaaagccatggaccagctggccaagaccacccaggaaaccatcgacaagactgctaaccaggcctctgacaccttctctgggattgggaaaaaattcggcctcctgaaatgacagcagggagacttgggtcggcctcctgaaatgacagcagggagacttgggtgaccccccttccaggcgccatctagcacagcctggccctgatctccgggcagccaccacctcctcggtctgccccctcattaaaattcacgttcccaccctgtgtccacttcatgattcctcgcaagctgggcccagtcctctcatcccaagagcagagccaccgtagccggagtcctagcctcccaaattcggaaatccaatccaacggtctcaggaatgttttccatcccgccacgcgcctcccgaagctcccagaccggaggctcagccccc

SEQ ID NO: 27 - protein, 495 aa, Homo sapiens
  MISC_FEATURE: CRNN ACCESSION   NM_016190
MPQLLQNINGIIEAFRRYARTEGNCTALTRGELKRLLEQEFADVIVKPHDPATVDEVLRLLDEDHTGTVEFKEFLVLVFKVAQACFKTLSESAEGACGSQESGSLHSGASQELGEGQRSGTEVGRAGKGQHYEGSSHRQSQQGSRGQNRPGVQTQGQATGSAWVSSYDRQAESQSQERISPQIQLSGQTEQTQKAGEGKRNQTTEMRPERQPQTREQDRAHQTGETVTGSGTQTQAGATQTVEQDSSHQTGRTSKQTQEATNDQNRGTETHGQGRSQTSQAVTGGHAQIQAGTHTQTPTQTVEQDSSHQTGSTSTQTQESTNGQNRGTEIHGQGRSQTSQAVTGGHTQIQAGSHTETVEQDRSQTVSHGGAREQGQTQTQPGSGQRWMQVSNPEAGETVPGGQAQTGASTESGRQEWSSTHPRRCVTEGQGDRQPTVVGEEWVDDHSRETVILRLDQGNLHTSVSSAQGQDAAQSEEKRGITARELYSYLRSTKP

SEQ ID NO: 28 - DNA, 1913 bp, Homo sapiens
  misc_feature: cDNA  CRNN
actgacctggtactcctcacaccacttaacagccacttgtttcatcccacctgggcattaggttgacttcaaagatgcctcagttactgcaaaacattaatgggatcatcgaggccttcaggcgctatgcaaggacggagggcaactgcacagcgctcacccgaggggagctgaaaagactcttggagcaagagtttgccgatgtgattgtgaaaccccacgatccagcaactgtggatgaggtcctgcgtctgctggatgaagaccacacagggactgtggaattcaaggaattcctggtcttagtgtttaaagttgcccaggcctgtttcaagacactgagcgagagtgctgagggagcctgcggctctcaagagtctggaagcctccactctggggcctcgcaggagctgggcgaaggacagagaagtggcactgaagtgggaagggcggggaaagggcagcattatgaggggagcagccacagacagagccagcagggttccagagggcagaacaggcctggggttcagacccagggtcaggccactggctctgcgtgggtcagcagctatgacaggcaagctgagtcccagagccaggaaagaataagcccgcagatacaactctctgggcagacagagcagacccagaaagctggagaaggcaagaggaatcagacaacagagatgaggccagagagacagccacagaccagggaacaggacagagcccaccagacaggtgagactgtgactggatctggaactcagacccaggcaggtgccacccagactgtggagcaggacagcagccaccagacaggaagaaccagcaagcagacacaggaggccaccaatgaccagaacagagggactgagacccacggtcaaggcaggagccagaccagccaggctgtgacaggaggacatgctcagatacaggcagggacacacacccagacacccacccagaccgtggagcaggacagcagccaccagacaggaagcaccagcacccagacacaggagtccaccaatggccagaacagagggactgagatccacggtcaaggcaggagccagaccagccaggctgtgacaggaggacacactcagatacaggcagggtcacacaccgagactgtggagcaggacagaagccaaactgtaagccacggaggggctagagaacagggacagacccagacgcagccaggcagtggtcaaagatggatgcaagtgagcaaccctgaggcaggagagacagtaccgggaggacaggcccagactggggcaagcactgagtcaggaaggcaggagtggagcagcactcacccaaggcgctgtgtgacagaagggcagggagacagacagcccacagtggttggtgaggaatgggttgatgaccactcaagggagacagtgatcctcaggctggaccagggcaacttgcataccagtgtttcctcagcacagggccaggatgcagcccagtcagaagagaagcgaggcatcacagctagagagctgtattcctacttgagaagcaccaagccatgacttccccgactccaatgtccagtactggaagaagacagctggagagagtttggcttgtcctgcatggccaatccagtgggtgcatccctggacatcagctcttcattatgcagcttcccttttaggtctttctcaatgagataatttctgcaaggagctttctatcctgaactcttctttcttacctgctttgcggtgcagaccctctcaggagcaggaagactcagagcaagtcacccctttgtactgaattgtcctcatcttgtggggggtttcaggactatttttatctctgacatctctctattgccccatctaccctaatgcatcaataaaaccttaagccactgg

SEQ ID NO: 29 - protein, 2045 aa, Homo sapiens
  MISC_FEATURE: AGRN ACCESSION   NM_198576
MAGRSHPGPLRPLLPLLVVAACVLPGAGGTCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKPGTHFTPVPPTPPDACRGMLCGFGAVCEPNAEGPGRASCVCKKSPCPSVVAPVCGSDASTYSNECELQRAQCSQQRRIRLLSRGPCGSRDPCSNVTCSFGSTCARSADGLTASCLCPATCRGAPEGTVCGSDGADYPGECQLLRRACARQENVFKKFDGPCDPCQGALPDPSRSCRVNPRTRRPEMLLRPESCPARQAPVCGDDGVTYENDCVMGRSGAARGLLLQKVRSGQCQGRDQCPEPCRFNAVCLSRRGRPRCSCDRVTCDGAYRPVCAQDGRTYDSDCWRQQAECRQQRAIPSKHQGPCDQAPSPCLGVQCAFGATCAVKNGQAACECLQACSSLYDPVCGSDGVTYGSACELEATACTLGREIQVARKGPCDRCGQCRFGALCEAETGRCVCPSECVALAQPVCGSDGHTYPSECMLHVHACTHQISLHVASAGPCETCGDAVCAFGAVCSAGQCVCPRCEHPPPGPVCGSDGVTYGSACELREAACLQQTQIEEARAGPCEQAECGSGGSGSGEDGDCEQELCRQRGGIWDEDSEDGPCVCDFSCQSVPGSPVCGSDGVTYSTECELKKARCESQRGLYVAAQGACRGPTFAPLPPVAPLHCAQTPYGCCQDNITAARGVGLAGCPSACQCNPHGSYGGTCDPATGQCSCRPGVGGLRCDRCEPGFWNFRGIVTDGRSGCTPCSCDPQGAVRDDCEQMTGLCSCKPGVAGPKCGQCPDGRALGPAGCEADASAPATCAEMRCEFGARCVEESGSAHCVCPMLTCPEANATKVCGSDGVTYGNECQLKTIACRQGLQISIQSLGPCQEAVAPSTHPTSASVTVTTPGLLLSQALPAPPGALPLAPSSTAHSQTTPPPSSRPRTTASVPRTTVWPVLTVPPTAPSPAPSLVASAFGESGSTDGSSDEELSGDQEASGGGSGGLEPLEGSSVATPGPPVERASCYNSALGCCSDGKTPSLDAEGSNCPATKVFQGVLELEGVEGQELFYTPEMADPKSELFGETARSIESTLDDLFRNSDVKKDFRSVRLRDLGPGKSVRAIVDVHFDPTTAFRAPDVARALLRQIQVSRRRSLGVRRPLQEHVRFMDFDWFPAFITGATSGAIAAGATARATTASRLPSSAVTPRAPHPSHTSQPVAKTTAAPTTRRPPTTAPSRVPGRRPPAPQQPPKPCDSQPCFHGGTCQDWALGGGFTCSCPAGRGGAVCEKVLGAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 30 - DNA, 7343 bp, Homo sapiens
  misc_feature: cDNA AGRN
cccgtccccggcgcggcccgcgcgctcctccgccgcctctcgcctgcgccatggccggccggtcccacccgggcccgctgcggccgctgctgccgctccttgtggtggccgcgtgcgtcctgcccggagccggcgggacatgcccggagcgcgcgctggagcggcgcgaggaggaggcgaacgtggtgctcaccgggacggtggaggagatcctcaacgtggacccggtgcagcacacgtactcctgcaaggttcgggtctggcggtacttgaagggcaaagacctggtggcccgggagagcctgctggacggcggcaacaaggtggtgatcagcggctttggagaccccctcatctgtgacaaccaggtgtccactggggacaccaggatcttctttgtgaaccctgcacccccatacctgtggccagcccacaagaacgagctgatgctcaactccagcctcatgcggatcaccctgcggaacctggaggaggtggagttctgtgtggaagataaacccgggacccacttcactccagtgcctccgacgcctcctgatgcgtgccggggaatgctgtgcggcttcggcgccgtgtgcgagcccaacgcggaggggccgggccgggcgtcctgcgtctgcaagaagagcccgtgccccagcgtggtggcgcctgtgtgtgggtcggacgcctccacctacagcaacgaatgcgagctgcagcgggcgcagtgcagccagcagcgccgcatccgcctgctcagccgcgggccgtgcggctcgcgggacccctgctccaacgtgacctgcagcttcggcagcacctgtgcgcgctcggccgacgggctgacggcctcgtgcctgtgccccgcgacctgccgtggcgcccccgaggggaccgtctgcggcagcgacggcgccgactaccccggcgagtgccagctcctgcgccgcgcctgcgcccgccaggagaatgtcttcaagaagttcgacggcccttgtgacccctgtcagggcgccctccctgacccgagccgcagctgccgtgtgaacccgcgcacgcggcgccctgagatgctcctacggcccgagagctgccctgcccggcaggcgccagtgtgtggggacgacggagtcacctacgaaaacgactgtgtcatgggccgatcgggggccgcccggggtctcctcctgcagaaagtgcgctccggccagtgccagggtcgagaccagtgcccggagccctgccggttcaatgccgtgtgcctgtcccgccgtggccgtccccgctgctcctgcgaccgcgtcacctgtgacggggcctacaggcccgtgtgtgcccaggacgggcgcacgtatgacagtgattgctggcggcagcaggctgagtgccggcagcagcgtgccatccccagcaagcaccagggcccgtgtgaccaggccccgtccccatgcctcggggtgcagtgtgcatttggggcgacgtgtgctgtgaagaacgggcaggcagcgtgtgaatgcctgcaggcgtgctcgagcctctacgatcctgtgtgcggcagcgacggcgtcacatacggcagcgcgtgcgagctggaggccacggcctgtaccctcgggcgggagatccaggtggcgcgcaaaggaccctgtgaccgctgcgggcagtgccgctttggagccctgtgcgaggccgagaccgggcgctgcgtgtgcccctctgaatgcgtggctttggcccagcccgtgtgtggctccgacgggcacacgtaccccagcgagtgcatgctgcacgtgcacgcctgcacacaccagatcagcctgcacgtggcctcagctggaccctgtgagacctgtggagatgccgtgtgtgcttttggggctgtgtgctccgcagggcagtgtgtgtgtccccggtgtgagcaccccccgcccggccccgtgtgtggcagcgacggtgtcacctacggcagtgcctgcgagctacgggaagccgcctgcctccagcagacacagatcgaggaggcccgggcagggccgtgcgagcaggccgagtgcggttccggaggctctggctctggggaggacggtgactgtgagcaggagctgtgccggcagcgcggtggcatctgggacgaggactcggaggacgggccgtgtgtctgtgacttcagctgccagagtgtcccaggcagcccggtgtgcggctcagatggggtcacctacagcaccgagtgtgagctgaagaaggccaggtgtgagtcacagcgagggctctacgtagcggcccagggagcctgccgaggccccaccttcgccccgctgccgcctgtggcccccttacactgtgcccagacgccctacggctgctgccaggacaatatcaccgcagcccggggcgtgggcctggctggctgccccagtgcctgccagtgcaacccccatggctcttacggcggcacctgtgacccagccacaggccagtgctcctgccgcccaggtgtggggggcctcaggtgtgaccgctgtgagcctggcttctggaactttcgaggcatcgtcaccgatggccggagtggctgtacaccctgcagctgtgatccccaaggcgccgtgcgggatgactgtgagcagatgacggggctgtgctcgtgtaagcccggggtggctggacccaagtgtgggcagtgtccagacggccgtgccctgggccccgcgggctgtgaagctgacgcttctgcgcctgcgacctgtgcggagatgcgctgtgagttcggtgcgcggtgcgtggaggagtctggctcagcccactgtgtctgcccgatgctcacctgtccagaggccaacgctaccaaggtctgtgggtcagatggagtcacatacggcaacgagtgtcagctgaagaccatcgcctgccgccagggcctgcaaatctctatccagagcctgggcccgtgccaggaggctgttgctcccagcactcacccgacatctgcctccgtgactgtgaccaccccagggctcctcctgagccaggcactgccggccccccccggcgccctccccctggctcccagcagtaccgcacacagccagaccacccctccgccctcatcacgacctcggaccactgccagcgtccccaggaccaccgtgtggcccgtgctgacggtgccccccacggcaccctcccctgcacccagcctggtggcgtccgcctttggtgaatctggcagcactgatggaagcagcgatgaggaactgagcggggaccaggaggccagtgggggtggctctggggggctcgagcccttggagggcagcagcgtggccacccctgggccacctgtcgagagggcttcctgctacaactccgcgttgggctgctgctctgatgggaagacgccctcgctggacgcagagggctccaactgccccgccaccaaggtgttccagggcgtcctggagctggagggcgtcgagggccaggagctgttctacacgcccgagatggctgaccccaagtcagaactgttcggggagacagccaggagcattgagagcaccctggacgacctcttccggaattcagacgtcaagaaggattttcggagtgtccgcttgcgggacctggggcccggcaaatccgtccgcgccattgtggatgtgcactttgaccccaccacagccttcagggcacccgacgtggcccgggccctgctccggcagatccaggtgtccaggcgccggtccttgggggtgaggcggccgctgcaggagcacgtgcgatttatggactttgactggtttcctgcgtttatcacgggggccacgtcaggagccattgctgcgggagccacggccagagccaccactgcatcgcgcctgccgtcctctgctgtgacccctcgggccccgcaccccagtcacacaagccagcccgttgccaagaccacggcagcccccaccacacgtcggccccccaccactgcccccagccgtgtgcccggacgtcggcccccggccccccagcagcctccaaagccctgtgactcacagccctgcttccacggggggacctgccaggactgggcattgggcgggggcttcacctgcagctgcccggcaggcaggggaggcgccgtctgtgagaaggtgcttggcgcccctgtgccggccttcgagggccgctccttcctggccttccccactctccgcgcctaccacacgctgcgcctggcactggaattccgggcgctggagcctcaggggctgctgctgtacaatggcaacgcccggggcaaggacttcctggcattggcgctgctagatggccgcgtgcagctcaggtttgacacaggttcggggccggcggtgctgaccagtgccgtgccggtagagccgggccagtggcaccgcctggagctgtcccggcactggcgccggggcaccctctcggtggatggtgagacccctgttctgggcgagagtcccagtggcaccgacggcctcaacctggacacagacctctttgtgggcggcgtacccgaggaccaggctgccgtggcgctggagcggaccttcgtgggcgccggcctgagggggtgcatccgtttgctggacgtcaacaaccagcgcctggagcttggcattgggccgggggctgccacccgaggctctggcgtgggcgagtgcggggaccacccctgcctgcccaacccctgccatggcggggccccatgccagaacctggaggctggaaggttccattgccagtgcccgcccggccgcgtcggaccaacctgtgccgatgagaagagcccctgccagcccaacccctgccatggggcggcgccctgccgtgtgctgcccgagggtggtgctcagtgcgagtgccccctggggcgtgagggcaccttctgccagacagcctcggggcaggacggctctgggcccttcctggctgacttcaacggcttctcccacctggagctgagaggcctgcacacctttgcacgggacctgggggagaagatggcgctggaggtcgtgttcctggcacgaggccccagcggcctcctgctctacaacgggcagaagacggacggcaagggggacttcgtgtcgctggcactgcgggaccgccgcctggagttccgctacgacctgggcaagggggcagcggtcatcaggagcagggagccagtcaccctgggagcctggaccagggtctcactggagcgaaacggccgcaagggtgccctgcgtgtgggcgacggcccccgtgtgttgggggagtccccggttccgcacaccgtcctcaacctgaaggagccgctctacgtagggggcgctcccgacttcagcaagctggcccgtgctgctgccgtgtcctctggcttcgacggtgccatccagctggtctccctcggaggccgccagctgctgaccccggagcacgtgctgcggcaggtggacgtcacgtcctttgcaggtcacccctgcacccgggcctcaggccacccctgcctcaatggggcctcctgcgtcccgagggaggctgcctatgtgtgcctgtgtcccgggggattctcaggaccgcactgcgagaaggggctggtggagaagtcagcgggggacgtggataccttggcctttgacgggcggacctttgtcgagtacctcaacgctgtgaccgagagcgagaaggcactgcagagcaaccactttgaactgagcctgcgcactgaggccacgcaggggctggtgctctggagtggcaaggccacggagcgggcagactatgtggcactggccattgtggacgggcacctgcaactgagctacaacctgggctcccagcccgtggtgctgcgttccaccgtgcccgtcaacaccaaccgctggttgcgggtcgtggcacatagggagcagagggaaggttccctgcaggtgggcaatgaggcccctgtgaccggctcctccccgctgggcgccacgcagctggacactgatggagccctgtggcttgggggcctgccggagctgcccgtgggcccagcactgcccaaggcctacggcacaggctttgtgggctgcttgcgggacgtggtggtgggccggcacccgctgcacctgctggaggacgccgtcaccaagccagagctgcggccctgccccaccccatgagctggcaccagagccccgcgcccgctgtaattattttctatttttgtaaacttgttgctttttgatatgattttcttgcctgagtgttggccggagggactgctggcccggcctcccttccgtccaggcagccgtgctgcagacagacctagtgccgagggatggacaggcgaggtggcagcgtggagggctcggcgtggatggcagcctcaggacacacacccctgcctcaaggtgctgagcccccgccttgcactgcgcctgccccacggtgtccccgccgggaagcagccccggctcctgaatcaccctcgctccgtcaggcgggactcgtgtcccagagaggaaggggctgctgaggtctgatggggcccttcctccgggtgaccccacagggcctttccaagcccccatttgagctgctccttcctgtgtgtgctctgggccctgcctcggcctcctgcgccaatactgtgacttccaaacaatgttactgctgggcacagctctgcgttgctcccgtgctgcctgcgccagccccaggctgctgaggagcagaggccagaccagggccgatctgggtgtcctgaccctcagctggccctgcccagccaccctggacgtgaccgtatccctctgccacaccccaggccctgcgaggggctatcgagaggagctcactgtgggatggggttgacctctgccgcctgcctgggtatctgggcctggccatggctgtgttcttcatgtgttgattttatttgacccctggagtggtgggtctcatctttcccatctcgcctgagagcggctgagggctgcctcactgcaaatcctccccacagcgtcagtgaaagtcgtccttgtctcagaatgaccaggggccagccagtgtctgaccaaggtcaaggggcaggtgcagaggtggcagggatggctccgaagccagaaatgccttaaactgcaacgtcccgtcccttccccacccccatcccatccccacccccagccccagcccagtcctcctaggagcaggacccgatgaagcgggcggcggtggggctgggtgccgtgttactaactctagtatgtttctgtgtcaatcgctgtgaaataaagtctgaaaactttaaaagcaaaaaaaaaaaaaaaaaa

SEQ ID NO: 31 - protein, 335 aa, Homo sapiens
  MISC_FEATURE: ADH1B ACCESSION   NM_001286650
MVAVGICHTDDHVVSGNLVTPLPVILGHEAAGIVESVGEGVTTVKPGDKVIPLFTPQCGKCRVCKNPESNYCLKNDLGNPRGTLQDGTRRFTCRGKPIHHFLGTSTFSQYTVVDENAVAKIDAASPLEKVCLIGCGFSTGYGSAVNVAKVTPGSTCAVFGLGGVGLSAVMGCKAAGAARIIAVDINKDKFAKAKELGATECINPQDYKKPIQEVLKEMTDGGVDFSFEVIGRLDTMMASLLCCHEACGTSVIVGVPPASQNLSINPMLLLTGRTWKGAVYGGFKSKEGIPKLVADFMAKKFSLDALITHVLPFEKINEGFDLLHSGKSIRTVLTF

SEQ ID NO: 32 - DNA, 2829 bp, Homo sapiens
  misc_feature: cDNA ADH1B
acaagcaaacaaaataaatatctgtgcaatatatctgctttatgcactcaagcagagaagaaatccacaaagactcacagtctgctggtgggcagagaagacagaaacgacatgagcacagcaggaaaagaaagttgtctgacagaagtttgatgagaggaatttgactcagaagactgaaatatcctttaaccttactataaaatatcttacaaaatactcattgatttcacagcattagaatcatcaaggtaatcaaatgcaaagcagctgtgctatgggaggtaaagaaacccttttccattgaggatgtggaggttgcacctcctaaggcttatgaagttcgcattaagatggtggctgtaggaatctgtcacacagatgaccacgtggttagtggcaacctggtgaccccccttcctgtgattttaggccatgaggcagccggcatcgtggagagtgttggagaaggggtgactacagtcaaaccaggtgataaagtcatcccgctctttactcctcagtgtggaaaatgcagagtttgtaaaaacccggagagcaactactgcttgaaaaatgatctaggcaatcctcgggggaccctgcaggatggcaccaggaggttcacctgcagggggaagcccattcaccacttccttggcaccagcaccttctcccagtacacggtggtggatgagaatgcagtggccaaaattgatgcagcctcgcccctggagaaagtctgcctcattggctgtggattctcgactggttatgggtctgcagttaacgttgccaaggtcaccccaggctctacctgtgctgtgtttggcctgggaggggtcggcctatctgctgttatgggctgtaaagcagctggagcagccagaatcattgcggtggacatcaacaaggacaaatttgcaaaggccaaagagttgggtgccactgaatgcatcaaccctcaagactacaagaaacccattcaggaagtgctaaaggaaatgactgatggaggtgtggatttttcgtttgaagtcatcggtcggcttgacaccatgatggcttccctgttatgttgtcatgaggcatgtggcacaagcgtcatcgtaggggtacctcctgcttcccagaacctctcaataaaccctatgctgctactgactggacgcacctggaagggggctgtttatggtggctttaagagtaaagaaggtatcccaaaacttgtggctgattttatggctaagaagttttcactggatgcgttaataacccatgttttaccttttgaaaaaataaatgaaggatttgacctgcttcactctgggaaaagtatccgtaccgtcctgacgttttgaggcaatagagatgccttcccctgtagcagtcttcagcctcctctaccctacaagatctggagcaacagctaggaaatatcattaattcagctcttcagagatgttatcaataaattacacatgggggctttccaaagaaatggaaattgatgggaaattatttttcaggaaaatttaaaattcaagtgagaagtaaataaagtgttgaacatcagctggggaattgaagccaacaaaccttccttcttaaccattctactgtgtcacctttgccattgaggaaaaatattcctgtgacttcttgcatttttggtatcttcataatctttagtcatcgaatcccagtggaggggacccttttacttgccctgaacatacacatgctgggccattgtgattgaagtcttctaactctgtctcagttttcactgtcgacattttcctttttctaataaaaatgtaccaaatccctggggtaaaagctagggtaaggtaaaggatagactcacatttacaagtagtgaaggtccaagagttctaaatacaggaaatttcttaggaactcaaataaaatgccccacattttactacagtaaatggcagtgtttttatgacttttatactatttctttatggtcgatatacaattgattttttaaaataatagcagatttcttgcttcatatgacaaagcctcaattactaattgtaaaaactgaactattcccagaatcatgttcaaaaaatctgtaatttttgctgatgaaagtgcttcattgactaaacagtattagtttgtggctataaatgattatttagatgatgactgaaaatgtgtataaagtaattaaaagtaatatggtggctttaagtgtagagatgggatggcaaatgctgtgaatgcagaatgtaaaattggtaactaagaaatggcacaaacaccttaagcaatatattttcctagtagatatatatatacacatacatatatacacatatacaaatgtatatttttgcaaaattgttttcaatctagaacttttctattaactaccatgtcttaaaatcaagtctataatcctagcattagtttaatattttgaatatgtaaagacctgtgttaatgctttgttaatgcttttcccactctcatttgttaatgctttcccactctcaggggaaggatttgcattttgagctttatctctaaatgtgacatgcaaagattattcctggtaaaggaggtagctgtctccaaaaatgctattgttgcaatatctacattctatttcatattatgaaagaccttagacataaagtaaaatagtttatcatttactgtgtgatcttcagtaagtctctcaggctctctgagcttgttcatcctttgttttgaaaaaattactcaaccaatccattacagcttaaccaagattaaatgggatgatgttaaaaaaaaaaaaaaaaa

SEQ ID NO: 33 - protein, 882 aa, Homo sapiens
  MISC_FEATURE: CDH1 ACCESSION   NM_004360
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 34 - DNA, 4815 bp, Homo sapiens
  misc_feature: cDNA CDH1
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccat

SEQ ID NO: 35 - protein, 373 aa, Homo sapiens
  MISC_FEATURE: GLUL ACCESSION   NM_002065
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 36 - DNA, 8337 bp, Homo sapiens
  misc_feature: cDNA GLUL
gtaaaactattccccgtgaaggcggcagggcagaggtccagggcgggctttgctgggagcctcgggaccccgggttgggggccgtggggcggcacctggcgagctggcgggtgggcggcgagccgaggcttcccggcctggcggcaactcgcccctctgccctcagccctcccggctccgctcccttcccccacgccgccctgcccctcccccacgcccctttctctttctttctttctttcccagttcgcttgcccccaccccagcggcgcccgccgggctcctcgcccaatggccgcggggcccgggaccgcatcagctgatcggcccgggctcctggccgctgggagccaatcagggcaccgggggcggccccgggccgcggataaagggtgcggggctgctggcggctctgcagagtcgagagtgggagaagagcggagcgtgtgagcagtactgcggcctcctctcctctcctaacctcgctctcgcggcctagctttacccgcccgcctgctcggcgaccagcggggatcctcccccagccgcaagtccacgaagaaagcaacgaatgaaaattatgaagacaacgagaagtcagactcctccgggtcgcgctccagctgcttcggcttcgtcgcctactctgtgaactccggggagagatctcgagtcaagattaagaccttaacccaccaacctgcctgttcggacaccccccgggccggccgctgtctgtccccttctccatcgccctctcccagaaagctccggtgcttggaccagctagagtctgagaaagaggagaggcgcgaacgccactccaaaaagagaagggttaaagagggcaaccctaacgatacgcttgactttctgtggctgggaacaccttccaccatgaccacctcagcaagttcccacttaaataaaggcatcaagcaggtgtacatgtccctgcctcagggtgagaaagtccaggccatgtatatctggatcgatggtactggagaaggactgcgctgcaagacccggaccctggacagtgagcccaagtgtgtggaagagttgcctgagtggaatttcgatggctctagtactttacagtctgagggttccaacagtgacatgtatctcgtgcctgctgccatgtttcgggaccccttccgtaaggaccctaacaagctggtgttatgtgaagttttcaagtacaatcgaaggcctgcagagaccaatttgaggcacacctgtaaacggataatggacatggtgagcaaccagcacccctggtttggcatggagcaggagtataccctcatggggacagatgggcacccctttggttggccttccaacggcttcccagggccccagggtccatattactgtggtgtgggagcagacagagcctatggcagggacatcgtggaggcccattaccgggcctgcttgtatgctggagtcaagattgcggggactaatgccgaggtcatgcctgcccagtgggaatttcagattggaccttgtgaaggaatcagcatgggagatcatctctgggtggcccgtttcatcttgcatcgtgtgtgtgaagactttggagtgatagcaacctttgatcctaagcccattcctgggaactggaatggtgcaggctgccataccaacttcagcaccaaggccatgcgggaggagaatggtctgaagtacatcgaggaggccattgagaaactaagcaagcggcaccagtaccacatccgtgcctatgatcccaagggaggcctggacaatgcccgacgtctaactggattccatgaaacctccaacatcaacgacttttctgctggtgtagccaatcgtagcgccagcatacgcattccccggactgttggccaggagaagaagggttactttgaagatcgtcgcccctctgccaactgcgaccccttttcggtgacagaagccctcatccgcacgtgtcttctcaatgaaaccggcgatgagcccttccagtacaaaaattaagtggactagacctccagctgttgagcccctcctagttcttcatcccactccaactcttccccctctcccagttgtcccgattgtaactcaaagggtggaatatcaaggtcgtttttttcattccatgtgcccagttaatcttgctttctttgtttggctgggatagaggggtcaagttattaatttcttcacacctaccctcctttttttccctatcactgaagctttttagtgcattagtggggaggagggtggggagacataaccactgcttccatttaatggggtgcacctgtccaataggcgtagctatccggacagagcacgtttgcagaagggggtctcttcttccaggtagctgaaaggggaagacctgacgtactctggttaggttaggacttgccctcgtggtggaaacttttcttaaaaagttataaccaacttttctattaaaagtgggaattaggagagaaggtaggggttgggaatcagagagaatggctttggtctcttgcttgtgggactagcctggcttgggactaaatgccctgctctgaacacgaagcttagtataaactgatggatatccctaccttgaaagaagaaaaggttcttactgcttggtccttgatttatcacacaaagcagaatagtatttttatatttaaatgtaaagacaaaaaactatatgtatggttttgtggattatgtgtgttttgctaaaggaaaaaaccatccaggtcacggggcaccaaatttgagacaaatagtcggattagaaataaagcatctcattttgagtagagagcaagggaagtggttcttagatggtgatctgggattaggccctcaagacccttttgggtttctgccctgcccaccctctggagaaggtgggcactggattagttaacagacaacacgttactagcagtcacttgatctccgtggctttggtttaaaagacacacttgtccacataggtttagagataagagttggctggtcaacttgagcatgttactgacagagggggtattggggttattttctggtaggaatagcatgtcactaaagcaggccttttgatattaaattttttaaaaagcaaaattatagaagtttagattttaatcaaatttgtagggtttctaggtaatttttacagaattgcttgtttgcttcaactgtctcctacctctgctcttggaggagatggggacagggctggagtcaaaacacttgtaattttgtatcttgatgtctttgttaagactgctgaagaattattttttttcttttataataaggaataaaccccacctttattccttcatttcatctaccattttctggttcttgtgttggctgtggcaggccagctgtggttttcttttgccatgacaacttctaattgccatgtacagtatgttcaaagtcaaataactcctcattgtaaacaaactgtgtaactgcccaaagcagcacttataaatcagcctaacataagatctctctgatgtgtttgtgattctttcaaatccctatgtgccattatatttctttatttcctaaaacaggcaaaataagctcaagtttatgtactctgagtttttaaaacactggagtgatgttgctgaccagccgtttcctgtacctctctaagttgggtatttgggacttaagggattaagtttttcacctagacttagttacacacaatcttggcatttcctagcctagaggtttgtagcagggtacaagccccactcctcccccttcctttgctcccctgagtttggttttggcttaccataacattgttttgaccattcctagcctaatacaatagcctaacataatgtaagattaactggctttacgatttctattctctgctctcagtgataagaaacaaatattagctaccctgctaccctggttgaagccttccaaggctggctatgccctaggcatgggctcatccttgggtgtatcttgccttgcaggaagaccagtggaccgattgtgattctcaaaagctctgtgttgtcacctgtgcccttgccccttgctcttatcttggtccgtgtatctgggagttcttccaccttatcttggccaattcctaccttcgttcattcctcatgaggttgggtaaaagctccctccggctcccatgatgctgtgcatatacctagcaaaaagcaattattggacacattggagtgcaatattattaatagcattaatactactaataatgtgggcaatagtgattgtttttaaaaggcagtatactcttaccagtgcgaggtagctggggcctgtgatagtttttagagataagttcttcaggcaactgtgtattttacactagtcaagtaatcctagatatccgtggtttttcttaagaaagttggctcgtaatatgatttaatattcaaagtagagtcatctacctattagcttgctggcgtggtcctagtttatgcctgtttcagcatgattgttgagtaccctgtttcatccttagcattttcttgattttgttgttaaatgatgtatacccttatttccattgaatctgtgcttccacccccccaactgaagttgtcttccctttgcttggccacccttacagcctcttggatggtgtatcctacagtgtaagcactaaactgaagaggcagtgacctgagcactttggattttgttcattgtaatcaattccatgacaaaatgattgcatgagaaggaattttaaattcataggatcagaatttaggtgaaaacaaccagcatatttgtttcttcaccctctcacctagaattagctttgacctacaggtcacagtgcaatccccttgtatttctaaggtgttttttatagttcatttgcagacaatgggttatgtgataacttttatcagtgatagattaaacagaataatgaccaagctttcaaccttaaggagtcaggccagtatttacaaaaggaggtctccatgaactccttaaatatgagttcccctaatatcatcttgccaggtactaaataacaactgatagcacaagctatagggaatttgaaagaattccatggatgggtgttgtctagggccttttgttgtttttgagacggggtctgactctcacccaggctggagtatagtgtggcgcaatcttggctcactgcaacttctgcctcccagattcaagcgattctcctgcctcagcctcccaagtagctgagactacaggtgtgcaccaccatgctcagctaatttttgtatttttagtacagatggggtttcaccatgttggccaggctggtcttgaactcctgatctcccaaagtgaggtcttgaactggtcttgaactcctccacctcccaaagtgctgggattacaggcgtgagccactgcacccggcctagggccatgtaaaaagccagatctgtgctgctgtctgtgtagaagggtagacaagtggatgagaagttcctgaactattcttggcccttttaccactaagtgaaagtaacttgctgccccaaagaaagatgtctcatcattcgacaggactttctagttgaacttcatgaaagcaagagatcctgtttttcttgctcaccactgtatcttgagacctgttgtagtgcctgcaatacttatttaataagttatttttaagtatcagttttgtgagctttaactctatgaggtctttgttgtttgactgtattttaactctggccatgacagcaagacaaagttccatttttattgagcttaaaaagaatcaaggccaggtgaagtggcttacgcctgtgatcccaacactttgtgaggctgcagcaggaggatctcttgagcccaggagtttgagaccgttctaggcaatgtagtgaggtccagactccacaaaataatttttttttaaattgcacgcctgtagtctcagctatcaggaggctgagatgggaggatgacttgagcccaggaaattgaagctgcagtgaattgtgattgcaccactgcactccagcctgggtgacagatcaagaccttgcctaaacaaaacaaaacaaacaaaaccccaaaaaacaaattgaaaatgttgattctttttactacaaacattatggcagcactaaaaacttcgtgggagtgtactgtggaaaatagtgtacttaattaattctcattgtaatcaggctaccaagagccttgtgttgctttaagagttataactgccaggcacagtggctcatgcctataatcccagcaccttgagaggccgaggcaggtggatcacctgagatcgggagtttgagaccagccgggccaatatggtgaaacaagctgtgtctctactaaatacaaaaaattagccgggcgtggtggcacatgcctgtaatcccagctgcttgggagactgagacaggagaattgcttgaacctggaaggcggaggttgcagtgagctgagattgcaacattgtactccagcctgggcaacaagagggaaactccatctcaaaaaaaaaaaaaaagttgtaactgaggctgggcatggtggctcatacctgtaatcccagcactttgaaaagccgaggcaggtagatcacttgagctcagaagttcgagactagcctgggcaacatgacaaaaccccatctctacaaaaaatacgaaaaattagctgggcgtggtggcatgcacctgtagtcctagctacctgggaggctgaggtgggaagattacttgaagctgcagtgagccatggttgtgccactgccctccagtctgggcaacaaagtgagaccctgtctcaaaaaaacaaaaaaaaattataactgatgtaaactggcagtttaggctgggtgtggtggctcaagcctgtaatcctagcactttgggaggccaaggcaggtggatcacctgagttcaggagttcgagaccagcgtggccaacatggtgaaaccttgtctctattaaaaataccaaaattagcaagatgtggtggtgggtgcctataattccagctactcaggaggctgaggcaggaggatcgctggagccagggaggcagaggttacagtaagcaaagatcactccacttcactccagcctgggcaaaagagtgagacatatcaaaaaataaacaaataaataaataaataagtggcagttcatcatttaactccaaagactttgcgtacatttctactgaaaacaatctgagctgattagaaccctgccattttatagcctttagctcgatctccgaccgttcatttaaaaaaattctacttcaggccgggcatggtggctcaagcctgtaatcccatcactgtaggaggccaaagtgggcagatcacttaaggtcaggagtttgagaccagcctggccaccatggtgaaaccccatctctactaaaaatacaaaaattagccgggcttggtggtgagcacctgtaatcccaccctgccgagtggcaggctgaggcaggagaatcgcttgagcccaagagccggaggttgcagtgagccaagcttgcaccattgcactccagcctaggcaacagagtgtgactccatctcaagaaaaaaaaaattctatttcattttacaatatgcagatatatgtccatacacatgcataatataaatgtataccatatttgtgagaatatgcatatatgtacacattagatacacaatacaagcacaatacatatgtcttttgcccaagatacagcattttgtaaaggagacaggaatttagtaatatatgttccagaaacagtacacaagagaattcgccgagatgagaaagttgtcactaggaatggggagtggtaagatgtagaaggtataattgttcttaaagttctactgccaactctttccaattaattacccactctgccatgctttatggacaggaggttgtcggacactgtcaattaataaatatttgagcatgatacactgcttggagctcctctaatataggagagtgatatcctagtgcatgttacagagggagtgtccacacagttcctattgtcatttgatgagttacttttcaggggccttgtacctgagcaagttgtcctctttttgatggatttcagattgagttacctgcattgtcttgagattgcagcgtgtttcctccactgtacggcgtagtcagcagatctattagttaaactccagtgggccctcagtcactaaatctatcctctgtgttgaaggctttctgcatttgcctttcaataaaggtttagaataactccttaaaaaa

SEQ ID NO: 37 - protein, 161 aa, Homo sapiens
  MISC_FEATURE: Thy1 Accession number NM_006288
MNLAISIALLLTVLQVSRGQKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKCEGISLLAQNTSWLLLLLLSLSLLQATDFMSL

SEQ ID NO: 38 - DNA, 3008 bp, Homo sapiens
  misc_feature: cDNA Thy1
actaggcagggatgagcaagaggaatggctcacccttgagagctggggtccatagcccaggtcagttctccagctctcccacttaccagccaagacaggaggtgaggattgagatgggatgaacccagcaggcggccatgggttaaaggtcgccatgaatgtaatgtgcccagcacagtgcctgctaaaaggcaacactcccttcctggtctgaagaccaaacaagcagactgtactcaggaaagccagaagaaccttccagctgtctggaccagaaggtgccagcccaggggctgaagaagacgtaatgcccagagcaaaaagcgcctgcagccccctgaagggctgggtgctctggaatagatgagggggcgaaatggggctggggaccagggacggacagggtgggtccagcacctgcctcgcttccgaagggctgctccaacactgaaaaacacccaaccagcttcctttcagaaagactggaatattccaaaacttctcactggaggctccggaggaggtgggctccagctgaaaaggaaatgtggaggcgtgggcgctcccggcctgcatcctgcacctcttacactttggttttcccacagactcctgaagaataggtcagaagaaagggttaaagccttaaaaggggaacaaccattgcggggctcagggaggaggataatgttctttgggctgccgcaccctgatccccggggtcccgaaccctcccgtccctggccaggcctgccagccacagggtgagggcccccttccgccgcaacctgccactctcacaccaatgcgggaccgccttctcttccttccccaccccccaccccaccctgccgtcctttctcccccaatctccgcctctgattggctgagcccccggctccccgctccccctctcctccatccccggtgaaaactgcgggctccgagctgggtgcagcaaccggaggcggcggcgcgtctggaggaggctgcagcagcggaagaccccagtccagatccaggactgagatcccagaaccatgaacctggccatcagcatcgctctcctgctaacagtcttgcaggtctcccgagggcagaaggtgaccagcctaacggcctgcctagtggaccagagccttcgtctggactgccgccatgagaataccagcagttcacccatccagtacgagttcagcctgacccgtgagacaaagaagcacgtgctctttggcactgtgggggtgcctgagcacacataccgctcccgaaccaacttcaccagcaaatacaacatgaaggtcctctacttatccgccttcactagcaaggacgagggcacctacacgtgtgcactccaccactctggccattccccacccatctcctcccagaacgtcacagtgctcagagacaaactggtcaagtgtgagggcatcagcctgctggctcagaacacctcgtggctgctgctgctcctgctctccctctccctcctccaggccacggatttcatgtccctgtgactggtggggcccatggaggagacaggaagcctcaagttccagtgcagagatcctacttctctgagtcagctgaccccctccccccaatccctcaaaccttgaggagaagtggggaccccacccctcatcaggagttccagtgctgcatgcgattatctacccacgtccacgcggccacctcaccctctccgcacacctctggctgtctttttgtactttttgttccagagctgcttctgtctggtttatttaggttttatccttccttttctttgagagttcgtgaagagggaagccaggattggggacctgatggagagtgagagcatgtgaggggtagtgggatggtggggtaccagccactggaggggtcatccttgcccatcgggaccagaaacctgggagagacttggatgaggagtggttgggctgtgcctgggcctagcacggacatggtctgtcctgacagcactcctcggcaggcatggctggtgcctgaagaccccagatgtgagggcaccaccaagaatttgtggcctaccttgtgagggagagaactgagcatctccagcattctcagccacaaccaaaaaaaaataaaaagggcagccctccttaccactgtggaagtccctcagaggccttggggcatgacccagtgaagatgcaggtttgaccaggaaagcagcgctagtggagggttggagaaggaggtaaaggatgagggttcatcatccctccctgcctaaggaagctaaaagcatggccctgctgcccctccctgcctccacccacagtggagagggctacaaaggaggacaagaccctctcaggctgtcccaagctcccaagagcttccagagctctgacccacagcctccaagtcaggtggggtggagtcccagagctgcacagggtttggcccaagtttctaagggaggcacttcctcccctcgcccatcagtgccagcccctgctggctggtgcctgagcccctcagacagccccctgccccgcaggcctgccttctcagggacttctgcggggcctgaggcaagccatggagtgagacccaggagccggacacttctcaggaaatggcttttcccaacccccagcccccacccggtggttcttcctgttctgtgactgtgtatagtgccaccacagcttatggcatctcattgaggacaaagaaaactgcacaataaaaccaagcctctggaatctgtcctcgtgtccacctggccttcgctcctccagcagtgcctgcctgcccccgcttcgctggggtctccacgggtgaggctggggaacgccacctcttcctcttccctgacttctccccaaccacttagtagcaacgctaccccaggggctaatgactgcacactgggcttcttttcagaatgaccctaacgagacacatttgcccaaataaacgaacatcccatgtctgctgactcaaaaaaaaaaaaaaaa

SEQ ID NO: 39 - protein, 335 aa, Homo sapiens
  MISC_FEATURE: GLRX3 Accession number NM_001199868
MAAGAAEAAVAAVEEVGSAGQFEELLRLKAKSLLVVHFWAPWAPQCAQMNEVMAELAKELPQVSFVKLEAEGVPEVSEKYEISSVPTFLFFKNSQKIDRLDGAHAPELTKKVQRHASSGSFLPSANEHLKEDLNLRLKKLTHAAPCMLFMKGTPQEPRCGFSKQMVEILHKHNIQFSSFDIFSDEEVRQGLKAYSSWPTYPQLYVSGELIGGLDIIKELEASEELDTICPKAPKLEERLKVLTNKASVMLFMKGNKQEAKCGFSKQILEILNSTGVEYETFDILEDEEVRQGLKAYSNWPTYPQLYVKGELVGGLDIVKELKENGELLPILRGEN

SEQ ID NO: 40 - DNA, 1365 bp, Homo sapiens
  misc_feature: cDNA GLRX3
acatccggccgccggcactggattgcttctgtctggcggcggcagcatggcggcgggggcggctgaggcagctgtagcggccgtggaggaggtcggctcagccgggcagtttgaggagctgctgcgcctcaaagccaagtccctccttgtggtccatttctgggcaccatgggctccacagtgtgcacagatgaacgaagttatggcagagttagctaaagaactccctcaagtttcatttgtgaagttggaagctgaaggtgttcctgaagtatctgaaaaatatgaaattagctctgttcccacttttctgtttttcaagaattctcagaaaatcgaccgattagatggtgcacatgccccagagttgaccaaaaaagttcagcgacatgcatctagtggctccttcctacccagcgctaatgaacatcttaaagaagatctcaaccttcgcttgaagaaattgactcatgctgccccctgcatgctgtttatgaaaggaactcctcaagaaccacgctgtggtttcagcaagcagatggtggaaattcttcacaaacataatattcagtttagcagttttgatatcttctcagatgaagaggttcgacagggactcaaagcctattccagttggcctacctatcctcagctctatgtttctggagagctcataggaggacttgatataattaaggagctagaagcatctgaagaactagatacaatttgtcccaaagctcccaaattagaggaaaggctcaaagtgctgacaaataaagcttctgtgatgctctttatgaaaggaaacaaacaggaagcaaaatgtggattcagcaaacaaattctggaaatactaaatagtactggtgttgaatatgaaacattcgatatattggaggatgaagaagttcggcaaggattaaaagcttactcaaattggccaacataccctcagctgtatgtgaaaggggagctggtgggaggattggatattgtgaaggaactgaaagaaaatggtgaattgctgcctatactgagaggagaaaattaataaatcttaaacttggtgcccaactattacggggtctggctctgtcacccaggctggagtgcagtggcacgattatggctcattgcagcctcgacttctcggggccaagcgatcctcctgcctcagccttctgagtagctgggaccacaggcgtgcaccaccatgcccacctaattttttatttcttgtagagatgaggtctcctgcctcagcctcccaaagtgctggaatttacaggtaggaccaccacacttggtccacttacttataataaacattgatttggtcgttcaggttcaaaaaaaaaaaaa

SEQ ID NO: 41 - protein, 2409 aa, Homo sapiens
  MISC_FEATURE: VCAN NM_001164097
MFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKRRMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVSVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPGPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 42 - DNA, 9455 bp, Homo sapiens
  misc_feature: cDNA VCAN
cttcttctcgctgagtctcctcctcggctctgacggtacagtgatataatgatgatgggtgtcacaacccgcatttgaacttgcaggcgagctgccccgagcctttctggggaagaactccaggcgtgcggacgcaacagccgagaacattaggtgttgtggacaggagctgggaccaagatcttcggccagccccgcatcctcccgcatcttccagcaccgtcccgcaccctccgcatccttccccgggccaccacgcttcctatgtgacccgcctgggcaacgccgaacccagtcgcgcagcgctgcagtgaattttccccccaaactgcaataagccgccttccaaggccaagatgttcataaatataaagagcatcttatggatgtgttcaaccttaatagtaacccatgcgctacataaagtcaaagtgggaaaaagcccaccggtgaggggctccctctctggaaaagtcagcctaccttgtcatttttcaacgatgcctactttgccacccagttacaacaccagtgaatttctccgcatcaaatggtctaagattgaagtggacaaaaatggaaaagatttgaaagagactactgtccttgtggcccaaaatggaaatatcaagattggtcaggactacaaagggagagtgtctgtgcccacacatcccgaggctgtgggcgatgcctccctcactgtggtcaagctgctggcaagtgatgcgggtctttaccgctgtgacgtcatgtacgggattgaagacacacaagacacggtgtcactgactgtggatggggttgtgtttcactacagggcggcaaccagcaggtacacactgaattttgaggctgctcagaaggcttgtttggacgttggggcagtcatagcaactccagagcagctctttgctgcctatgaagatggatttgagcagtgtgacgcaggctggctggctgatcagactgtcagatatcccatccgggctcccagagtaggctgttatggagataagatgggaaaggcaggagtcaggacttatggattccgttctccccaggaaacttacgatgtgtattgttatgtggatcatctggatggtgatgtgttccacctcactgtccccagtaaattcaccttcgaggaggctgcaaaagagtgtgaaaaccaggatgccaggctggcaacagtgggggaactccaggcggcatggaggaacggctttgaccagtgcgattacgggtggctgtcggatgccagcgtgcgccaccctgtgactgtggccagggcccagtgtggaggtggtctacttggggtgagaaccctgtatcgttttgagaaccagacaggcttccctccccctgatagcagatttgatgcctactgctttaaacgtcgaatgagtgatttgagtgtaattggtcatccaatagattcagaatctaaagaagatgaaccttgtagtgaagaaacagatccagtgcatgatctaatggctgaaattttacctgaattccctgacataattgaaatagacctataccacagtgaagaaaatgaagaagaagaagaagagtgtgcaaatgctactgatgtgacaaccaccccatctgtgcagtacataaatgggaagcatctcgttaccactgtgcccaaggacccagaagctgcagaagctaggcgtggccagtttgaaagtgttgcaccttctcagaatttctcggacagctctgaaagtgatactcatccatttgtaatagccaaaacggaattgtctactgctgtgcaacctaatgaatctacagaaacaactgagtctcttgaagttacatggaagcctgagacttaccctgaaacatcagaacatttttcaggtggtgagcctgatgttttccccacagtcccattccatgaggaatttgaaagtggaacagccaaaaaaggggcagaatcagtcacagagagagatactgaagttggtcatcaggcacatgaacatactgaacctgtatctctgtttcctgaagagtcttcaggagagattgccattgaccaagaatctcagaaaatagcctttgcaagggctacagaagtaacatttggtgaagaggtagaaaaaagtacttctgtcacatacactcccactatagttccaagttctgcatcagcatatgtttcagaggaagaagcagttaccctaataggaaatccttggccagatgacctgttgtctaccaaagaaagctgggtagaagcaactcctagacaagttgtagagctctcagggagttcttcgattccaattacagaaggctctggagaagcagaagaagatgaagatacaatgttcaccatggtaactgatttatcacagagaaatactactgatacactcattactttagacactagcaggataatcacagaaagcttttttgaggttcctgcaaccaccatttatccagtttctgaacaaccttctgcaaaagtggtgcctaccaagtttgtaagtgaaacagacacttctgagtggatttccagtaccactgttgaggaaaagaaaaggaaggaggaggagggaactacaggtacggcttctacatttgaggtatattcatctacacagagatcggatcaattaattttaccctttgaattagaaagtccaaatgtagctacatctagtgattcaggtaccaggaaaagttttatgtccttgacaacaccaacacagtctgaaagggaaatgacagattctactcctgtctttacagaaacaaatacattagaaaatttgggggcacagaccactgagcacagcagtatccatcaacctggggttcaggaagggctgaccactctcccacgtagtcctgcctctgtctttatggagcagggctctggagaagctgctgccgacccagaaaccaccactgtttcttcattttcattaaacgtagagtatgcaattcaagccgaaaaggaagtagctggcactttgtctccgcatgtggaaactacattctccactgagccaacaggactggttttgagtacagtaatggacagagtagttgctgaaaatataacccaaacatccagggaaatagtgatttcagagcgattaggagaaccaaattatggggcagaaataaggggcttttccacaggttttcctttggaggaagatttcagtggtgactttagagaatactcaacagtgtctcatcccatagcaaaagaagaaacggtaatgatggaaggctctggagatgcagcatttagggacacccagacttcaccatctacagtacctacttcagttcacatcagtcacatatctgactcagaaggacccagtagcaccatggtcagcacttcagccttcccctgggaagagtttacatcctcagctgagggctcaggtgagcaactggtcacagtcagcagctctgttgttccagtgcttcccagtgctgtgcaaaagttttctggtacagcttcctccattatcgacgaaggattgggagaagtgggtactgtcaatgaaattgatagaagatccaccattttaccaacagcagaagtggaaggtacgaaagctccagtagagaaggaggaagtaaaggtcagtggcacagtttcaacaaactttccccaaactatagagccagccaaattatggtctaggcaagaagtcaaccctgtaagacaagaaattgaaagtgaaacaacatcagaggaacaaattcaagaagaaaagtcatttgaatcccctcaaaactctcctgcaacagaacaaacaatctttgattcacagacatttactgaaactgaactcaaaaccacagattattctgtactaacaacaaagaaaacttacagtgatgataaagaaatgaaggaggaagacacttctttagttaacatgtctactccagatccagatgcaaatggcttggaatcttacacaactctccctgaagctactgaaaagtcacattttttcttagctactgcattagtaactgaatctataccagctgaacatgtagtcacagattcaccaatcaaaaaggaagaaagtacaaaacattttccgaaaggcatgagaccaacaattcaagagtcagatactgagctcttattctctggactgggatcaggagaagaagttttacctactctaccaacagagtcagtgaattttactgaagtggaacaaatcaataacacattatatccccacacttctcaagtggaaagtacctcaagtgacaaaattgaagactttaacagaatggaaaatgtggcaaaagaagttggaccactcgtatctcaaacagacatctttgaaggtagtgggtcagtaaccagcacaacattaatagaaattttaagtgacactggagcagaaggacccacggtggcacctctccctttctccacggacatcggacatcctcaaaatcagactgtcaggtgggcagaagaaatccagactagtagaccacaaaccataactgaacaagactctaacaagaattcttcaacagcagaaattaacgaaacaacaacctcatctactgattttctggctagagcttatggttttgaaatggccaaagaatttgttacatcagcaccaaaaccatctgacttgtattatgaaccttctggagaaggatctggagaagtggatattgttgattcatttcacacttctgcaactactcaggcaaccagacaagaaagcagcaccacatttgtttctgatgggtccctggaaaaacatcctgaggtgccaagcgctaaagctgttactgctgatggattcccaacagtttcagtgatgctgcctcttcattcagagcagaacaaaagctcccctgatccaactagcacactgtcaaatacagtgtcatatgagaggtccacagacggtagtttccaagaccgtttcagggaattcgaggattccaccttaaaacctaacagaaaaaaacccactgaaaatattatcatagacctggacaaagaggacaaggatttaatattgacaattacagagagtaccatccttgaaattctacctgagctgacatcggataaaaatactatcatagatattgatcatactaaacctgtgtatgaagacattcttggaatgcaaacagatatagatacagaggtaccatcagaaccacatgacagtaatgatgaaagtaatgatgacagcactcaagttcaagagatctatgaggcagctgtcaacctttctttaactgaggaaacatttgagggctctgctgatgttctggctagctacactcaggcaacacatgatgaatcaatgacttatgaagatagaagccaactagatcacatgggctttcacttcacaactgggatccctgctcctagcacagaaacagaattagacgttttacttcccacggcaacatccctgccaattcctcgtaagtctgccacagttattccagagattgaaggaataaaagctgaagcaaaagccctggatgacatgtttgaatcaagcactttgtctgatggtcaagctattgcagaccaaagtgaaataataccaacattgggccaatttgaaaggactcaggaggagtatgaagacaaaaaacatgctggtccttcttttcagccagaattctcttcaggagctgaggaggcattagtagaccatactccctatctaagtattgctactacccaccttatggatcagagtgtaacagaggtgcctgatgtgatggaaggatccaatcccccatattacactgatacaacattagcagtttcaacatttgcgaagttgtcttctcagacaccatcatctcccctcactatctactcaggcagtgaagcctctggacacacagagatcccccagcccagtgctctgccaggaatagacgtcggctcatctgtaatgtccccacaggattcttttaaggaaattcatgtaaatattgaagcgactttcaaaccatcaagtgaggaataccttcacataactgagcctccctctttatctcctgacacaaaattagaaccttcagaagatgatggtaaacctgagttattagaagaaatggaagcttctcccacagaacttattgctgtggaaggaactgagattctccaagatttccaaaacaaaaccgatggtcaagtttctggagaagcaatcaagatgtttcccaccattaaaacacctgaggctggaactgttattacaactgccgatgaaattgaattagaaggtgctacacagtggccacactctacttctgcttctgccacctatggggtcgaggcaggtgtggtgccttggctaagtccacagacttctgagaggcccacgctttcttcttctccagaaataaaccctgaaactcaagcagctttaatcagagggcaggattccacgatagcagcatcagaacagcaagtggcagcgagaattcttgattccaatgatcaggcaacagtaaaccctgtggaatttaatactgaggttgcaacaccaccattttcccttctggagacttctaatgaaacagatttcctgattggcattaatgaagagtcagtggaaggcacggcaatctatttaccaggacctgatcgctgcaaaatgaacccgtgccttaacggaggcacctgttatcctactgaaacttcctacgtatgcacctgtgtgccaggatacagcggagaccagtgtgaacttgattttgatgaatgtcactctaatccctgtcgtaatggagccacttgtgttgatggttttaacacattcaggtgcctctgccttccaagttatgttggtgcactttgtgagcaagataccgagacatgtgactatggctggcacaaattccaagggcagtgctacaaatactttgcccatcgacgcacatgggatgcagctgaacgggaatgccgtctgcagggtgcccatctcacaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagtcgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtggcaggagtcgaggcgctgatccctaaaatggcgaacatgtgttttcatcatttcagccaaagtcctaacttcctgtgcctttcctatcacctcgagaagtaattatcagttggtttggatttttggaccaccgttcagtcattttgggttgccgtgctcccaaaacattttaaatgaaagtattggcattcaaaaagacagcagacaaaatgaaagaaaatgagagcagaaagtaagcatttccagcctatctaatttctttagttttctatttgcctccagtgcagtccatttcctaatgtataccagcctactgtactatttaaaatgctcaatttcagcaccgatggccatgtaaataagatgatttaatgttgattttaatcctgtatataaaataaaaagtcacaatgagtttgggcatatttaatgatgattatggagccttagaggtctttaatcattggttcggctgcttttatgtagtttaggctggaaatggtttcacttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgcattcctttttcttcacttacaagaaaggcctgaatggaggacttttctgtaaccaggaacattttttaggggtcaaagtgctaataattaactcaaccaggtctactttttaatggctttcataacactaactcataaggttaccgatcaatgcatttcatacggatatagacctagggctctggagggtgggggattgttaaaacacatgcaaaaaaaaaaaaaaaaaaaaaaaaagaaattttgtatatataaccattttaatcttttataaagttttgaatgttcatgtatgaatgctgcagctgtgaagcatacataaataaatgaagtaagccatactgatttaatttattggatgttattttccctaagacctgaaaatgaacatagtatgctagttatttttcagtgttagccttttactttcctcacacaatttggaatcatataatataggtactttgtccctgattaaataatgtgacggatagaatgcatcaagtgtttattatgaaaagagtggaaaagtatatagcttttagcaaaaggtgtttgcccattctaagaaatgagcgaatatatagaaatagtgtgggcatttcttcctgttaggtggagtgtatgtgttgacatttctccccatctcttcccactctgttttctccccattatttgaataaagtgactgctgaagatgactttgaatccttatccacttaatttaatgtttaaagaaaaacctgtaatggaaagtaagactccttccctaatttcagtttagagcaacttgaagaagagtagacaaaaaataaaatgcacatagaaaaagagaaaaagggcacaaagggattggcccaatattgattctttttttataaaacctcctttggcttagaaggaatgactctagctacaataatacacagtatgtttaagcaggttcccttggttgttgcattaaatgtaatccacctttaggtattttagagcacagaacaacactgtgttgatctagtaggtttctatttttcctttctctttacaatgcacataatactttcctgtatttatatcataacgtgtatagtgtaaaatgtgaatgactttttttgtgaatgaaaatctaaaatctttgtaactttttatatctgcttttgtttcaccaaagaaacctaaaatccttcttttactacac

SEQ ID NO: 43 - protein, 443 aa, Homo sapiens
  MISC_FEATURE: CPM ACCESSION   NM_001874
MDFPCLWLGLLLPLVAALDFNYHRQEGMEAFLKTVAQNYSSVTHLHSIGKSVKGRNLWVLVVGRFPKEHRIGIPEFKYVANMHGDETVGRELLLHLIDYLVTSDGKDPEITNLINSTRIHIMPSMNPDGFEAVKKPDCYYSIGRENYNQYDLNRNFPDAFEYNNVSRQPETVAVMKWLKTETFVLSANLHGGALVASYPFDNGVQATGALYSRSLTPDDDVFQYLAHTYASRNPNMKKGDECKNKMNFPNGVTNGYSWYPLQGGMQDYNYIWAQCFEITLELSCCKYPREEKLPSFWNNNKASLIEYIKQVHLGVKGQVFDQNGNPLPNVIVEVQDRKHICPYRTNKYGEYYLLLLPGSYIINVTVPGHDPHITKVIIPEKSQNFSALKKDILLPFQGQLDSIPVSNPSCPMIPLYRNLPDHSAATKPSLFLFLVSLLHIFFK

SEQ ID NO: 44 - DNA, 6683 bp, Homo sapiens
  misc_feature: cDNA CPM
gcatttcttccttctgcgtatgggacaggaccctttctggaatgggggtcttatgacctacaatcaaacaagaacatggacttcccgtgcctctggctagggctgttgctgcctttggtagctgcgctggatttcaactaccaccgccaggaagggatggaagcgtttttgaagactgttgcccaaaactacagttctgtcactcacttacacagtattgggaaatctgtgaaaggtagaaacctgtgggttcttgttgtggggcggtttccaaaggaacacagaattgggattccagagttcaaatacgtggcaaatatgcatggagatgagactgttgggcgggagctgctgctccatctgattgactatctcgtaaccagtgatggcaaagaccctgaaatcacaaatctgatcaatagtacccggatacacatcatgccttccatgaacccagatggatttgaagccgtcaaaaagcctgactgttattacagcatcggaagggaaaattataaccagtatgacttgaatcgaaatttccccgatgcttttgaatataataatgtctcaaggcagcctgaaactgtggcagtcatgaagtggctgaaaacagagacgtttgtcctctctgcaaacctccatggtggtgccctcgtggccagttacccatttgataatggtgttcaagcaactggggcattatactcccgaagcttaacgcctgatgatgatgtttttcaatatcttgcacatacctatgcttcaagaaatcccaacatgaagaaaggagacgagtgtaaaaacaaaatgaactttcctaatggtgttacaaatggatactcttggtatccactccaaggtggaatgcaagattacaactacatctgggcccagtgttttgaaattacgttggagctgtcatgctgtaaatatcctcgtgaggagaagcttccatccttttggaataataacaaagcctcattaattgaatatataaagcaggtgcacctaggtgtaaagggtcaagtttttgatcagaatggaaatccattacccaatgtaattgtggaagtccaagacagaaaacatatctgcccctatagaaccaacaaatatggagagtattatctccttctcttgcctgggtcttatataataaatgttacagtccctggacatgatccacacatcacaaaggtgattattccggagaaatcccagaacttcagtgctcttaaaaaggatattctacttccattccaagggcaattggattctatcccagtatcaaatccttcatgcccaatgattcctctatacagaaatttgccagaccactcagctgcaacaaagcctagtttgttcttatttttagtgagtcttttgcacatattcttcaaataaagtaaaatgtgaaactcaacccacatcaccacctggaatcagggattgctcactccaggttactgcaaccctaactcactctagtgggaccttgactggagaaactccacgatcttcctgaagaagagaaatggatgtttccaaattccacaataagcaatatgtggtgataatgaaaagaatgattcagtcttgacggtgaatggaagacacttacctaacaagtactgctcatttacactcaaattaatcttgaagtagtcttaaaatgtgtaagaagttaaaacttgagaagcaaaaaaatgcctgcaaaaagaagatcattttgtatacagagaaccggatgaatataagcaatgaagatgaacatttattgatcttctacatacaagacttcaccataaggccaggagcagtggctcacaccttgtaatcccagcactttgggaggccaaggtgggcggatcaccctgaggtcaggagttcaaaaccagcctgaccaacatggtgaaaccctgtctctactaaatattagcggggtgtggtggcgggcacctgtaatcgcagcctttcaggaggctgagacaggagaatcgcttgaaccctagaggcggagtttgcagtgagccgagatagtgccattgtactccagcttgggcaacagagtaagactctgtctcaaaaaaaaaaaaacaaaaacaaacaaacaaaaaaaacacctcaccatgagtgctacatgtgaatagatattaagtgccatatataattagttctcagaagaagggagaaatgatcataggactgggaattgttttgcaaacgttctaggagatgtgagagaaaatatgtaaccacatcttagtggcccaagaaaatacaggcctgaagggataagattgtgtctctatagagcttcaaagcatacaggtcaattaagaaagcccctctctctccagagccgtttccctagcttttggcacctggatgccacagtcctccattaggctgatgactccaaagatgtaactctagcctcttgcctgagcttcagactcgcgtcccactgcccacaggacacatccacctggatgtgactcacaggtacctccaacccatcatgtggagatactcatcctgttccccctagagctgctcttcctgctgcattctctctctcaattactgggaccaccaagctaggaacctgggagtcatccttgatactttctcttcctccttaatcctgtgtattcagcaagtaactaaaggttggtgttggccaggcatggtggctcatgcctgtaatcccagcattttgggaggccaaggcgggcggatcacttgaggtcaggagctcaagaccagcctggccaacatggtgaaaccccatctctactaaaaaaaaaaaaaaaattagtcgggcgtggtggtgcatgcctgtaatcccagctactggggaggctgaggcaggagaatcgcttgaacctgggaggcagaggttgcagtgagccgggattgcgccattgtactccagcctgggtgaagaagtgagactctgtcttaaaaaaaaaaattggtgctgataaatattgatgaattctgctctctgctctctatggttgtcaacactgcagagttgaggcctcatatctcacctgcactgctgcaacagcttactggtcccttgctcccagccttctcctcttcagtccatcgtccacacagcactggggaaggggagccacttgaaacaaaagtcaacaactggttgtagttcataaacacagagctgtttgtgtcccctgtatctggaatgccattatgacccactacattttttctttcctacccctcttaaaactcagttcaggtagcagctccactaggaagccttggctgaccataatcccattcaattccatttcacctcttcgcaggcagtctggggttagggaccctttctctttgctccccaaaataaactggttatctctactattggatttacaacattgtattataatcttctccatgtgtgccttctctagtagaatgtgagctctttgaggccaaggtctatttaatttgtttgaaaaattcattgttatatcctcaaagcctagcacatagtaggtactgaatgaatgaatgaacaaggggtgccaggagactgctactcccagtccttcccagaaactgcctagggctttgagtcattttatgaagctaggtcttaatgcgtaggcaacctcccagctcactatgaacgctgacagaagagtgttttcatgtctataatcaagaattccagatacattccttttactgaaccttgaattgatcctaagattggtagtaaaggtattatgttacctcctaacagcactacaaagtacctttttttatcagaaaaaaattttaccattaggactcaatttgaagtactaatgcttctcaagttctccactatgagagttaccctgtattagaccgttacctataagaattaaggggtaaagcactaaacagaaaagaaaaaaaaaatagcaactctggtgagcagatttctttcctttcttccttccttctcctcttcctaccttcctccctcctttccctctcctccccttctctccctttccctccccttcccttccttttcttctttcctccgctcccctccccttccctccccttcccatccttctttctcttttttttacttaatccccagtgtgacagtaatataggctgatttctagaagtgtggtgtattactcatggaaagtgagttgccttggttattactttcaattgaaagttctatgggatctagaaatgagacatactggcatggagagtgagaacgacaaaggaatgaagagctacaggagcatttaggccatttctatgccaagcttattctacatgcacaaaatcatacatgttaataaatataaacaaattggaggcttatttaaaccaattatgaaatctggtaatttgtgcagcagcaatagatgataaccaaaaaaaactcataataatctgaatatcttgatcatttgtatttaaagaagcagtaattatatacttgaaagtacataatatagtattgcaaaaatgactttggtatattacaaattaaaagtatataagatgaaacttgatttgctatcaagccccaagcaatttttcaactgggcattgaattctaacttttctaagatagcaatttttgaagagacacgaacaaaaatctgaattagttcatgagccttaatgtaaatctcttgctgaaatagtttttaaaatcagaatttagttatctatcagactcaaaatcatttaaagactaacaaaacacaatcatgatattctaactgtggtcaaaccaggtacccaagccacctccctgcccaacgcctttccggcttttcccctccctcttgggctggtggttatgctcctccagctctagttcagctataattccttttatagagaaaccaacctgatacacactttcatgatgggagaaaaatgtgggagtgaaatggtatttagaaagcagcagtcaggcacggtggctcatgcctgtaatcccagcactttgggaggctgaggcaggcggatcacttgaggtcaggagctcgagaccagcctggccaacacggtgaaaccccatctctactaaaaaaaaatacaaaaattagccgggcgtggtggcaggcacctgtaatcccagctacttgggaggctgaggcaggagaaatcgcctgaacccagaaggcagaggttgcagtgagccaagatcacatcactgcactgcactccagccggggtgacagagcgaacctctgtctcaaaaaaaaaaaaagaaaaaagaaagaaagaaaaaaggcagaagccctggattcaaatccgccacacattcagtttctttatctgtaaaatggagaccaccccccgccacgctgaacggtgattctgtgactggtaagagatgctacatttttggtgcttgttcaggtggaggaaagatgatagttaacactcaggtaataagtattttgaaggcagtataatataccttcttaaagagtatacctactcaaatgttggtaaatgttgacatgattgaatctaaatggcaaagagtattttagaaaaacattaagtccctgcagataaatgacagtgttgatttggatgcttaattacattcagacatgaactgttggatgtatctgaaatgttaaaagctttttctcaacatttccaaaagtctttccaagaaatcaatgttatgttttgttccagaagcaaatttgcatttgtgatctgtttctaaaaatggtacaagttagctctgtttagaaagtaaaaatatctgatgttagattggaagtatctcttcctggggaatccagaaagataagcatagcatattgtcttactgcaatagataagttgcttattgagaagtctggttgttattctatatggtaacaatacagttgatgtatattttatgatagatcctttatattttcctcatgactttagaagggggaagggggagaaaattatgatgaccagactagttaaagagcattgaaagtccacagtactgtagctaaagtagaagtttgggtttgttatagactttacattatatcaactaataagcagatactgtacagtattgctcaccattttatcatacttttgcatatgaactactccattgccttttatagatgttttatagctgatcttaccagttttcctggtaactttttttatttcttttttttttttttgagacggagtctcgccctaacacccaggttggagtgcagtgccgtgatctcggctcactgcaacctctgcctcccgggttcaagcaattctcctgtctcagcctcccgagtacctgggactaccggtgcctgtctccacgcccggctaattttttgtatttgtagtagagacggggtttcaccgtgttagccaggatggtctcgatctcctgacctcatgatctgcctgcctctgcctggacctcccaaagtgctgggattacaggcgtgagcccccgcgcccagccactttctttaatactataactaagaatttattaaaatgcacaaattgtctaagactgtaaagtttattggggagaggccatgactacctctgaatttagtaaatttaaaatatttctgattctcaataaagaactaatatccatataaaaaaaaaaaaaaaaa

SEQ ID NO: 45 - protein, 328 aa, Homo sapiens
  MISC_FEATURE: CD34 NM_001773
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 46 - DNA, 2816 bp, Homo sapiens
  misc_feature: CD34
ccttttttggcctcgacggcggcaacccagcctccctcctaacgccctccgcctttgggaccaaccaggggagctcaagttagtagcagccaaggagaggcgctgccttgccaagactaaaaagggaggggagaagagaggaaaaaagcaagaatcccccacccctctcccgggcggagggggcgggaagagcgcgtcctggccaagccgagtagtgtcttccactcggtgcgtctctctaggagccgcgcgggaaggatgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggagctggaaccctgaccactcttcaggaagaaaggagtctgcacatgcagctgcaccctccctccgatccttcctcccacctccccctcccccttctcccacccctgcccccacttcctgtttgggcccctctcccatccagtgtctcacagccctgcttaccagataatgctactttatttatacactgtctagggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtgactcggctaggtggggcaaggctgggcagtgtccgagagagcacccctctctgcatctgaccacgtgctacccccatgctggaggtgacatctcttacgcccaacccttccccactgcacacacctcagaggctgttcttggggccctacaccttgaggaggggcaggtaaactcctgtcctttacacattcggctccctggagccagactctggtcttctttgggtaaacgtgtgacgggggaaagccaaggtctggagaagctcccaggaacaatcgatggccttgcagcactcacacaggacccccttcccctaccccctcctctctgccgcaatacaggaacccccaggggaaagatgagcttttctaggctacaattttctcccaggaagctttgatttttaccgtttcttccctgtattttctttctctactttgaggaaaccaaagtaaccttttgcacctgctctcttgtaatgatatagccagaaaaacgtgttgccttgaaccacttccctcatctctcctccaagacactgtggacttggtcaccagctcctcccttgttctctaagttccactgagctccatgtgccccctctaccatttgcagagtcctgcacagttttctggctggagcctagaacaggcctcccaagttttaggacaaacagctcagttctagtctctctggggccacacagaaactctttttgggctcctttttctccctctggatcaaagtaggcaggaccatgggaccaggtcttggagctgagcctctcacctgtactcttccgaaaaatcctcttcctctgaggctggatcctagccttatcctctgatctccatggcttcctcctccctcctgccgactcctgggttgagctgttgcctcagtcccccaacagatgcttttctgtctctgcctccctcaccctgagccccttccttgctctgcacccccatatggtcatagcccagatcagctcctaacccttatcaccagctgcctcttctgtgggtgacccaggtccttgtttgctgttgatttctttccagaggggttgagcagggatcctggtttcaatgacggttggaaatagaaatttccagagaagagagtattgggtagatattttttctgaatacaaagtgatgtgtttaaatactgcaattaaagtgatactgaaacacaaaaaa

SEQ ID NO: 47 - protein, 1445 aa, Homo sapiens
  MISC_FEATURE: CD109 ACCESSION   NM_133493
MQGPPLLTAAHLLCVCTAALAVAPGPRFLVTAPGIIRPGGNVTIGVELLEHCPSQVTVKAELLKTASNLTVSVLEAEGVFEKGSFKTLTLPSLPLNSADEIYELRVTGRTQDEILFSNSTRLSFETKRISVFIQTDKALYKPKQEVKFRIVTLFSDFKPYKTSLNILIKDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGYRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPVKFLIDTHNRLLLQTAELAVVQPTAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 48 - DNA, 9170 bp, Homo sapiens
  misc_feature: cDNA CD109
gcgcgcccatttcagattactaaactcgaattaagagggaaaaaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatctatttaaaaaaaaaaaaaaaaaa

SEQ ID NO: 49 - protein, 313 aa, Homo sapiens
  MISC_FEATURE: ITLN1 Accession NM_017625
MNQLSFLLFLIATTRGWSTDEANTYFKEWTCSSSPSLPRSCKEIKDECPSAFDGLYFLRTENGVIYQTFCDMTSGGGGWTLVASVHENDMRGKCTVGDRWSSQQGSKAVYPEGDGNWANYNTFGSAEAATSDDYKNPGYYDIQAKDLGIWHVPNKSPMQHWRNSSLLRYRTDTGFLQTLGHNLFGIYQKYPVKYGEGKCWTDNGPVIPVVYDFGDAQKTASYYSPYGQREFTAGFVQFRVFNNERAANALCAGMRVTGCNTEHHCIGGGGYFPEASPQQCGDFSGFDWSGYGTHVGYSSSREITEAAVLLFYR

SEQ ID NO: 50 - DNA, 1209 bp, Homo sapiens
  misc_feature: cDNA ITLN1
aggagcgtttttggagaaagctgcactctgttgagctccagggcgcagtggagggagggagtgaaggagctctctgtacccaaggaaagtgcagctgagactcagacaagattacaatgaaccaactcagcttcctgctgtttctcatagcgaccaccagaggatggagtacagatgaggctaatacttacttcaaggaatggacctgttcttcgtctccatctctgcccagaagctgcaaggaaatcaaagacgaatgtcctagtgcatttgatggcctgtattttctccgcactgagaatggtgttatctaccagaccttctgtgacatgacctctgggggtggcggctggaccctggtggccagcgtgcacgagaatgacatgcgtgggaagtgcacggtgggcgatcgctggtccagtcagcagggcagcaaagcagtctacccagagggggacggcaactgggccaactacaacacctttggatctgcagaggcggccacgagcgatgactacaagaaccctggctactacgacatccaggccaaggacctgggcatctggcacgtgcccaataagtcccccatgcagcactggagaaacagctccctgctgaggtaccgcacggacactggcttcctccagacactgggacataatctgtttggcatctaccagaaatatccagtgaaatatggagaaggaaagtgttggactgacaacggcccggtgatccctgtggtctatgattttggcgacgcccagaaaacagcatcttattactcaccctatggccagcgggaattcactgcgggatttgttcagttcagggtatttaataacgagagagcagccaacgccttgtgtgctggaatgagggtcaccggatgtaacactgagcaccactgcattggtggaggaggatactttccagaggccagtccccagcagtgtggagatttttctggttttgattggagtggatatggaactcatgttggttacagcagcagccgtgagataactgaggcagctgtgcttctattctatcgttgagagttttgtgggagggaacccagacctctcctcccaaccatgagatcccaaggatggagaacaacttacccagtagctagaatgttaatggcagaagagaaaacaataaatcatattgactcaaaaaaaaaaaaaaaaaaaaaaaaaaaaa

SEQ ID NO: 51 - protein, 314 aa, Homo sapiens
  MISC_FEATURE: C1RL ACCESSION   NM_001297642
MPGPRVWGKYLWRSPHSKGCPGAMWWLLLWGVLQACPTRGSVLLAQELPQQLTSPGYPEPYGKGQESSTDIKAPEGFAVRLVFQDFDLEPSQDCAGDSVTISFVGSDPSQFCGQQGSPLGRPPGQREFVSSGRSLRLTFRTQPSSENKTAHLHKGFLALYQTVAVNYSQPISEASRGSEAINAPGDNPAKVQNHCQEPYYQAAAAASTPSLFLCLSSFTPQGHSPVQPQGPGKTDRMGRRFFSVCLSADGQSPPLPRIRRPSVLPEPSWATSPGKPSPVSTAVGAGPCWGTDGSSLLPTPSTPRTVFLSGRTRV

SEQ ID NO: 52 - DNA, 3450 bp, Homo sapiens
  misc_feature: cDNA C1RL
ttaacatttccaggaacttcctcctcccccaccggccttcaccttttgttccctatcctgggccagttctctcgcaggtcccagatgtccagttccagatgcctggacccagagtgtgggggaaatatctctggagaagccctcactccaaaggctgtccaggcgcaatgtggtggctgcttctctggggagtcctccaggcttgcccaacccggggctccgtcctcttggcccaagagctaccccagcagctgacatcccccgggtacccagagccgtatggcaaaggccaagagagcagcacggacatcaaggctccagagggctttgctgtgaggctcgtcttccaggacttcgacctggagccgtcccaggactgtgcaggggactctgtcacaatctcattcgtcggttcggatccaagccagttctgtggtcagcaaggctcccctctgggcaggccccctggtcagagggagtttgtatcctcagggaggagtttgcggctgaccttccgcacacagccttcctcggagaacaagactgcccacctccacaagggcttcctggccctctaccaaaccgtggctgtgaactatagtcagcccatcagcgaggccagcaggggctctgaggccatcaacgcacctggagacaaccctgccaaggtccagaaccactgccaggagccctattatcaggccgcggcagcagcttcaactccgagcctatttctttgcctctcctcatttacgccacaggggcactcacctgtgcaaccccagggacctggaaagacagacaggatggggaggaggttcttcagtgtatgcctgtctgcggacggccagtcacccccattgcccagaatcagacgaccctcggttcttccagagccaagctgggcaacttcccctggcaagccttcaccagtatccacggccgtgggggcggggccctgctgggggacagatggatcctcactgctgcccacaccatctaccccaaggacagtgtttctctcaggaagaaccagagtgtgaatgtgttcttgggccacacagccatagatgagatgctgaaactggggaaccaccctgtccaccgtgtcgttgtgcaccccgactaccgtcagaatgagtcccataactttagcggggacatcgccctcctggagctgcagcacagcatccccctgggccccaacgtcctcccggtctgtctgcccgataatgagaccctctaccgcagcggcttgttgggctacgtcagtgggtttggcatggagatgggctggctaactactgagctgaagtactcgaggctgcctgtagctcccagggaggcctgcaacgcctggctccaaaagagacagagacccgaggtgttttctgacaatatgttctgtgttggggatgagacgcaaaggcacagtgtctgccagggggacagtggcagcgtctatgtggtatgggacaatcatgcccatcactgggtggccacgggcattgtgtcctggggcatagggtgtggcgaagggtatgacttctacaccaaggtgctcagctatgtggactggatcaagggagtgatgaatggcaagaattgaccctgggggcttgaacagggactgaccagcacagtggaggccccaggcaacagagggcctggagtgaggactgaacactggggtaggggttgggggtggggggttgggggaggcagggaaatcctattcacatcactgttgcaccaagccactgcaagagaaacccccacccggcaagcccgccccatcccagacaggaagcagagtcccacagaccgctcctcctcaccctctacctccctgtgctcatgcactaggccccgggaagcctgtacatctcaacaactttcgccttgaatgtccttagaaccgccttcccctacttcatctgttgacacagcttttatactcacctgtggaagagtcagctactcacccgctattagagtatggaggaaggggttttcattgcattgcatttctgaaacattcctaagaccctttagttgaccttcaaatattcaagctattctgcagctccaagatgcaattatagaaacagctccttttttattttatgtcctctatatgccaggtgcttcacctgttatttcacttaatcctcataccatatttgcaaaggatgtgttattatctatgtgtgacaaatgaggaaactgaggctcaggggataaagggacttgcccaagtcccacagctggtgtgtgactgcagagactgtgctcttcccagtgtgctgcaatacttctcaaccctcctctaacctgctgtgtcacccgctttccctcccagcccccacatccttaccattttccctccctgggaattcctgcttctgcgaaaatggtatcctctagctcacactttcctaatggccccatctcctgcagaagccaggtgagcccagcactggactgaagttcttgcagacaccccacctgtgcccctatcatcaggggaactgctccacctgagaggaccaactctttaatttttagtaaaacctggaggtgatgggccgggcgcagtggctcacgcctgtaatcccaacaccttaggagtccgaggtgggtggatcacgaggtcaggagatccagcccatcctggccaacatggtgaaaccccatctctactaaaaatacaaaaattagccgggcgtggtgacacgtgcctgtagtcccagctactcgggaggctgaggcaggagaatcacttgaacctgggaggcggaggttgcagtgagctaagatcacgccactgcactccagcctgcggacagaccaagacttcatcccccccaaaaaaaaaagattggaggtgatttacagtgaaagacacaaataaaatacaactgttcaatggaaatagaaaataaacaccataaaagagagaagagaggtaatttgttagcatcaagagtcaagttgctatatggtcaaaggttaaatttatctctaaaaaatggcaggattcaaagttgtacatacatgtgattacttctgttttttacacccacatacagtacaaaagattattaaaaatattcccaaaaggcaggtgcaatgatgcacacttatacccccagccactcaggaggctgatgcaagaggatcgcttgagcccaggagttgaagtccagcctaagcaacatagtgaaaccccatcgccaaaaatataataataattctctcaaaatactaaacagaggtggttttattgataagattttggctgtttggttttccactattctctattggctaaaatttgtttaatgagcatgaaatgtttttattttattttgcttatttttatgattgcaaaaaatgatatgagtttctccctgccaaggcaaaaaaatatatatatacctatattta

SEQ ID NO: 53 - protein, 291 aa, Homo sapiens
  MISC_FEATURE: GULP1 NM_001252668
MNRAFSRKKDKTWMHTPEALSKHFIPYNAKFLGSTEVEQPKGTEVVRDAVRKLKFARHIKKSEGQKIPKVELQISIYGVKILEPKTKEVQHNCQLHRISFCADDKTDKRIFTFICKDSESNKHLCYVFDSEKCAEEITLTIGQAFDLAYRKFLESGGKDVETRKQIAGLQKRIQDLETENMELKNKVQDLENQLRITQVSAPPAGSMTPKSPSTDIFDMIPFSPISHQSSMPTRNGTQPPPVPSRSTEIKRDLFGAEPFDPFNCGAADFPPDIQSKLDEMQRQRWRGSKWD

SEQ ID NO: 54 - DNA, 3523 bp, Homo sapiens
  misc_feature: cDNA GULP1
attccaccctccacccacctccggttccgcgtgcacgcgcgagatagtccagtgggcccacagataacgaccatcagagattaaagaaggaaagtcagcgagcttgaacacaggcgtcccgtgtggaaatgtccaaggagaccgccagaagtgcgcaagccggagtcggctagagtttccttctcaccgagagggggagcccggcgttcccggccgggagcgacccggagtccccagccccgcgtcccagctgccgccagcgccagttttggattcggcggattaggaagaggagggaggggggagagagcgcgaagagggaggggaccgaagctggagggtcccgagtccagcgccgtgttggcgtagagaaactttccctctcggcctcggagacggcgccccggccgtgccggagtggagatcgccaggctcggaggaaccggcagctctccacgcccctgcccgaagcctgacccgactgcctctctcagtgagttatttatgattccatctgatatacataggagagaaactgatagaagaattctgatggcaactgtatgatagaagctatataaagtcaagtgtccattttctttcaactatatttgagcatacccaggatttaagtcgtggaactgaacatttatttggctgatcctcatcatgaaccgtgcttttagcaggaagaaagacaaaacatggatgcatacacctgaagctttatcaaaacatttcattccctataatgcaaagtttcttggcagtacagaagtggaacagccaaaaggaacagaagttgtgagagatgctgtaaggaaactaaagtttgcaagacatatcaagaaatctgaaggccagaaaattcctaaagtggagttgcaaatatcaatttatggagtaaaaattctagaacccaaaacaaaggaagttcaacacaattgccagcttcatagaatatctttttgtgcagatgataaaactgacaagaggatattcactttcatatgcaaagattctgagtcaaataaacatttgtgctatgtatttgacagcgaaaagtgtgctgaagagatcactttaacaattggccaagcatttgacctggcatacaggaaatttctagaatcaggaggaaaagatgttgaaacaagaaaacagatcgcagggttacaaaaaagaatccaagacttagaaacagaaaatatggaacttaaaaataaagtacaagatttggaaaaccaactgagaataactcaagtatcagcacctccagcaggcagtatgacacctaagtcgccctccactgacatctttgatatgattccattttctccaatatcacaccagtcttcgatgcctactcgcaatggcacacagccacctccagtacctagtagatctactgagattaaacgggacctgtttggagcagaaccttttgacccatttaactgtggagcagcagatttccctccagatattcaatcaaaattagatgagatgcagcgccagagatggaggggttcaaaatgggactaactcttgaaggcacagtattttgtctcgacccgttagacagtaggtgctgacatcaagaacaagaaatcctgattcatgttaaatgtgtttgtatacacatgtcatttattattattactttaagataggtattattcatgtgtcaatgtttttgaatattttaatattttgaaaattttctcagttaaatttcctcaccttcactattgatctgtaatttttattttaaaaacagcttactgtaaagtagatcatacttttatgttcctttctgtttctactgtagatgaatttgtaattgaaagacatattatacaaatacctgccttgtgtctgagttctatttagttagcatcttgaaatttgtattcattttccagatggctagtttattaatgatttcccaaaagccataccttaaagataactttttaaattctgaagagacatgccaatgtcaaactaaacatgttctgtttttaaaccaacaaacatgttactattcattggacagatatcattttatgtataaatactgttcacatcactgggaaaatgtaaactttaaacataatgccacaaggtcactaatttctagcaggtaaaattataaggatataaattccaataataaaccaaatgtatttagagtatttattagtaaatgcaaggtgatgttagttatgatcagttatactctaaatatttaatttgttttataaaggtagtgaaaaaatgaaaatttgctatttattaaaaaacattaaatttcattccaaatgagataagtgatattactataacatctaagcatcatctgatttgatattccctaaaaaacatttggaatatatgctatctatagattcagtatctactacccatatttactttaccaaatatatttctcctcactgcataaggactactcttctcatattttcttctttgatgaagatatttttcaccaaagtttattttgtgatgccctcttggttttgatactttaaaatctgtggcacccgttctacatgaattatcaatatttggtaaattcaatctgtatttgttttgttaaagtcaaaaatctcattttccaaaaaaaaaaaaaaaacccagttactgctcagtttagtcttgaacatgagcaataaaattctcttgcatttcattattgatgtgctgatgaacctggacttttaaaaatatttgtttcctatacctttaccctttacctaacagactaatttgtactcagtaaaacaaaaatttatggtcaaaatttctaacttggttcatcacattataagataaataaattaaattaatgaaaatgtgacttagagtaggggtagccctcaaaaatagatttatcatttactcattggaattttcttcaagtgttaaaggtacattttcactaggaaaagaaatcaaatatgcttatgcaatatatatttgtgtgtttttccttaatgttatatggtatatatgagccttcttgtttagtttcttttatctgctaagttgtaccttaattagagggcaatatatgtttcataaagaagagtctttataattttgtttgtcagatagtattttggaatttgtataataaggatgtttagaagccatataagtggctttttttaacagatagaatttgtatttttattgtactttaaaaagatttatgtaataggtatatatttagtggccatttattatcaatggtaacacaatggagtactaagatggtatttgcacatttaagatatgttactttaccaatttttaatggtaatcaactctgctactggcatgatgaaatagtacataactggtcattaattatgaacatttatttctccagtgcgtttttatgaagatctggttgaaaattgtatttctatgtaaactcaacgatatgtttggttttcctgaaaataaatgattttaaataaaaaaaaaaaaaaaa

SEQ ID NO: 55 - protein, 375 aa, Homo sapiens
  MISC_FEATURE: NDRG3 NM_032013
MDELQDVQLTEIKPLLNDKNGTRNFQDFDCQEHDIETTHGVVHVTIRGLPKGNRPVILTYHDIGLNHKSCFNAFFNFEDMQEITQHFAVCHVDAPGQQEGAPSFPTGYQYPTMDELAEMLPPVLTHLSLKSIIGIGVGAGAYILSRFALNHPELVEGLVLINVDPCAKGWIDWAASKLSGLTTNVVDIILAHHFGQEELQANLDLIQTYRMHIAQDINQDNLQLFLNSYNGRRDLEIERPILGQNDNKSKTLKCSTLLVVGDNSPAVEAVVECNSRLNPINTTLLKMADCGGLPQVVQPGKLTEAFKYFLQGMGYIPSASMTRLARSRTHSTSSSLGSGESPFSRSVTSNQSDGTQESCESPDVLDRHQTMEVSC

SEQ ID NO: 56 - DNA, 3031 bp, Homo sapiens
  misc_feature: cDNA NDRG3
acctcccccgcccctctcgccgccgccgccgccgccgccgccgccgccgccgccgccgctgctgctgcactgacggcgggtgcccgcgcctcagagttactgatttattcttgagattcctctactctcgttatctgacctcatggatgaacttcaggatgttcagctcacagagatcaaaccacttctaaatgataagaatggtacaagaaacttccaggactttgactgtcaggaacatgatatagaaacaactcatggtgtggtccacgtcactataagaggcttacccaaaggaaacagaccagttatactaacatatcatgacattggcctcaaccataaatcctgtttcaatgcattctttaactttgaggatatgcaagagatcacccagcactttgctgtctgtcatgtggatgccccaggccagcaggaaggtgcaccctctttcccaacagggtatcagtaccccacaatggatgagctggctgaaatgctgcctcctgttcttacccacctaagcctgaaaagcatcattggaattggagttggagctggagcttacatcctcagcagatttgcactcaaccatccagagcttgtggaaggccttgtgctcattaatgttgacccttgcgctaaaggctggattgactgggcagcttccaaactctctggcctgacaaccaatgttgtggacattattttggctcatcactttgggcaggaagagttacaggccaacctggacctgatccaaacctacagaatgcatattgcccaagacatcaaccaagacaacctgcagctcttcttgaattcctacaatggacgcagagacctggagatcgaaagacccatactgggccaaaatgataacaaatcaaaaacattaaagtgttctactttactggtggtaggggacaattcgcctgcagttgaggctgtggtcgaatgcaattcccgcctgaaccctataaatacaactttgctaaagatggcggactgtgggggactgccccaggtagttcagcctgggaagctcaccgaggccttcaagtactttttgcagggaatgggctacataccatctgccagcatgactcggctcgcccgatcacgaacccactcaacctcgagtagcctcggctctggagaaagtcccttcagccggtctgtcaccagcaatcagtcagatggaactcaagaatcctgtgagtcccctgatgtcctggacagacaccagaccatggaggtgtcctgctaagcagatgctcctcccctggaccattgcaagtccatccttcaaatgaccactccataatataacatttcatccagtaaactggcctctactatctttaactcatgcatggccactgaacctctctctagtagcctggatttatcattctctctgcctgcccacccccttttttgtatagcccaagaaccacttccatgccatactgtaacattccaacatctttagctgatcagatctctccatatcctctcttgccagctttttcccgtgctcccccaactatgtatcagataagattctttgatcccgactctgtgtgtgcgagcacgcgtgtctgtgtttgtgtgtgcatagttctgtggttttagacacgctttcttgtagtgcttctgcaaaaaacaaaaaagggacttattttgcattctcaatggtgtttttaagggaattaggcagaacagatttctaggttgggtaggccactgcattctcttttgtttgcaaattggtcaacaaaatttgcaaagtgatttcaggagagagcagctttgaggaatgtggaaaatcataattgccgtctggaccattgattgattgtgaccagtagcagaagggtgcctgttacatagagaggctccttctgtccaaatgaatttctgtatactcttctataaataaaagggaggaatatattctgctggaagcccatgaaccatcgctgaggttctgatacaacatagagttttttccaaggagtgaatgtggtttaattactggactctcttagcacaggaaggtggaaacaaaatgccaggcctctgctctgaagagcaaaactgctgtcgctgcagtatctgataccagacatccacatatccacaagaagtgcctcttaggtctgtgacagagagtgtgtctccattcctcagttcccagaaaggggagaggtttggcctaaaaagcatgtagatgggggagaaatgggtggggggagaggaacagccattaacacagtatcatgtttaacaagtatagccttgatttcagtagatgtaatggaagccaaattaaattgatacagaacccatttctcagagtctttttttttttttgagacagagtctcgctgttacccaggctggagtgcaatggcgcaaacttggctcactgcaacctctgccccctgggttcaagcgattctcctgcctcagcctcccgagtagctgagactacaggcacctgccaccatacccagctaagttatgtatttttagtagagatggagtttcaccatgttggccaggctggtctcaaactcctgacctcaggtgatccacctgcctcagcctcccaaagtgctgggattacaggcatgagtcattgctcccagccattagaaagattgttaatcctatgaactcccttttgtaggagagaaagggccaatctgtaggggtagccctgtccaggtaaagttgttttcagcctcatgtctactgttaggtgagggagtcacagccagacagagagtattgctggagggtgagagaattgtggagaccaactaccacatagcaagagcccagctcttgggagcattgagatgtaagctcagggttacacagttccaaatcttgggaaggggcttttcagacagactgtttgctttctgctgagataaggaatgcatcactctgccagagtatgactttttacagattattaaataaagctgcatatgtctcattgttacctgaaaaaaaa

SEQ ID NO: 57 - DNA, 20 bp, Artificial Sequence
  PRIMER SENSE OVGP1
tatgtcccgtatgccaacaa

SEQ ID NO: 58 - DNA, 20 bp, Artificial Sequence
  PRIMER ANTISENSE OVGP1
tccatgtccaatgtccacac

SEQ ID NO: 59 - DNA, 19 bp, Artificial Sequence
  PRIMER SENSE S100A14
agcggctgccaacagatca

SEQ ID NO: 60 - DNA, 21 bp, Artificial Sequence
  PRIMER ANTISENSE S100A14
actgtgtctggtcctttggtg

SEQ ID NO: 61 - DNA, 23 bp, Artificial Sequence
  PRIMER SENSE SERPINB5
catgttcatcctactacccaagg

SEQ ID NO: 62 - DNA, 26 bp, Artificial Sequence
  PRIMER ANTISENSE SERPINB5
tctgagttgagttgtttttcaatctt

SEQ ID NO: 63 - DNA, 20 bp, Artificial Sequence
  PRIMER SENSE SPRR3b
accagagccatgtccttcaa

SEQ ID NO: 64 - DNA, 20 bp, Artificial Sequence
  PRIMER ANTISENSE SPRR3b
atctggtggttggcttctca

SEQ ID NO: 65 - DNA, 20 bp, Artificial Sequence
  PRIMER SENSE ENPP3
tgtcacgggcttgtatccag

SEQ ID NO: 66 - DNA, 20 bp, Artificial Sequence
  PRIMER ANTISENSE ENPP3
tgccaccaggctggattatt

SEQ ID NO: 67 - DNA, 19 bp, Artificial Sequence
  PRIMER SENSE CLUAP1
ccaagccacagacagccat

SEQ ID NO: 68 - DNA, 19 bp, Artificial Sequence
  PRIMER ANTISENSE CLUAP1
tctccaccttgcatcgtgc

SEQ ID NO: 69 - DNA, 20 bp, Artificial Sequence
  PRIMER SENSE CLCA4
tcacttcacccctgaccttc

SEQ ID NO: 70 - DNA, 20 bp, Artificial Sequence
  PRIMER ANTISENSE CLCA4
gagcccactcatggacaaac

SEQ ID NO: 71 - DNA, 24 bp, Artificial Sequence
  PRIMER SENSE CEACAM5
caataggaccacagtcacgacgat

SEQ ID NO: 72 - DNA, 22 bp, Artificial Sequence
  PRIMER ANTISENSE CEACAM5
ggttggagttgttgctggtgat

SEQ ID NO: 73 - DNA, 20 bp, Artificial Sequence
  PRIMER SENSE RNASE3
cagagactgggaaacatggt

SEQ ID NO: 74 - DNA, 20 bp, Artificial Sequence
  PRIMER ANTISENSE RNASE3
aaccactgagccctcgtaaa